SEQUENCE LISTING (17 sequences)

SEQ ID NO: 1 - protein, 299 aa, Streptomyces sp. MA37
MAANGSQRPIIAFMSDLGTTDDSVAQCKGLMHSICPGVTVVDVCHSMTPWDVEEGARYIVDLPRFFPEGTVFATTTYPATGTTTRSVAVRIRQAAKGGARGQWAGSGDGFERADGSYIYIAPNNGLLTTVLEEHGYIEAYEVTSTKVIPANPEPTFYSREMVAIPSAHLAAGFPLAEVGRRLDDSEIVRFHRPAVEISGEALSGVVTAIDHPFGNIWTNIHRTDLEKAGIGQGKHLKIILDDVLPFEAPLTPTFADAGAIGNIAFYLNSRGYLSLARNAASLAYPYNLKAGLKVRVEAR

SEQ ID NO: 2 - DNA, 902 bp, Streptomyces sp. MA37
tggtggctgcaaacggctctcagcgccccatcatcgccttcatgagcgacctcggcaccacggacgactcggtggcccagtgcaagggcctcatgcacagcatctgccccggcgtcacggtggtggacgtctgccactccatgacgccgtgggacgtcgaggagggtgcgcgctacatcgtcgacctcccccgcttcttccccgagggcacggtgttcgccaccaccacctatcccgcgaccggcaccacgacccgctccgtggccgtgcggatccggcaggcggccaagggcggcgcccgcggccagtgggccggctccggcgacggcttcgagcgggccgacggctcgtacatctacatcgcgcccaacaacggcctgctgaccaccgtgctcgaggagcacggatacatcgaggcctacgaggtcacgtccaccaaggtcatccccgcgaaccccgaaccgaccttctacagccgcgagatggtggccatcccctccgcccacctcgccgccggcttcccgctggccgaggtcggccgccggctcgacgacagcgagatcgtccgcttccaccggcccgcggtcgagatctccggggaggcgctctccggcgtcgtcaccgcgatcgaccaccccttcggcaacatctggaccaacatccaccgcacggacctggagaaggccggcatcggccaggggaagcacctgaagatcatcctcgacgacgtcctgccgttcgaggccccgctcaccccgaccttcgcggacgccggggcaatcggcaacatcgccttctacctcaacagccgcggctacctgtccctcgcccgcaacgccgccagcctcgcctacccctacaacctcaaggccggtctgaaggtccgcgtggaagcgcgctga

SEQ ID NO: 3 - DNA, 900 bp, Streptomyces sp. MA37
atggccgccaacggcagccagcgcccgatcatcgcgttcatgtccgacctgggcaccaccgacgacagcgtggcgcagtgcaagggcctgatgcacagcatctgccccggtgtgaccgtggtggatgtgtgccacagcatgaccccgtgggacgtcgaggagggcgcccgttacatcgtggacctgccgcgtttcttcccggagggcaccgtcttcgccaccaccacctatcccgccaccggcaccaccacccgcagcgtggccgtgcgtatccgccaggccgccaaaggcggcgcccgtggccagtgggccggcagcggcgacggcttcgaacgtgccgacggcagctacatctacatcgccccgaacaacggcctgctgaccaccgtgctggaggaacacggctatatcgaggcctacgaggtgaccagcaccaaggtgatcccggcgaacccggagccgaccttctacagccgcgaaatggtggccatcccgtccgcccacctggccgccggcttcccgctggccgaggtgggccgtcgtctggatgacagcgagatcgtccgtttccaccgccccgccgtcgagatctccggcgaagccctgagcggcgtggtgaccgccatcgaccacccgttcggcaacatctggaccaacatccaccgtaccgacctggaaaaggccggcatcggccagggcaaacacctgaagatcatcctggacgacgtgctgccgttcgaagccccgctgacccccaccttcgccgacgccggcgccatcggcaacatcgccttctacctgaacagccgcggctatctgagcctggcccgcaacgccgccagcctggcctatccgtacaacctgaaagccggcctgaaggtgcgcgtggaggcccgttga

SEQ ID NO: 4 - DNA, 248 bp, Pseudomonas syringae
tttggccctctttcgtaagcggtgactcgaagacgccttcgcttgcctcgtactccgaaacacgattttacattttggacagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaa

SEQ ID NO: 5 - DNA, 276 bp, Pseudomonas syringae
atcctcttttggccctctttcgtaagcggtgactcgaagacgccttcgcttgcctcgtactccgaaacacgattttacattttggacagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaaatgtctaaatttcgacgacct

SEQ ID NO: 6 - DNA, 255 bp, Pseudomonas syringae
atcctcttttggccctctttcgtaagcggtgactcgaagacgccttcgcttgcctcgtactccgaaacacgattttacattttggacagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaa

SEQ ID NO: 7 - DNA, 264 bp, Artificial sequence
  Synthetic construct
  misc_feature (1)..(264): PEM7 promoter, Pseudomonas syringae FRS and first codons of EriCF
atcctctttgttgacaattaatcatcggcatagtatatcggcatagtataatacgacaaggtgaggaactaaaccagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaaatgtctaaatttcgacgacct

SEQ ID NO: 8 - DNA, 243 bp, Artificial sequence
  Synthetic construct
  misc_feature (1)..(243): PEM7 promoter, Pseudomonas syringae FRS
atcctctttgttgacaattaatcatcggcatagtatatcggcatagtataatacgacaaggtgaggaactaaaccagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaa

SEQ ID NO: 9 - DNA, 375 bp, Pseudomonas putida
atgattgcactcatcgccgcggtcagcgccggtggtattgctggtaccttgttgcgctttgctaccaccaactgggtggcggctcactggccacggcacttctatgccggtacgctggcggtcaacctggttggctgcctgctgatcggcttgctctatggcttgttcttgcacaagccgctggcgccggtcgaattgcgcgccggcctgattgtcggctttctcggtggcctgacgaccttttcctccttctcgctcgataccgtgcgcctgatggaaagcgggcaagtgccccttgccttgggctataccagtatcagcgtggtgggcgggctgctcgcaacctgggctggcctgtccctgacccgattctga

SEQ ID NO: 10 - protein, 124 aa, Pseudomonas putida
MIALIAAVSAGGIAGTLLRFATTNWVAAHWPRHFYAGTLAVNLVGCLLIGLLYGLFLHKPLAPVELRAGLIVGFLGGLTTFSSFSLDTVRLMESGQVPLALGYTSISVVGGLLATWAGLSLTRF

SEQ ID NO: 11 - protein, 298 aa, Streptomyces sp. MA37
MRESGGAAFYSADLGIIGGSGLYAFDGLQDVTEAELRTPYGDPSGPLVLGTAGNRRIAFLPRHGRGHRIAPSQVPARANVYALKSLGVREVISVSAVGSLRGDYAPGDMVVLDQLFDRTAGLRPASFFGDGMVAHVSMADPYCGRLRPFLEQVAAEEHPTVHGSGTYCCIEGPQFSTRAESEVYRSWGMDVIGMTALPEAKLAREAELCYTGLALVTDYDCWHPGHASVDAQGVAEIMEANVAAARRVVMRFAESSKPAPCVCQRALDKAIMTDSQIIQGGPGNTVPTLLRDRIAPVV

SEQ ID NO: 12 - DNA, 897 bp, Streptomyces sp. MA37
atgcgagaatcgggcggcgcggccttctactcggccgacctcggaatcatcggcggcagcgggctctacgccttcgacggcctgcaggacgtgacggaggccgaactgcggaccccgtacggcgaccccagcgggccgctggtcctgggcacggccgggaaccgccgcatcgccttcctgccgcgccacgggcgcggccaccgcatagccccgtcgcaggtcccggcgcgggccaacgtctacgcgctgaagtccctcggcgtccgcgaagtgatcagcgtcagcgccgtgggcagcctgcgcggcgactatgccccgggcgacatggtcgtcctcgaccagctcttcgaccgcaccgccgggctgcggcccgcctcgttcttcggggacgggatggtggcccacgtgtcgatggcggacccgtactgcggccggctgcgccccttcctcgaacaggtggcggccgaggagcatcccacggtccacgggtccgggacgtactgctgcatcgaggggccccagttctccacccgcgccgagtccgaggtgtaccgctcatgggggatggacgtcatcggcatgacggcgctgccggaggcgaaactcgcccgtgaggctgaactctgctacaccgggctcgccctcgtgacggactacgactgctggcacccgggccacgcgtcggtggacgcccagggggtcgcggagatcatggaggccaacgtggccgccgcccggagggtcgtcatgcggttcgccgagtcgtcgaagcccgcgccctgcgtgtgtcagcgggcgctggacaaagccattatgacggacagtcagatcattcagggaggtccggggaacacggtgcccacgctcctgcgcgaccggatcgcgccggtggtgtga

SEQ ID NO: 13 - DNA, 897 bp, Streptomyces sp. MA37
atgcgcgagagcggcggcgccgccttctacagcgccgatctgggcatcatcggcggcagcggcctgtacgccttcgacggcctgcaagacgtgaccgaagccgaactgcgcaccccgtacggcgacccgagcggcccgctggtgctgggcaccgccggcaaccgtcgtatcgcgttcctgccgcgccatggccgcggccaccgtatcgccccgagccaggtgccggcccgtgccaacgtgtatgccctgaagagcctgggcgtgcgtgaggtgatcagcgtgtccgccgtgggctccctgcgcggcgactatgcccccggtgacatggtggtgctggatcagctgttcgaccgcaccgcgggcctgcgtccggccagcttcttcggcgatggcatggtggcccatgtgtcgatggccgacccgtattgcggccgtctgcgcccgttcctggaacaggtcgccgccgaggaacacccgaccgtgcatggcagcggcacctactgctgcatcgagggcccgcagttcagcacccgcgccgaaagcgaggtgtaccgcagctggggcatggacgtcatcggcatgaccgccctgcccgaggccaaactggcgcgtgaagccgagctgtgctataccggcctggccctggtgaccgattacgactgctggcaccccggccacgccagcgtggatgcccagggcgtggccgaaatcatggaggcgaacgtggccgccgcccgccgtgtggtgatgcgtttcgcggaaagcagcaagcccgccccgtgcgtctgccagcgtgccctggataaggccatcatgaccgatagccagatcatccagggcggccccggtaacaccgtgccgaccctgctgcgcgatcgcatcgccccggtcgtgtga

SEQ ID NO: 14 - DNA, 738 bp, Escherichia coli
atggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactgcgcgacgtcgttatcggtatgggtgcctgcaccgattccaaagttaaccgcatccgttttaaagaccatgactttgccgctatcgctgacttcgacatggtgcgtaacgcagtagatgcagctaaagcactgggtattgatgctcgcgtgggtaacctgttctccgctgacctgttctactctccggacggcgaaatgttcgacgtgatggaaaaatacggcattctcggcgtggaaatggaagcggctggtatctacggcgtcgctgcagaatttggcgcgaaagccctgaccatctgcaccgtatctgaccacatccgcactcacgagcagaccactgccgctgagcgtcagactaccttcaacgacatgatcaaaatcgcactggaatccgttctgctgggcgataaagagcatcatcaccaccaccattga

SEQ ID NO: 15 - protein, 239 aa, Escherichia coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 16 - DNA, 717 bp, Escherichia coli
atgaaaatcggcatcattggtgcaatggaagaagaagttacgctgctgcgtgacaaaatcgaaaaccgtcaaactatcagtctcggcggttgcgaaatctataccggccaactgaatggaaccgaggttgcgcttctgaaatcgggcatcggtaaagtcgctgcggcgctgggtgccactttgctgttggaacactgcaagccagatgtgattattaacaccggttctgccggtggcctggcaccaacgttgaaagtgggcgatatcgttgtctcggacgaagcacgttatcacgacgcggatgtcacggcatttggttatgaatacggtcagttaccaggctgtccggcaggctttaaagctgacgataaactgatcgctgccgctgaggcctgcattgccgaactgaatcttaacgctgtacgtggcctgattgttagcggcgacgctttcatcaacggttctgttggtctggcgaaaatccgccacaacttcccacaggccattgctgtagagatggaagcgacggcaatcgcccatgtctgccacaatttcaacgtcccgtttgttgtcgtacgcgccatctccgacgtggccgatcaacagtctcatcttagcttcgatgagttcctggctgttgccgctaaacagtccagcctgatggttgagtcactggtgcagaaacttgcacatggccatcatcaccaccaccattga

SEQ ID NO: 17 - protein, 232 aa, Escherichia coli
MKIGIIGAMEEEVTLLRDKIENRQTISLGGCEIYTGQLNGTEVALLKSGIGKVAAALGATLLLEHCKPDVIINTGSAGGLAPTLKVGDIVVSDEARYHDADVTAFGYEYGQLPGCPAGFKADDKLIAAAEACIAELNLNAVRGLIVSGDAFINGSVGLAKIRHNFPQAIAVEMEATAIAHVCHNFNVPFVVVRAISDVADQQSHLSFDEFLAVAAKQSSLMVESLVQKLAHG

(19) United States
(12) Patent Application Publication
(10) Pub. No.: US 2024/0263207 A1
(43) Pub. Date: Aug. 8, 2024

(54) IN VIVO FLUORINATION

(71) Applicant: Danmarks Tekniske Universitet, Kgs. Lyngby (DK)

(72) Inventors: Pablo Iván Nikel, Copenhagen (DK); Patricia Maria Calero Valdayo, Gentofte (DK)

(73) Assignee: Danmarks Tekniske Universitet, Kgs. Lyngby (DK)

(21) Appl. No.: 17/286,855

(22) PCT Filed: Oct. 23, 2019
(86) PCT No.: PCT/EP2019/078790

§ 371 Date: Apr. 20, 2021

(30) Foreign Application Priority Data
Oct. 23, 2018 (EP) 18201983.6

(51) Int. Cl. C12P 19/40; C12N 1/20; C12N 9/10; C12N 9/24; C12N 15/78; C12R 1/40

(52) U.S. Cl. CPC C12P 19/40; C12N 1/205; C12N 9/1077; C12N 9/1085; C12N 9/2497; C12N 15/78; C12R 2001/40; C12Y 204/02028; C12Y 205/01063; C12Y 302/02009

(57) ABSTRACT

The present invention relates to a cell capable of producing a fluorinated, a chlorinated or a brominated compound, methods for producing fluorinated, chlorinated or brominated compounds in a cell and expression systems therefor.

### TECHNICAL FIELD

The present invention relates to a cell capable of producing a fluorinated, a chlorinated or a brominated compound, methods for producing fluorinated, chlorinated or brominated compounds in a cell and expression systems therefor.

### SEQUENCE LISTING

The present application contains a Sequence Listing which has been submitted in ASCII format via EFS-Web and is hereby incorporated by reference in its entirety. Said ASCII copy, created on May 17, 2023, is named “16DMTU-HO78402PA_SEQ.txt” and is 24,576 bytes in size.

### BACKGROUND

Halogenated molecules represent a third of the drugs currently on clinical trials. Specifically, almost 25% of pharma molecules contain fluorine (F) and latest figures indicate that the market of fluorinated compounds for the pharma industry will continue expanding in the upcoming years (Zhou 2016). F-containing organic compounds are extremely relevant from a pharmaceutical and industrial point of view, since F enhances lipophilicity, helping cell membrane penetration and increasing drug bioavailability (Zhang 2012). Although most of the pharmaceutically-relevant drugs that we rely on are provided, directly or via a precursor molecule, by nature, a missing key structure in this picture is F-containing organic molecules. Only 12 naturally-occurring organofluorines have been identified (e.g., fluoroacetate, found in tropical plants). This is due to the huge thermodynamic constraints that biochemical reactions involving F need to overcome, and the toxicity of low-molecular-weight fluorinated compounds (O'Hagan 2008).

There is just one microbial enzymatic reaction that occurs in the high-G+C content bacterium Streptomyces (Deng 2004). The fluorination pathway of Streptomyces cattleya uses S-adenosyl-L-methionine (SAM) and inorganic fluoride (F⁻) as substrates, generating 5′-fluoro-5′-deoxyadenosine (5′-FDA) and L-methionine in the first step catalysed by the fluorinase, which is encoded by flA. Although the fluorinase of S. cattleya was the first enzyme to form a C—F bond to be identified and isolated (O'Hagan 2002), other fluorinases have been identified and described in other actinomycetes. For example, fluorinases from Streptomyces sp. MA37, Nocardia brasilensis, and Actinoplanes sp. were identified by genome mining by comparison to the fluorinase from S. cattleya (Deng 2014). A fluorinase was also identified in Streptomyces xinghaiensis (Ma et al., 2016). Whereas this enzyme has been thoroughly characterized in vitro (O'Hagan 2002, Zhu 2007), its wide biotechnological exploitation in vivo has been limited mostly because of the high toxicity of the resulting fluorinated molecules and the complexity of the C—F bond chemistry. The only successful in vivo fluorometabolite production was performed in Salinospora tropica, where the chlorinase from this organism was replaced with FlA to produce fluorosalinosporamide (Eustaquio 2010). After that, further engineering of the fluorinases to improve their efficiency, activity and increase of their substrate range has been done (Sun 2016, Yeo 2017).

Moreover, successful protein expression for production of chemicals requires significant efforts to optimize the chassis and tools to achieve sufficiently high titers and yields. One of the challenges as well is the reduction of costs derived, for example, of the additives that need to be used in the process, such as inducers or antibiotics. To avoid the use of inducers, some strategies have been proposed, such as the use of thermosensitive or quorum sensing based expression systems. Another alternative is the use of riboswitches that recognize a specific molecule that is involved in the production process and triggers the expression of the pathway when it is present. Riboswitches are regulatory elements that are found in the 5′-UTR of bacterial mRNA and that post-transcriptionally control the gene expression upon binding to a specific metabolite. They have been used widely as biosensors, which respond with a detectable output to the presence of a certain molecule, providing easy screening methods of high-producing strains (Liu 2015). However, the use of riboswitches has been limited by their contextual effect on the expression gene (Kent 2018), although they have been used successfully in inducible expression systems (Morra 2015). A fluoride responsive riboswitch (FRS) have been described in numerous bacterial and archaeal organisms, controlling the expression of genes involved in the detoxification of the ion, typically transporters (Stockbridge 2012, Clayton Speed 2018). The sequence, structure, and mechanism of action have been studied for the riboswitch from Pseudomonas syringae and Bacillus subtilis, which have been used as well as biosensors to detect the intracellular concentration of NaF in Escherichia coli (Baker 2012).

Finally, the election of a suitable host for production presents a challenge that has considerable impact in the field of metabolic engineering. Environmental bacteria, that naturally thrive under rough physicochemical conditions, are evolutionarily pre-endowed with many physiological traits (e.g., stress endurance and metabolic plasticity) desirable in industrial settings (Danchin 2012, Calero and Nikel 2018).

### SUMMARY

In a first aspect, is provided a cell capable of producing a fluorinated, a chlorinated or a brominated compound from a substrate in the presence of fluoride, chloride or bromide, respectively, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
wherein the cell is capable of expressing the activator of transcription and the fluorinase at least in the presence of said inducer.

Also provided is a cell capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch,
   wherein transcription of the fluorinase gene from the first promoter is induced in the presence of an inducer, wherein said riboswitch is responsive to said inducer;
wherein the cell is capable of expressing the fluorinase at least in the presence of said inducer.

Also provided is an expression system for expression in a cell, comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter.

Also provided is an expression system for expression in a cell, comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch,
   wherein transcription of the fluorinase gene from the first promoter is induced in the presence of an inducer, wherein said riboswitch is responsive to said inducer,
   whereby the cell is capable of producing a fluorinated product such as 5′-fluoro-5′-deoxyadenosine or 5′-deoxy-5′-fluoro-D-ribose 1-phosphate.

Also provided is a method for in vivo fluorination, chlorination or bromination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
   b) a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate selected from a fluoride salt, a chloride salt or a bromide salt,
   whereby transcription of the gene encoding an activator of transcription is induced,
   thereby inducing transcription of the fluorinase gene,
thereby inducing fluorination, chlorination or bromination of the substrate to yield a fluorinated, a chlorinated or a brominated product.

Also provided is a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) a second nucleic acid comprising a riboswitch,
   wherein transcription of the fluorinase gene from the first promoter is induced in the presence of an inducer, wherein said riboswitch is responsive to said inducer.

Also provided is a fluorinated, a chlorinated or a brominated product obtainable by the methods described herein.

Also provided is a fluorinated compound obtainable by the methods described herein.

Also provided is a composition comprising a fluorinated, a chlorinated or a brominated product obtainable by the methods described herein.

Also provided is a composition comprising a fluorinated product obtainable by the methods described herein.

Also provided is a method for manufacturing a fluorinated, a chlorinated or a brominated compound of interest, said method comprising the steps of:
i) providing a fluorinated, a chlorinated or a brominated product by the methods described herein; and
ii) optionally converting said fluorinated, chlorinated or brominated product to the fluorinated, chlorinated or brominated compound of interest.

Also provided is a method for manufacturing a fluorinated compound of interest, said method comprising the steps of:
i) providing a fluorinated, a chlorinated or a brominated product by the methods described herein; and
ii) optionally converting said fluorinated product to the fluorinated compound of interest.

Also provided herein is the fluorinated, chlorinated or brominated product or the composition described herein for use as a medicament.

Also provided herein is the fluorinated product or the composition described herein for use as a medicament.

### DESCRIPTION OF THE DRAWINGS

FIG. 1: Scheme of the FRS-T7 system. The T7 RNA polymerase (dark grey) is integrated in the genome, inducing the expression of the plasmid pFB-PT7flA1, which contains the fluorinase gene flA1 (light grey) under the regulation of promoter PT7, in the presence of NaF (diamonds). FlA1 (light grey circles) catalyses the biofluorination reaction converting SAM (pentagon) and NaF into 5′-FDA.

FIG. 2: Different constructs containing the F⁻ responsive riboswitch (FRS) with different combinations of the original promoter of the gene eriC^{F} (PEriCF in arrows), a constitutive promoter (PEM7 in arrows), and the sequence encoding the first 8 amino acids of the gene eriC^{F} (grey box). GFP is indicated as white boxes and terminators as light grey boxes.

FIG. 3: Normalized fluorescence of the four different FRS constructs of FIG. 2 and the empty vector in response to different concentrations of NaF in P. putida KT2440. Errors bars correspond to standard deviations of 3 different biological replicates.

FIG. 4: Construction of the FRS-T7RNA polymerase system. The FRSv1 construct regulates the expression of the T7 RNA polymerase (in dark grey). All this is in this embodiment contained in the mini-Tn7 (left and right sites of the mini-Tn7 are depicted in grey boxes), along with a gentamycin resistance cassette (GmR). This construct is integrated in the genome of the production platform strain. A plasmid containing the GFP with a PT7 promoter is also transformed in the production platform strain.

FIG. 5: Normalized fluorescence (bars) and growth (A₆₀₀, dots) of P. putida KT2440::FRS-T7RNAP induced with different concentrations of NaF. Error bars correspond to standard deviations of three biological replicates.

FIG. 6: Minimal inhibitory concentration (MICs) assays of P. putida KT2440 in M9 minimal medium with different concentrations of NaF. In the graph, the A₆₀₀ is shown after 20 hours of incubation. The points of three different biological replicates are represented as dots.

FIG. 7: Minimal inhibitory concentration (MICs) assays of P. putida KT2440ΔcrcB in M9 minimal medium with different concentrations of NaF. In the graph, the A₆₀₀ is shown after 20 hours of incubation. The points of three different biological replicates are represented as dots.

FIG. 8: Aliquots of P. putida KT2440 and P. putida KT2440ΔcrcB cultures at different dilutions spotted onto LB plates containing 0, 0.1, and 10 mM of NaF.

FIG. 9: Normalized fluorescence (bars) and growth (A₆₀₀, dots) of P. putida KT2440ΔcrcB::FRS-T7RNAP induced with different concentrations of NaF as indicated. Error bars correspond to standard deviations of three biological replicates.

FIG. 10: 10A: Comparison of the areas of 5′-FDA found in the cell extracts in vivo in induced cultures of P. putida KT2440::FRS-T7RNAP harboring the plasmid pFB-PT7flA1, normalized against the 5′-FDA standard areas. The error bars correspond to the standard deviations of two different biological replicates. 10B: Comparison of the concentrations of 5′-FDA content per mg of cell dry weight (CDW) found metabolite extractions of in vivo induced cultures of P. putida KT2440::FRS-T7RNAP harboring the plasmid pFB-PT7flA1. The error bars correspond to the standard deviations of two different biological replicates.

FIG. 11: Final growth of induced cultures of P. putida KT2440::FRS-T7RNAP harboring the plasmid pFB-PT7flA1 (dots) or empty plasmid pSEVA231 (squares). The points of two different biological replicates are represented.

FIG. 12: Construction of plasmid pFB-FRSflA1, containing the first version of the fluoride responsive riboswitch (FRSv1) controlling the expression of the gene flA1 (empty box).

FIG. 13: In vivo production of 5′-FDA in P. putida KT2440 strain with plasmid pFB-FRSflA1, after 20 and 48 hours of induction with 15 mM of NaF. The error bars correspond to the standard deviations of three different biological replicates.

FIG. 14: Scheme of the FRS-T7 system controlling the expression of the gene flB1 coding a purine nucleotide phosphorylase (PNP). The T7 RNA polymerase (dark grey) is integrated in the genome, inducing the expression of the plasmid pFB-PT7flB1(NHis), which contains the PNP gene flB1 (light grey) with a 6× histidine tag in the amino terminal of the protein (dark grey square). The expression is controlled by the regulation of promoter PT7, in the presence of NaF (diamonds). FlB1 (light grey circles) catalyzes the phosphorylation reaction converting 5′-FDA into 5′-FDRP.

FIG. 15: In vitro degradation of 5′-FDA by cell extracts of P. putida KT2440::FRS-T7RNAP expressing the PNP FlB1 with a 6× histidine tag in the amino terminal of the protein (pFB-PT7flB1-NHis), and a negative control (empty vector pSEVA231). The black bars represent the initial concentration of 5′-FDA added to the reaction containing the cell extracts, and in grey bars the concentration of 5′-FDA after 20 hours of incubation is shown.

FIG. 16: In vitro conversion of 5′-FDA (light grey) into 5′-FDRP (dark grey) by the PNP DeoD, and into 5′-FDR (black) by the Pfs nucleosidase. The conversion of the empty plasmid pSEVA231-T7Pr is also shown. The error bars correspond to the standard deviations of two different biological replicates.

FIG. 17: Scheme of the FRS-T7 system controlling the expression of the genes flA1 and flB1. The T7 RNA polymerase (dark grey) is integrated in the genome, inducing the expression of the plasmid pFB-PT7flA1B1, which contains the fluorinase gene fla1 as well as the PNP gene flB1 (light grey) with a 6× histidine tag in the amino terminal of the protein (dark grey square). The expression is controlled by the regulation of promoter PT7, in the presence of NaF (diamonds). FlA1 together with FlB1 (light grey circles) catalyze the reaction converting F⁻ into 5′-FDRP.

FIG. 18: LC-MS chromatogram showing the peak area corresponding to 5′-FDRP found in the supernatants of induced cultures of P. putida KT2440::FRS-T7RNAP containing NaF and the plasmid pFB-PT7flA1B1 (dark grey line). No peak could be detected in the same conditions with cells with the empty plasmid (light grey). The result is from a representative sample of two replicates.

### DETAILED DESCRIPTION OF THE INVENTION

The present disclosure relates to cells capable of producing a fluorinated, a chlorinated or a brominated compound, methods for producing fluorinated, chlorinated or brominated compounds in a cell and expression systems therefor.

### Definitions

### Fluorinase

The fluorinase enzyme (EC 2.5.1.63, also known as adenosyl-fluoride synthase) catalyses the reaction between fluoride ion and the co-factor S-adenosyl-L-methionine (SAM) to generate L-methionine and 5′-fluoro-5′-deoxyadenosine (5′-FDA), the first committed product of the fluorometabolite biosynthesis pathway. Fluorinase was originally isolated from the soil bacterium Streptomyces cattleya, and is the only known enzyme capable of catalysing the formation of a carbon-fluorine bond, the strongest single bond in organic chemistry.

Fluorinase catalyses the reaction:

S-adenosyl-L-methionine+fluoride<=>5′-fluoro-5′-deoxyadenosine+L-methionine.

It can however also act on other substrates besides SAM, for example methylaza-SAM derivatives. Fluorinase can also catalyse the following reactions:
S-adenosyl-L-methionine+chloride<=>5′-deoxy-5′-chloroadenosine+L-methionine;
5′-deoxy-5′-fluoroadenosine+L-selenomethionine<=>Se-adenosyl-L-selenomethionine+fluoride; 2′-deoxyadenosyl-L-methionine+fluoride<=>2′-deoxy-5′-fluoroadenosine+L-methionine;
S-adenosyl-L-methionine+bromide<=>5′-deoxy-5′-bromoadenosine+L-methionine.

Fluorinase can thus catalyse fluorination reactions (addition of an F atom to a compound using fluoride as co-substrate), chlorination reactions (addition of a CI atom on a compound using chloride as co-substrate) and bromination reactions (addition of a Br atom on a compound using bromide as co-substrate).

### Phosphorylase

The term refers herein to S-methyl-5′-thioadenosine phosphorylase (EC 2.4.2.28), which is capable of catalysing the following reaction:
S-methyl-5′-thioadenosine+phosphate<=>adenine+S-methyl-5-thio-alpha-D-ribose 1-phosphate

The enzyme is also capable of catalysing phosphorylation of fluorinated adenosine.

### Cell

In a first aspect, is provided a cell capable of producing a fluorinated, a chlorinated or a brominated compound from a substrate in the presence of fluoride, chloride or bromide, respectively, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch, wherein transcription of the fluorinase gene from the first promoter is induced in the presence of an inducer, wherein said riboswitch is responsive to said inducer;
wherein the cell is capable of expressing the fluorinase at least in the presence of said inducer.

Also provided is a cell capable of producing a fluorinated, a chlorinated or a brominated compound from a substrate in the presence of fluoride, chloride or bromide, respectively, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
wherein the cell is capable of expressing the activator of transcription and the fluorinase at least in the presence of said inducer.

Throughout the disclosure, a cell capable of producing a fluorinated, a chlorinated or a brominated compound from a substrate in the presence of fluoride, chloride or bromide, respectively, will refer to a cell capable of converting a substrate into a fluorinated, a chlorinated or a brominated product in the presence of fluoride, chloride or bromide, respectively.

Preferably, the cell can tolerate toxic compounds, such as fluorinated compounds. In some embodiments, the cell is a cell of a non-pathogenic organism. For example, the cell is a cell of a GRAS (Generally Regarded As Safe) organism.

Cells useful for performing the methods disclosed herein may be mammalian cells, plant cells, insect cells, yeast cells or bacterial cells.

In embodiments where the cell is a yeast cell, preferably the yeast is of the genus Saccharomyces, Pichia, Yarrowia, Kluyveromyces, Candida, Rhodotorula, Rhodosporidium, Cryptococcus, Trichosporon or Lipomyces. For example, the yeast is selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica.

In other embodiments, the cell is a bacterial cell of the Pseudomonas genus, the Bacillus genus, the Vibrio genus or the Escherichia genus. For example, the bacterial cell is selected from Pseudomonas putida, Pseudomonas fluorescens, Pseudomonas taiwanesis Pseudomonas syringae, Pseudomonas stutzeri, Pseudomonas oleovorans, Pseudomonas mendocina, Bacillus subtilis, Bacillus cereus, Bacillus megaterium, Vibrio natriegens and Escherichia coli. In preferred embodiments, the bacterial cell is a Pseudomonas putida cell, for example Pseudomonas putida KT2440.

The cell is used as production organism for fluorinated, chlorinated or brominated products as described herein.

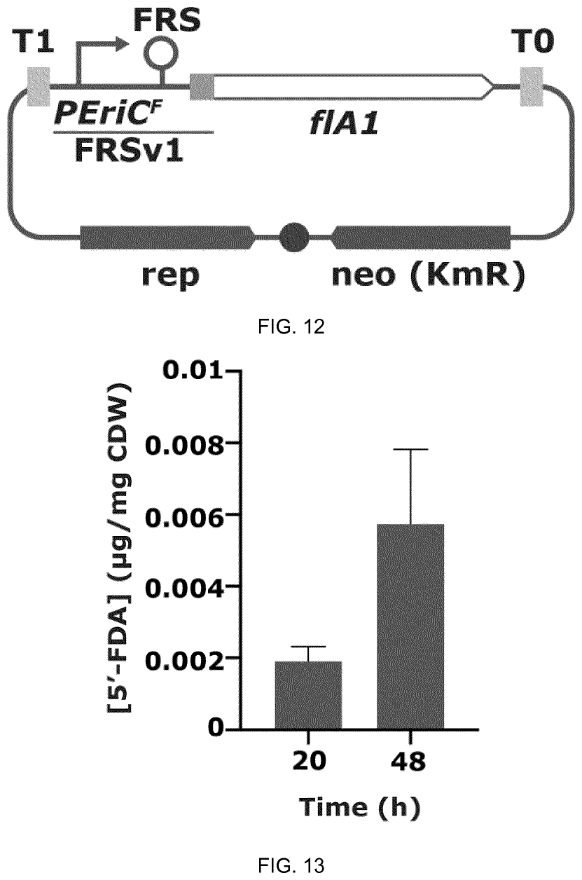

The cell comprises a first nucleic acid comprising a first promoter and a fluorinase gene encoding a fluorinase, and a second nucleic acid comprising a riboswitch. In some embodiments the first nucleic acid and the second nucleic acid are comprised within the same nucleic acid molecule. An example of such an embodiment is shown on FIG. 12.

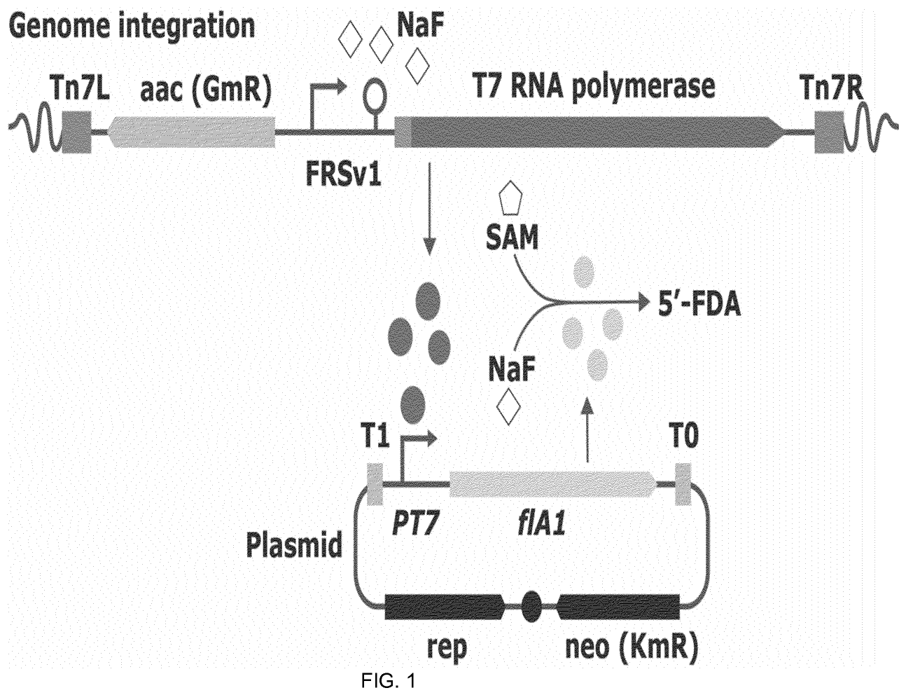

In other embodiments, the first nucleic acid and the second nucleic acid are different, i.e. they are not comprised within the same nucleic acid molecule. In such embodiments, the cell may comprise a first nucleic acid comprising a first promoter and a fluorinase gene encoding a fluorinase, and a second nucleic acid comprising a riboswitch, a second promoter and a gene encoding an activator of transcription. An example of such an embodiment is shown on FIG. 1.

### Fluorinase

The fluorinase gene encodes a fluorinase (EC 2.5.1.63). Fluorinases are enzymes which are capable of catalysing the formation of a C—F bond in the presence of fluoride. Fluorinases can also under some circumstances catalyse the formation of a C—Cl bond or of a C—Br bond. In the absence of fluoride, but in the presence of chloride, fluorinases can catalyse the formation of a C—Cl bond. In the absence of fluoride (and chloride) but in the presence of bromide, fluorinases can catalyse the formation of a C—Br bond.

Expression of the fluorinase gene is under the control of the first promoter comprised in the first nucleic acid. The fluorinase gene encodes a fluorinase or a functional variant thereof, such as a mutant, which retains the activity of the fluorinase but may have modified properties, such as modified substrate preferences, modified efficiency, and others. For example, fluorinases have been modified by directed evolution and rational design approaches to improve catalytic efficiency on non-native substrates (Sun et al., 2016; Thomsen et al., 2013). Such mutant fluorinases may also be used in the context of the present disclosure.

Fluorinases expressed in the cell of the present disclosure are capable of catalysing fluorination, chlorination or bromination of a substrate to obtain a fluorinated, a chlorinated or a brominated compound, respectively, as will be detailed further below. The fluorinase thus is capable of catalysing fluorination of a substrate to obtain a fluorinated compound.

In some embodiments, the fluorinase gene encodes the fluorinase of Streptomyces sp. MA37 as set forth in SEQ ID NO: 1 (FlA1), or a functional variant thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 1.

In some embodiments, the fluorinase gene encoding the fluorinase is as set forth in SEQ ID NO: 2, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 2.

As the fluorinase gene may be a heterologous gene, it may be codon-optimised to improve transcription in the cell in which it is to be expressed, as is known in the art.

Accordingly, in some embodiments, the fluorinase gene encoding the fluorinase is as set forth in SEQ ID NO: 3, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 3. SEQ ID NO: 3 encodes FlA1 of Streptomyces sp. MA37 (SEQ ID NO: 1) and has been codon optimised for expression in P. putida.

### Substrates

Fluorinase can convert S-adenosyl-L-methionine (SAM) to 5′-fluoro-5′-deoxyadenosine (5′-FDA) in the presence of inorganic fluoride (F⁻). The reaction is reversible. The enzyme can also convert other substrates to yield other products. For example, it can act on derivatives of SAM, such as a methylaza derivative, 5′-chloro-5′-deoxyadenosine, a 2-deoxyadenosine analogue, or L-methionine analogues, di-cyclic peptide conjugates of 5′-chlorodeoxy-2-ethynyladenosine, tri-cyclic peptide conjugates of 5′-chlorodeoxy-2-ethynyladenosine, fluoride, ¹⁸F, and chloride.

In some embodiments, the substrate is a SAM derivative of formula (I):

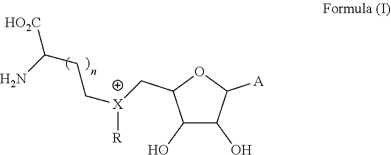

wherein:
n is 0, 1 or 2;
X is selected from the group consisting of: S, Se and NMe;
R is selected from the group consisting of: Me and propargyl;
A is a heterocycle.

The SAM derivative may thus be of formula (II):

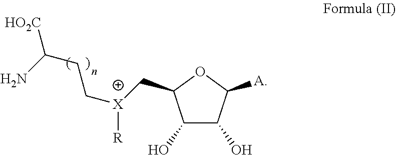

wherein X is selected from the group consisting of: S, Se and NMe; R is selected from the group consisting of: Me and propargyl; and A is a heterocycle.

In some embodiments, A is selected from the group consisting of

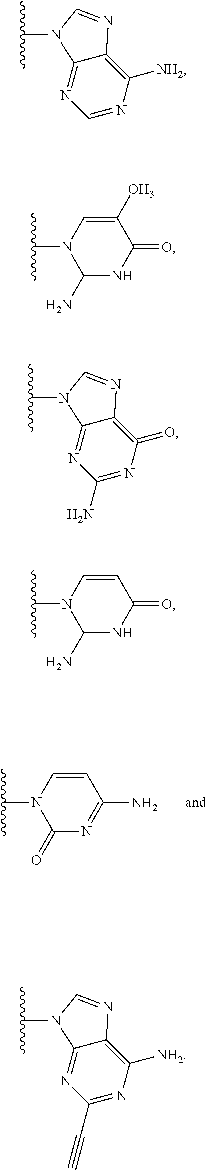

In some embodiments, the SAM derivative of formula (I) is of formula (III):

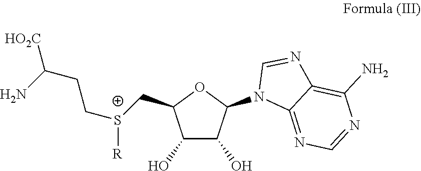

wherein R is selected from the group consisting of: Me and propargyl.

In preferred embodiments, the substrate is SAM.

In the presence of fluoride (F⁻) the fluorinase catalyses fluorination of the substrate. This is the preferred reaction catalysed by the enzyme. However, in the absence of fluoride, the enzyme is capable to catalyse other reactions. For example, in the absence of fluoride and in the presence of chloride, the enzyme is capable of chlorinating the substrates. In the absence of fluoride and chloride and in the presence of bromide, the enzyme is capable of brominating the substrates.

Hence, the cell disclosed herein which expresses a fluorinase can be used to obtain fluorinated compounds, chlorinated compounds or brominated compounds. The type of reaction can be directed by providing the appropriate co-substrates, fluoride, chloride or bromide.

The co-substrates may be provided in the form of salts. Preferred salts are soluble salts. In some embodiments, the reaction is a fluorination reaction and the co-substrate is a fluoride salt. Preferred fluoride salts are NaF or KF; less soluble salts such as CaF₂ may also be used. In other embodiments, the reaction is a chlorination reaction and the co-substrate is a chloride salt. Preferred chloride salts are NaCl or KCl; less soluble salts such as CaCl₂) may also be used. In other embodiments, the reaction is a bromination reaction and the co-substrate is a bromide salt. Preferred bromide salts are NaBr or KBr; less soluble salts such as CaBr₂ may also be used.

### Activator of Transcription and First Promoter

The cell may in some embodiments further comprise an activator of transcription, which can bind the first promoter. The first promoter controls expression of the fluorinase gene. In some embodiments, the activator of transcription is the T7 RNA polymerase and the first promoter is a T7 promoter, which is recognised by the T7 RNA polymerase. Accordingly, the fluorinase gene is expressed in the cell provided that the activator of transcription is expressed and active.

### Riboswitch and Second Promoter

The present inventors have found that in order to obtain tight and precise regulation of the expression of the fluorinase gene, it is advantageous to tightly regulate expression of the activator of transcription. This surprisingly results in an efficient system which can be tightly regulated to precisely control the fluorination, chlorination or bromination reaction. As shown in the examples, the use of a riboswitch together with a second promoter to control expression of the activator of transcription is particularly advantageous. However, the riboswitch may control expression of the fluorinase gene from the first promoter by activating transcription from the first promoter directly.

The riboswitch is activated by an inducer, as described herein below. In some embodiments, the inducer is a co-substrate of the reaction. For example, for fluorination of a substrate, the inducer is the co-substrate, i.e. fluoride. For chlorination of a substrate, the inducer may be the co-substrate chloride. For bromination of a substrate, the inducer may be the co-substrate bromide.

In other embodiments, the inducer is not a co-substrate of the reaction. For example, for chlorination of a substrate, the inducer is fluoride or bromide, preferably fluoride. For bromination of a substrate, the inducer is fluoride or chloride.

Preferably, a fluoride-responsive riboswitch (FRS) is used. For example, the FRS from Pseudomonas syringae is used. In its natural context this riboswitch drives the expression of the gene coding for the F⁻ transporter EriC^{F} in the presence of F⁻.

In embodiments where it is desirable to obtain fluorinated products, the riboswitch is fluoride-responsive, i.e. it is activated by the presence of fluoride. In embodiments where it is desirable to obtain chlorinated products, the riboswitch may be fluoride-responsive, i.e. activated by the presence of fluoride, and chloride is provided as co-substrate; or the riboswitch may be chloride-responsive, i.e. activated by the presence of chloride, and chloride is provided as inducer. In embodiments where it is desirable to obtain brominated products, the riboswitch may be fluoride-responsive, i.e. activated by the presence of fluoride, or it may be chloride-responsive, i.e. activated by the presence of chloride, and bromide is provided as co-substrate; or the riboswitch may be bromide-responsive, i.e. activated by the presence of bromide, and bromide is provided as inducer.

In some embodiments, the riboswitch is a fluoride-responsive riboswitch, such as the FRS riboswitch from P. syringae (SEQ ID NO: 4). In some embodiments, the cell is a Pseudomonas putida cell, in particular a P. putida KT2440 cell. In some embodiments, the activator of transcription is the T7 RNA polymerase and the first promoter is a T7 promoter.

In some embodiments, the second nucleic acid thus comprises a riboswitch having the sequence as set forth in SEQ ID NO: 4, or a functional variant thereof having at least 80% homology, such as at least 81% homology, such as at least 82% homology, such as at least 83% homology, such as at least 84% homology, such as at least 85% homology, such as at least 86% homology, such as at least 87% homology, such as at least 88% homology, such as at least 89% homology, such as at least 90% homology, such as at least 91%, such as at least 92%, such as at least 93%, such as at least 94%, such as at least 95%, such as at least 96%, such as at least 97%, such as at least 98%, such as at least 99% homology, such as 100% homology thereto. The term “functional variant” of an FRS refers to a sequence which retains the ability to drive expression of a gene downstream of it in response to fluoride.

The second nucleic acid in some embodiments may also comprise a second promoter. The second promoter may be the native promoter normally found with the riboswitch used. For example, the riboswitch is the FRS riboswitch from P. syringae as described above, and the second promoter is the native promoter of the eriC^{F} gene of P. syringae. In other embodiments, the second promoter is a constitutive promoter, such as a synthetic promoter, for example a PEM7 promoter, a PBG42 promoter, a PtetA promoter, or any constitutive promoter as known in the art.

Without being bound by theory, the use of a two-step regulation system, i.e. a second nucleic acid comprising a riboswitch and a promoter controlling expression of a first nucleic acid comprising a first promoter and a fluorinase gene, is expected to amplify the transcriptional signal and result in increased expression of the fluorinase gene in an inducible manner. However, as can be seen from the examples below, a one-step regulation system, i.e. wherein the riboswitch directly controls expression of the fluorinase from the first promoter, also leads to expression of fluorinase in an inducible manner.

In some embodiments, the second nucleic acid comprises or consists of the FRS sequence as set forth in SEQ ID NO: 4 or a functional variant thereof and of the native promoter of the eriC^{F} gene of P. syringae. The sequence of the corresponding second nucleic acid is set forth in SEQ ID NO: 6. In some embodiments, the second nucleic acid comprises or consists of SEQ ID NO: 6 or a functional variant thereof having at least 80% homology, such as at least 81% homology, such as at least 82% homology, such as at least 83% homology, such as at least 84% homology, such as at least 85% homology, such as at least 86% homology, such as at least 87% homology, such as at least 88% homology, such as at least 89% homology, such as at least 90% homology, such as at least 91%, such as at least 92%, such as at least 93%, such as at least 94%, such as at least 95%, such as at least 96%, such as at least 97%, such as at least 98%, such as at least 99%, such as 100% sequence homology thereto.

In other embodiments, the second nucleic acid comprises or consists of the FRS sequence as set forth in SEQ ID NO: 4 or a functional variant thereof and of the native promoter of the eriC^{F} gene of P. syringae, and further comprises one or more of the 5′-terminal codons of the eriC^{F} gene, such as one, two, three, four, five, six, seven, eight, nine or ten 5′-terminal codons of the eriC^{F} gene. In a particular embodiment, the second nucleic acid comprises or consists of the FRS sequence as set forth in SEQ ID NO: 4 or a functional variant thereof and of the native promoter of the eriC^{F} gene of P. syringae, and further comprises the eight 5′-terminal codons of the eriC^{F} gene. The corresponding sequence is as set forth in SEQ ID NO: 5. The second nucleic acid may comprise a sequence having at least 80% homology, such as at least 81% homology, such as at least 82% homology, such as at least 83% homology, such as at least 84% homology, such as at least 85% homology, such as at least 86% homology, such as at least 87% homology, such as at least 88% homology, such as at least 89% homology, such as at least 90% homology, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 5.

In some embodiments, the second nucleic acid comprises or consists of the FRS sequence as set forth in SEQ ID NO: 4 or a functional variant thereof and of the PEM7 promoter. The sequence of the corresponding second nucleic acid is set forth in SEQ ID NO: 8. In some embodiments, the second nucleic acid comprises or consists of SEQ ID NO: 8 or a homologue thereof having at least 80% homology, such as at least 81% homology, such as at least 82% homology, such as at least 83% homology, such as at least 84% homology, such as at least 85% homology, such as at least 86% homology, such as at least 87% homology, such as at least 88% homology, such as at least 89% homology, such as at least 90% homology, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 8.

In other embodiments, the second nucleic acid comprises or consists of the FRS sequence as set forth in SEQ ID NO: 4 or a functional variant thereof and of the PEM7 promoter, and further comprises one or more of the 5′-terminal codons of the eriC^{F} gene, such as one, two, three, four, five, six, seven, eight, nine or ten 5′-terminal codons of the eriC^{F} gene. In a particular embodiment, the second nucleic acid comprises the FRS sequence as set forth in SEQ ID NO: 4 or a functional variant thereof and of the PEM7 promoter, and further comprises the eight 5′-terminal codons of the eriC^{F} gene. The corresponding sequence is set forth in SEQ ID NO: 7. The second nucleic acid may thus have at least 80% homology to SEQ ID NO: 7, such as at least 81% homology, such as at least 82% homology, such as at least 83% homology, such as at least 84% homology, such as at least 85% homology, such as at least 86% homology, such as at least 87% homology, such as at least 88% homology, such as at least 89% homology, such as at least 90% homology, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 7.

Accordingly, in some embodiments the second nucleic acid is as set forth in SEQ ID NO: 5, SEQ ID NO: 6, SEQ ID NO: 7 or SEQ ID NO: 8, preferably as set forth in SEQ ID NO: 5, SEQ ID NO: 6 or SEQ ID NO: 8, most preferably as set forth in SEQ ID NO: 5 or SEQ ID NO: 6; or the nucleic acid comprises or consists of a sequence having at least 80% homology to SEQ ID NO: 5, SEQ ID NO: 6, SEQ ID NO: 7 or SEQ ID NO: 8, such as at least 81% homology, such as at least 82% homology, such as at least 83% homology, such as at least 84% homology, such as at least 85% homology, such as at least 86% homology, such as at least 87% homology, such as at least 88% homology, such as at least 89% homology, such as at least 90% homology, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 5, SEQ ID NO: 6, SEQ ID NO: 7 or SEQ ID NO: 8, preferably to SEQ ID NO: 5, SEQ ID NO: 6 or SEQ ID NO: 8, most preferably to SEQ ID NO: 5 or SEQ ID NO: 6.

In some embodiments, the second nucleic acid comprises or consists of SEQ ID NO: 5.

### Transporters

The intracellular availability of the co-substrate (fluoride, chloride or bromide) or inducer may have an influence on the efficiency of reaction. In some embodiments, it may thus be advantageous to use a cell which has reduced ability to transport the co-substrate out of the cell, in order to increase intracellular availability of the co-substrate.

Accordingly, in some embodiments, the cell further comprises a mutation in at least one fluoride, chloride or bromide transporter gene encoding a fluoride, chloride or bromide transporter, respectively, said mutation resulting in a partial or total loss of function of said fluoride, chloride or bromide transporter, respectively.

In embodiments where the cell is used to perform in vivo fluorination, the cell thus may comprise a mutation in at least one fluoride transporter gene encoding a fluoride transporter, said mutation resulting in a partial or total loss of function of said fluoride transporter.

In embodiments where the cell is used to perform in vivo chlorination, the cell thus may comprise a mutation in at least one chloride transporter gene encoding a chloride transporter, said mutation resulting in a partial or total loss of function of said chloride transporter.

In embodiments where the cell is used to perform in vivo bromination, the cell thus may comprise a mutation in at least one bromide transporter gene encoding a bromide transporter, said mutation resulting in a partial or total loss of function of said bromide transporter.

The mutation may be a partial or total deletion, as is known in the art.

For example, in a specific embodiment, the cell is a Pseudomonas putida cell used for in vivo fluorination of a substrate as described herein, in particular a P. putida KT2440 cell, and the cell has a mutation in the crcB gene as set forth in SEQ ID NO: 9. In one embodiment, the crcB gene has been deleted.

In specific embodiments, the cell is capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In another embodiment, the cell is capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter preferably is the native promoter of the eriC^{F} gene of P. syringae or the PEM7 promoter;
b) a second nucleic acid comprising a fluoride-responsive riboswitch such as the FRS riboswitch from P. syringae (SEQ ID NO: 4), wherein transcription of the fluorinase gene can be induced by fluoride;
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing a chlorinated compound from a substrate in the presence of chloride, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, wherein the activator of transcription is the T7 RNA polymerase, said gene being under the control of the second promoter, wherein transcription of the T7 RNA polymerase can be induced by fluoride, chloride or bromide, wherein T7 RNA polymerase upon expression activates transcription from the T7 promoter;
   wherein the cell is capable of expressing the activator of transcription and the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter and/or a mutation of a gene encoding chloride transporter resulting in a partial or total loss of activity of the chloride transporter.

In other embodiments, the cell is capable of producing a brominated compound from a substrate in the presence of bromide, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, wherein the activator of transcription is the T7 RNA polymerase, said gene being under the control of the second promoter, wherein transcription of the T7 RNA polymerase can be induced by fluoride, chloride or bromide, wherein T7 RNA polymerase upon expression activates transcription from the T7 promoter;
   wherein the cell is capable of expressing the activator of transcription and the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter and/or a mutation of a gene encoding bromide transporter resulting in a partial or total loss of activity of the bromide transporter.

In specific embodiments, the cell is capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter is preferably the promoter of the eriC^{F} gene or the PEM7 promoter;
b) a second nucleic acid comprising a riboswitch,
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. In some embodiments the first nucleic acid and the second nucleic acid are the same nucleic acid, and transcription from the first promoter is induced by the riboswitch in the presence of inducer.

In other embodiments, the cell is capable of producing a chlorinated compound from a substrate in the presence of chloride, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having a least 80% homology thereto, and wherein the first promoter is preferably the promoter of the eriC^{F} gene or the PEM7 promoter;
b) a second nucleic acid comprising a riboswitch,
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. In some embodiments the first nucleic acid and the second nucleic acid are the same nucleic acid, and transcription from the first promoter is induced by the riboswitch in the presence of inducer.

In other embodiments, the cell is capable of producing a brominated compound from a substrate in the presence of bromide, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter is preferably the promoter of the eriC^{F} gene or the PEM7 promoter;
b) a second nucleic acid comprising a riboswitch,
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. In some embodiments the first nucleic acid and the second nucleic acid are the same nucleic acid, and transcription from the first promoter is induced by the riboswitch in the presence of inducer.

### Phosphorylase

In some embodiments, the cell may further express a phosphorylase (EC 2.4.2.28), and is thus capable of producing a phosphorylated compound from a fluorinated, chlorinated or brominated compound. In some embodiments, the cell is capable of producing a phosphorylated fluorinated, chlorinated or brominated compound from a substrate in the presence of fluoride.

In specific embodiments, the cell is capable of phosphorylating a fluorinated compound in the presence of fluoride, said cell comprising:
a) a first nucleic acid comprising a first promoter and a gene encoding a phosphorylase (EC 2.4.2.28) such as FlB1 or DeoD, said phosphorylase being capable of catalysing the phosphorylation of a fluorinated adenosine, said phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the phosphorylase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. The cell is thus capable of producing a phosphorylated, fluorinated compound from a fluorinated compound. The fluorinated compound may be produced by the cell as described herein above, or it may be provided to the cell, e.g. in the medium.

In other embodiments, the cell is capable of phosphorylating a chlorinated compound in the presence of chloride, said cell comprising:
a) a first nucleic acid comprising a first promoter and a gene encoding a phosphorylase (EC 2.4.2.28) such as FlB1 or DeoD, said phosphorylase being capable of catalysing the phosphorylation of a chlorinated adenosine, said phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the phosphorylase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. The cell is thus capable of producing a phosphorylated, chlorinated compound from a chlorinated compound. The chlorinated compound may be produced by the cell as described herein above, or it may be provided to the cell, e.g. in the medium.

In other embodiments, the cell is capable of phosphorylating a brominated compound in the presence of bromide, said cell comprising:
a) a first nucleic acid comprising a first promoter and a gene encoding a phosphorylase (EC 2.4.2.28) such as FlB1 or DeoD, said phosphorylase being capable of catalysing the phosphorylation of a brominated adenosine, said phosphorylase gene being under control of the first promoter, wherein the first promoter is preferably a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the phosphorylase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. The cell is thus capable of producing a phosphorylated, brominated compound from a brominated compound. The brominated compound may be produced by the cell as described herein above, or it may be provided to the cell, e.g. in the medium.

In some embodiments, the cell is capable of producing a fluorinated, a chlorinated or a brominated compound and is further capable of phosphorylating said compound. In some embodiments, the phosphorylase gene and the fluorinase gene are both controlled by the riboswitch. In other words, the phosphorylase and the fluorinase gene are present within a same nucleic acid molecule (here the first nucleic acid), for example a vector.

In some embodiments, the cell is capable of producing a fluorinated and phosphorylated compound from a substrate in the presence of fluoride, said cell comprising:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28) such as an flB1 gene or a DeoD gene, said phosphorylase being capable of catalysing the phosphorylation of a fluorinated adenosine, said fluorinase gene and said phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4),
   wherein the cell is capable of expressing the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing a chlorinated and phosphorylated compound from a substrate in the presence of chloride, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as an flB1 gene or a deoD gene, said phosphorylase being capable of catalysing the phosphorylation of a fluorinated, chlorinated and/or brominated adenosine, said fluorinase gene and said phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4);
   wherein the cell is capable of expressing the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing a brominated and phosphorylated compound from a substrate in the presence of bromide, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as an flB1 gene or a deoD gene, said phosphorylase being capable of catalysing the phosphorylation of a fluorinated, chlorinated and/or brominated adenosine, said fluorinase gene and phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4);
   wherein the cell is capable of expressing the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In specific embodiments, the cell is capable of producing a fluorinated and phosphorylated compound from a substrate in the presence of fluoride, said cell comprising:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28) such as an flB1 gene or a deoD gene, said phosphorylase being capable of catalysing the phosphorylation of a fluorinated adenosine, said fluorinase gene and said phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing a chlorinated and phosphorylated compound from a substrate in the presence of chloride, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as an flB1 gene or a deoD gene, said phosphorylase being capable of catalysing the phosphorylation of a fluorinated, chlorinated and/or brominated adenosine, said fluorinase gene and said phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the phosphorylase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing a brominated and phosphorylated compound from a substrate in the presence of bromide, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as an flB1 gene or a deoD gene, said phosphorylase being capable of catalysing the phosphorylation of a fluorinated, chlorinated and/or brominated adenosine, said fluorinase gene and phosphorylase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the phosphorylase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In some embodiments, the phosphorylase gene is the flB1 gene from Streptomyces sp. MA37 as set forth in SEQ ID NO: 12 or SEQ ID NO: 13, encoding the FlB1 phosphorylase as set forth in SEQ ID NO: 11. In some embodiments, the phosphorylase gene is a homologue of flB1 having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 12 or SEQ ID NO: 13.

In some embodiments, the phosphorylase is a functional variant of FlB1 as set forth in SEQ ID NO: 11, such as a functional variant having at least 80% homology, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 11.

In some embodiments, the phosphorylase gene is the deoD gene from Escherichia coli as set forth in SEQ ID NO: 14, encoding the DeoD phosphorylase as set forth in SEQ ID NO: 15. In some embodiments, the phosphorylase gene is a homologue of deoD having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 14.

In some embodiments, the phosphorylase is a functional variant of DeoD as set forth in SEQ ID NO: 15, such as a functional variant having at least 80% homology, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 15.

The phosphorylase gene, such as the flB1 gene or the deoD gene, may be codon-optimised as is known in the art.

Thus in embodiments where the cell is capable of expressing a phosphorylase as described above, such as an FlB1 phosphorylase or a DeoD phosphorylase, the cell is capable of converting 5′-fluoro-5′-deoxyadenosine to 5′-deoxy-5′-fluoro-D-ribose-1-phosphate. The cell is thus capable of producing 5′-deoxy-5′-fluoro-D-ribose-1-phosphate.

### Nucleosidase

In some embodiments, the cell may further express a nucleosidase (EC 3.2.2.9), and is thus capable of producing a deoxyribose compound from a fluorinated, chlorinated or brominated compound. The nucleosidase catalyses the reaction:
S-adenosyl-L-homocysteine+H₂O<=>S-(5′-deoxy-D-ribos-5-yl)-L-homocysteine+adenine

In some embodiments, the cell is capable of producing a 5′-fluorodeoxyribose, a 5′-chlorodeoxyribose or a 5′-bromodeoxyribose from a substrate in the presence of fluoride.

In specific embodiments, the cell is capable of producing 5′-fluorodeoxyribose in the presence of fluoride, said cell comprising:
a) a first nucleic acid comprising a first promoter and a gene encoding a nucleosidase (EC 3.2.2.9) such as Pfs, said nucleosidase being capable of catalysing the conversion of 5′-fluoro-5′-deoxyadenosine to 5′-fluorodeoxyribose, said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the nucleosidase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. The cell is thus capable of converting 5′-fluoro-5′-deoxyadenosine to 5′-fluorodeoxyribose, and can produce 5′-fluorodeoxyribose. The 5′-fluoro-5′-deoxyadenosine may be produced by the cell as described herein above, or it may be provided to the cell, e.g. in the medium.

In other embodiments, the cell is capable of producing 5′-chlorodeoxyribose in the presence of chloride, said cell comprising:
a) a first nucleic acid comprising a first promoter and a gene encoding a nucleosidase (EC 3.2.2.9) such as Pfs, said nucleosidase being capable of catalysing the conversion of 5′-chloro-5′-deoxyadenosine to 5′-chlorodeoxyribose, said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the nucleosidase gene at least in the presence of chloride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. The cell is thus capable of converting 5′-chloro-5′-deoxyadenosine to 5′-chlorodeoxyribose, and is capable of producing 5′-chlorodeoxyribose. The 5′-chloro-5′-deoxyadenosine may be produced by the cell as described herein above, or it may be provided to the cell, e.g. in the medium.

In other embodiments, the cell is capable of producing 5′-bromodeoxyribose in the presence of bromide, said cell comprising:
a) a first nucleic acid comprising a first promoter and a gene encoding a nucleosidase (EC 3.2.2.9) such as Pfs, said nucleosidase being capable of catalysing the conversion of 5′-bromo-5′-deoxyadenosine to 5′-bromodeoxyribose, said nucleosidase gene being under control of the first promoter, wherein the first promoter is preferably a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription is the T7 RNA polymerase, said gene being under the control of the second promoter, wherein transcription of the T7 RNA 5′-polymerase can be induced by fluoride, wherein T7 RNA polymerase upon expression activates transcription from the T7 promoter;
   wherein the cell is capable of expressing the activator of transcription and the nucleosidase gene at least in the presence of bromide. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. The cell is thus capable of converting 5′-bromo-5′-deoxyadenosine to 5′-bromodeoxyribose and can produce 5′-bromodeoxyribose. The 5′-bromo-5′-deoxyadenosine may be produced by the cell as described herein above, or it may be provided to the cell, e.g. in the medium.

In some embodiments, the cell is capable of producing a fluorinated, a chlorinated or a brominated compound and is further capable of converting said compound into a 5′-fluorodeoxyribose, a 5′-chlorodeoxyribose or a 5′-bromodeoxyribose. In some embodiments, the nucleosidase gene and the fluorinase gene are both controlled by the riboswitch. In other words, the nucleosidase and the fluorinase gene are present within a same nucleic acid molecule (here the first nucleic acid), for example a vector.

In some embodiments, the cell is capable of producing 5′-fluorodeoxyribose in the presence of fluoride, said cell comprising:
a) a first nucleic acid comprising a first promoter, a gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, said fluorinase gene and said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), wherein the cell is capable of expressing the fluorinase and the nucleosidase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing 5′-chlorodeoxyribose in the presence of chloride, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-chloro-5′-deoxyadenosine to 5′-chlorodeoxyribose, said fluorinase gene and said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4);
   wherein the cell is capable of expressing the fluorinase and the nucleosidase at least in the presence of chloride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing 5′-bromo-5′-deoxyadenosine to 5′-bromodeoxyribose in the presence of bromide, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-bromo-5′-deoxyadenosine to 5′-bromodeoxyribose, said fluorinase gene and nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4);
   wherein the cell is capable of expressing the fluorinase and the nucleosidase at least in the presence of bromide. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In specific embodiments, the cell is capable of producing 5′-fluorodeoxyribose in the presence of fluoride, said cell comprising:
a) a first nucleic acid comprising a first promoter, a fluorinase gene encoding a fluorinase (EC 2.5.1.63) capable of catalysing the formation of a C—F bond, and and a gene encoding a nucleosidase (EC 3.2.2.9) such as Pfs, said nucleosidase being capable of catalysing the conversion of 5′-fluoro-5′-deoxyadenosine to 5′-fluorodeoxyribose, said fluorinase gene and said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the nucleosidase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of 5′-chloro-5′-deoxyadenosine to 5′-chlorodeoxyribose in the presence of chloride, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-chloro-5′-deoxyadenosine to 5′-chlorodeoxyribose, said fluorinase gene and said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the nucleosidase gene at least in the presence of chloride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In other embodiments, the cell is capable of producing 5′-bromodeoxyribose in the presence of bromide, said cell comprising:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-bromo-5′-deoxyadenosine to 5′-bromodeoxyribose, said fluorinase gene and nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the nucleosidase gene at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In some embodiments, the nucleosidase gene is the pfs gene from Escherichia coli as set forth in SEQ ID NO: 16, encoding the Pfs nucleosidase as set forth in SEQ ID NO: 17. In some embodiments, the nucleosidase gene is a homologue of pfs having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 16.

In some embodiments, the nucleosidase is a functional variant of Pfs as set forth in SEQ ID NO: 17, such as a functional variant having at least 80% homology, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 17.

The nucleosidase gene, such as the pfs gene, may be codon-optimised as is known in the art.

Thus in embodiments where the cell is capable of expressing a nucleosidase as described above, such as Pfs nucleosidase, the cell is capable of converting 5′-fluoro-5′-deoxyadenosine to 5′-fluorodeoxyribose. The cell is thus capable of producing 5′-fluorodeoxyribose.

### Expression System

The present disclosure further relates to an expression system for expression in a cell, said system comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch,
   wherein transcription of the fluorinase gene from the first promoter is induced in the presence of an inducer, wherein said riboswitch is responsive to said inducer.

In some embodiments, the disclosure further relates to an expression system for expression in a cell, said system comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter.

The elements of the expression system, i.e. the first nucleic acid, the first promoter, the fluorinase gene encoding a fluorinase, the second nucleic acid, the riboswitch, the second promoter and the gene encoding an activator of transcription, may be as described herein.

The expression system is suitable for expression of the first nucleic acid and of the second nucleic acid in a cell, where the cell may be as described herein. In particular embodiments, the cell is a Pseudomonas cell, such as a Pseudomonas putida cell, such as a Pseudomonas putida KT2440 cell.

The first and the second nucleic acids may be independently comprised in a vector or integrated in the genome of the cell. In some embodiments, the first and the second nucleic acids are provided in a vector, such as in one vector. In some embodiments, the first nucleic acid is provided in a vector, and the second nucleic acid is provided in another vector. In some embodiments, the first nucleic acid is provided in a vector and the second nucleic acid is integrated in the genome of the cell. In some embodiments, the second nucleic acid is provided in a vector and the first nucleic acid is integrated in the genome of the cell. In some embodiments, the first and the second nucleic acids are integrated in the genome of the cell.

In some embodiments, the second nucleic acid comprises a riboswitch, and the second nucleic acid and the first nucleic acid are the same. Thus the present disclosure further relates to an expression system for expression in a cell, said system comprising:
a) a first nucleic acid comprising a first promoter and a fluorinase gene encoding a fluorinase (EC 2.5.1.63) capable of catalysing the formation of a C—F bond, said fluorinase gene being under the control of the first promoter and wherein the transcription of the fluorinase gene can be induced by an inducer;
b) a second nucleic acid comprising a riboswitch.

The elements of the expression system, i.e. the first nucleic acid, the first promoter, the fluorinase gene encoding a fluorinase, the second nucleic acid and the riboswitch may be as described herein.

The expression system is suitable for expression in a cell where the cell may be as described herein. In particular embodiments, the cell is a Pseudomonas cell, such as a Pseudomonas putida cell, such as a Pseudomonas putida KT2440 cell.

The first nucleic acid may be comprised in a vector or integrated in the genome of the cell. In some embodiments, the first nucleic acid is provided in a vector. In some embodiments, the first nucleic acid is integrated in the genome of the cell.

The present disclosure further relates to an expression system for expression in a cell, said system comprising:
a) a first nucleic acid comprising a first promoter and a gene encoding a phosphorylase (EC 2.4.2.28) such as FlB1 or DeoD, said phosphorylase being capable of catalysing the phosphorylation of a fluorinated, chlorinated and/or brominated adenosine, said phosphorylase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter.

The above expression system is thus suitable for phosphorylating a fluorinated, a chlorinated or a brominated compound as described herein. The elements of the expression system, i.e. the first nucleic acid, the first promoter, the gene encoding a phosphorylase, the second nucleic acid, the riboswitch, the second promoter, and the gene encoding an activator of transcription, may be as described herein.

The present disclosure further relates to an expression system for expression in a cell, said system comprising:
a) a first nucleic acid comprising a first promoter and a gene encoding a nucleosidase (EC 3.2.2.9) such as Pfs, said nucleosidase being capable of catalysing the conversion of 5′-fluoro-5′-deoxyadenosine to 5′-fluorodeoxyribose, and/or conversion of 5′-chlorod-5′-eoxyadenosine to 5′-chlorodeoxyribose and/or conversion of 5′-bromo-5′-deoxyadenosine to 5′-bromodeoxyribose, said nucleosidase gene being under control of the first promoter;
b) a second nucleic acid comprising a riboswitch, a second promoter and a gene encoding an activator of transcription, said gene being under the control of the second promoter,
   wherein transcription of the activator of transcription can be induced by an inducer, wherein the activator of transcription upon expression activates transcription from the first promoter.

The above expression system is thus suitable for producing a 5′-fluorodeoxyribose, a 5′-chlorodeoxyribose or a 5′-bromodeoxyribose as described herein. The elements of the expression system, i.e. the first nucleic acid, the first promoter, the gene encoding a nucleosidase, the second nucleic acid, the riboswitch, the second promoter, and the gene encoding an activator of transcription, may be as described herein.

The expression system is suitable for expression of the first nucleic acid and of the second nucleic acid in a cell, where the cell may be as described herein. In particular embodiments, the cell is a Pseudomonas cell, such as a Pseudomonas putida cell, such as a Pseudomonas putida KT2440 cell.

The first and the second nucleic acids may be independently comprised in a vector or integrated in the genome of the cell. In some embodiments, the first and the second nucleic acids are provided in a vector, such as in one vector. In some embodiments, the first nucleic acid is provided in a vector, and the second nucleic acid is provided in another vector. In some embodiments, the first nucleic acid is provided in a vector and the second nucleic acid is integrated in the genome of the cell. In some embodiments, the second nucleic acid is provided in a vector and the first nucleic acid is integrated in the genome of the cell. In some embodiments, the first and the second nucleic acids are integrated in the genome of the cell.

The present disclosure further relates to an expression system for expression in a cell, said system comprising:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28) such as FlB1 or DeoD, said phosphorylase being capable of catalysing the phosphorylation of a fluorinated, chlorinated and/or brominated adenosine, said fluorinase gene and phosphorylase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter.

In other embodiments, the expression system comprises:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28) such as FlB1 or DeoD, said phosphorylase being capable of catalysing the phosphorylation of a fluorinated, chlorinated and/or brominated adenosine, said fluorinase gene and phosphorylase gene being under control of the first promoter;
b) a second nucleic acid comprising a riboswitch.

Such expression systems comprising both a fluorinase gene and a gene encoding a phosphorylase when introduced in the cell allow fluorination and phosphorylation of compounds in the presence of inducer. The elements of the expression system, i.e. the first nucleic acid, the first promoter, the fluorinase gene encoding a fluorinase, and the phosphorylase gene encoding a phosphorylase, the second nucleic acid, the riboswitch, the second promoter and the gene encoding an activator of transcription, may be as described herein.

The present disclosure further relates to an expression system for expression in a cell, said system comprising:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, and/or conversion of 5′-chloro-5′-deoxyadenosine to 5′-chlorodeoxyribose and/or conversion of 5′-bromo-5′-deoxyadenosine to 5′-bromodeoxyribose, said fluorinase gene and nucleosidase gene being under control of the first promoter;
b) a second nucleic acid comprising a riboswitch, a second promoter and a gene encoding an activator of transcription, said gene being under the control of the second promoter,
   wherein transcription of the activator of transcription can be induced by an inducer, wherein the activator of transcription upon expression activates transcription from the first promoter.

In other embodiments, the expression system comprises:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, and/or conversion of 5′-chloro-5′-deoxyadenosine to 5′-chlorodeoxyribose and/or conversion of 5′-bromo-5′-deoxyadenosine to 5′-bromodeoxyribose, said fluorinase gene and nucleosidase gene being under control of the first promoter;
b) a second nucleic acid comprising a riboswitch.

Such expression systems comprising both a fluorinase gene and a gene encoding a nucleosidase when introduced in the cell allow production of a 5′-fluorodeoxyribose, a 5′-chlorodeoxyribose or a 5′-bromodeoxyribose in the presence of inducer. The elements of the expression system, i.e. the first nucleic acid, the first promoter, the fluorinase gene encoding a fluorinase, and the nucleosidase gene encoding a nucleosidase, the second nucleic acid, the riboswitch, the second promoter and the gene encoding an activator of transcription, may be as described herein.

The expression system is suitable for expression of the first nucleic acid and of the second nucleic acid in a cell, where the cell may be as described herein. In particular embodiments, the cell is a Pseudomonas cell, such as a Pseudomonas putida cell, such as a Pseudomonas putida KT2440 cell.

The first and the second nucleic acids may be independently comprised in a vector or integrated in the genome of the cell. In some embodiments, the first and the second nucleic acids are provided in a vector, such as in one vector. In some embodiments, the first nucleic acid is provided in a vector, and the second nucleic acid is provided in another vector. In some embodiments, the first nucleic acid is provided in a vector and the second nucleic acid is integrated in the genome of the cell. In some embodiments, the second nucleic acid is provided in a vector and the first nucleic acid is integrated in the genome of the cell. In some embodiments, the first and the second nucleic acids are integrated in the genome of the cell.

Methods for introducing vectors in the cell and methods for integrating nucleic acids in the genome of a cell are known in the art.

### Method for In Vivo Fluorination, Chlorination or Bromination of a Substrate

Also provided herein is a method for in vivo fluorination, chlorination or bromination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
   b) a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate selected from a fluoride salt, a chloride salt or a bromide salt,
   whereby transcription of the gene encoding an activator of transcription is induced,
   thereby inducing transcription of the fluorinase gene,
      thereby inducing fluorination, chlorination or bromination of the substrate to yield a fluorinated, a chlorinated or a brominated product, respectively.

In one embodiment, the method is a method for in vivo fluorination, chlorination or bromination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter and wherein the transcription of the fluorinase gene can be induced by an inducer;
   b) a second nucleic acid comprising a riboswitch,
ii) adding the inducer to the medium and incubating the cell in the presence of inducer and a co-substrate selected from a fluoride salt, a chloride salt or a bromide salt, whereby transcription of the fluorinase gene is induced,
   thereby inducing fluorination, chlorination or bromination of the substrate to
   yield a fluorinated, a chlorinated or a brominated product, respectively.

In this embodiment, the first nucleic acid and the second nucleic acid may be the same molecule, i.e. the riboswitch controls transcription from the first promoter directly.

In other embodiments, the method is a method for producing a phosphorylated and fluorinated, chlorinated or brominated compound, and the expression system comprises a gene encoding a phosphorylase (EC 2.4.2.28).

In one such embodiment, the method is a method for in vivo phosphorylation of a fluorinated, chlorinated, or brominated substrate, comprising the steps of:
i) propagating a cell in a medium, said cell comprising:
   a) a first nucleic acid comprising a first promoter and a gene encoding a phosphorylase (EC 2.4.2.28) such as FlB1 or DeoD, said phosphorylase being capable of catalysing the phosphorylation of a fluorinated, chlorinated and/or brominated substrate, said phosphorylase gene being under control of the first promoter;
   b) a second nucleic acid comprising a riboswitch,
ii) adding the inducer to the medium and incubating the cell in the presence of inducer,
   thereby inducing transcription of the phosphorylase gene,
   thereby inducing phosphorylation of the fluorinated, the chlorinated and/or the brominated substrate to yield a fluorinated and phosphorylated product, a chlorinated and phosphorylated product, or a brominated and phosphorylated product, respectively.

In another such embodiment, the method is a method for in vivo phosphorylation of a fluorinated, chlorinated, or brominated substrate, comprising the steps of:
i) propagating a cell in a medium, said cell comprising:
   a) a first nucleic acid comprising a first promoter and a gene encoding a phosphorylase (EC 2.4.2.28) such as FlB1 or DeoD, said phosphorylase being capable of catalysing the phosphorylation of a fluorinated, chlorinated and/or brominated adenosine, said phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter
ii) adding the inducer to the medium and incubating the cell in the presence of inducer,
   whereby transcription of the gene encoding an activator of transcription is induced,
   thereby inducing transcription of the phosphorylase gene,
   thereby inducing phosphorylation of the fluorinated, chlorinated and/or brominated substrate to yield a fluorinated and phosphorylated product, a chlorinated and phosphorylated product, or a brominated and phosphorylated product, respectively.

In one embodiment, the method is a method for in vivo fluorination and phosphorylation, chlorination and phosphorylation or bromination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as FlB1 or DeoD, said phosphorylase being capable of catalysing the phosphorylation of a fluorinated, chlorinated and/or brominated adenosine, said fluorinase gene and phosphorylase gene being under control of the first promoter;
   b) a second nucleic acid comprising a riboswitch;
ii) adding the inducer to the medium and incubating the cell in the presence of inducer and a co-substrate selected from a fluoride salt, a chloride salt or a bromide salt,
   whereby transcription of the genes encoding the fluorinase and the phosphorylase is induced,
      thereby inducing fluorination, chlorination or bromination and phosphorylation of the substrate to yield a fluorinated and phosphorylated, a chlorinated and phosphorylated or a brominated and phosphorylated product, respectively.

In one embodiment, the method is a method for in vivo fluorination and phosphorylation, chlorination and phosphorylation or bromination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as FlB1 or DeoD, said phosphorylase being capable of catalysing the phosphorylation of a fluorinated, chlorinated and/or brominated adenosine, said fluorinase gene and phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate selected from a fluoride salt, a chloride salt or a bromide salt,
   whereby transcription of the gene encoding an activator of transcription is induced,
   thereby inducing transcription of the fluorinase gene and the phosphorylase gene,
   thereby inducing fluorination, chlorination or bromination and phosphorylation of the substrate to yield a fluorinated and phosphorylated, a chlorinated and phosphorylated or a brominated and phosphorylated product, respectively.

In some embodiments, the inducer and the co-substrate are identical.

In some embodiments, the fluorinated, the chlorinated or the brominated substrate such as a fluorinated adenosine, a chlorinated adenosine or a brominated adenosine, are provided in the medium. In other embodiments, the cell is capable of synthesising the fluorinated, the chlorinated or the brominated substrate which is to be phosphorylated.

In other embodiments, the method is a method for producing a 5′-fluorodeoxyribose, a 5′-chlorodeoxyribose or a 5′-bromodeoxyribose, and the expression system comprises a gene encoding a nucleosidase (EC 3.2.2.9).

In one such embodiment, the method is a method for in vivo production of 5′-fluorodeoxyribose, 5′-chlorodeoxyribose and/or 5′-bromodeoxyribose, comprising the steps of:
i) propagating a cell in a medium, said cell comprising:
   a) a first nucleic acid comprising a first promoter and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said nucleosidase gene being under control of the first promoter;
   b) a second nucleic acid comprising a riboswitch,
ii) adding the inducer to the medium and incubating the cell in the presence of inducer,
   whereby transcription of the gene encoding an activator of transcription is induced,
   thereby inducing the production of 5′-fluoro-5′-deoxyadenosine and its conversion to 5′-fluorodeoxyribose, the production of 5′-chloro-5′-deoxyadenosine and its conversion to 5′-chlorodeoxyribose and/or the production of 5′-bromo-5′-deoxyadenosine and its conversion to 5′-bromodeoxyribose.

In another such embodiment, the method is a method for the production of 5′-fluorodeoxyribose, 5′-chlorodeoxyribose and/or 5′-bromodeoxyribose, comprising the steps of:
i) propagating a cell in a medium, said cell comprising:
   a) a first nucleic acid comprising a first promoter and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said nucleosidase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter
ii) adding the inducer to the medium and incubating the cell in the presence of inducer,
   whereby transcription of the gene encoding an activator of transcription is induced,
   thereby inducing transcription of the nucleosidase gene,
   thereby inducing the production of 5′-fluoro-5′-deoxyadenosine and its conversion to 5′-fluorodeoxyribose, the production of 5′-chloro-5′-deoxyadenosine and its conversion to 5′-chlorodeoxyribose and/or the production of 5′-bromo-5′-deoxyadenosine and its conversion to 5′-bromodeoxyribose.

In one embodiment, the method is a method for in vivo production of 5′-fluorodeoxyribose, 5′-chlorodeoxyribose and/or 5′-bromodeoxyribose, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said fluorinase gene and nucleosidase gene being under control of the first promoter;
   b) a second nucleic acid comprising a riboswitch;
ii) adding the inducer to the medium and incubating the cell in the presence of inducer and a co-substrate selected from a fluoride salt, a chloride salt or a bromide salt,
   whereby transcription of the genes encoding the fluorinase and the nucleosidase is induced,
      thereby inducing the production of 5′-fluoro-5′-deoxyadenosine and its conversion to 5′-fluorodeoxyribose, the production of 5′-chloro-5′-deoxyadenosine and its conversion to 5′-chlorodeoxyribose and/or the production of 5′-bromo-5′-deoxyadenosine and its conversion to 5′-bromodeoxyribose.

In one embodiment, the method is a method for in vivo production of 5′-fluorodeoxyribose, 5′-chlorodeoxyribose and/or 5′-bromodeoxyribose, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said fluorinase gene and nucleosidase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate selected from a fluoride salt, a chloride salt or a bromide salt,
   whereby transcription of the gene encoding an activator of transcription is induced,
   thereby inducing transcription of the fluorinase gene and the nucleosidase gene,
      thereby inducing the production of 5′-fluoro-5′-deoxyadenosine and its conversion to 5′-fluorodeoxyribose, the production of 5′-chloro-5′-deoxyadenosine and its conversion to 5′-chlorodeoxyribose and/or the production of 5′-bromo-5′-deoxyadenosine and its conversion to 5′-bromodeoxyribose.

In preferred embodiments, the method is for producing 5′-fluoro-5′-deoxyadenosine and converting it to 5′-fluorodeoxyribose.

In some embodiments, the inducer and the co-substrate are identical.

In one embodiment, the method is a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a fluoride salt,
   whereby transcription of the gene encoding an activator of transcription is induced,
   thereby inducing transcription of the fluorinase gene,
      thereby inducing fluorination of the substrate to yield a fluorinated product.

In one embodiment, the method is a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter and wherein the transcription of the fluorinase gene can be induced by an inducer;
   b) a second nucleic acid comprising a riboswitch,
ii) adding the inducer to the medium and incubating the cell in the presence of inducer and a fluoride salt,
   whereby transcription of the fluorinase gene is induced, thereby inducing fluorination of the substrate to yield a fluorinated product.

In one embodiment, the method is a method for in vivo phosphorylation of a fluorinated substrate, comprising the steps of:
i) propagating a cell in a medium, said cell comprising:
   a) a first nucleic acid comprising a first promoter and a gene encoding a phosphorylase (EC 2.4.2.28) such as FlB1 or DeoD, said phosphorylase being capable of catalysing the phosphorylation of the fluorinated substrate, said phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter
ii) adding the inducer to the medium and incubating the cell in the presence of inducer,
   whereby transcription of the gene encoding an activator of transcription is induced,
   thereby inducing transcription of the phosphorylase gene,
   thereby inducing phosphorylation of the fluorinated substrate to yield a fluorinated and phosphorylated product.

In one embodiment, the method is a method for in vivo fluorination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28) such as FlB1 or DeoD, said phosphorylase being capable of catalysing the phosphorylation of a fluorinated substrate, said fluorinase gene and phosphorylase gene being under control of the first promoter;
   b) a second nucleic acid comprising a riboswitch;
ii) adding the inducer to the medium and incubating the cell in the presence of inducer and a fluoride salt,
   thereby inducing transcription of the fluorinase gene and the phosphorylase gene,
      thereby inducing fluorination and phosphorylation of the substrate to yield a fluorinated and phosphorylated product.

In one embodiment, the method is a method for in vivo fluorination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28) such as FlB1 or DeoD, said phosphorylase being capable of catalysing the phosphorylation of a fluorinated substrate, said fluorinase gene and phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a fluoride salt,
   whereby transcription of the gene encoding an activator of transcription is induced,
   thereby inducing transcription of the fluorinase gene and the phosphorylase gene,
      thereby inducing fluorination and phosphorylation of the substrate to yield a fluorinated and phosphorylated product.

In some embodiments, the inducer is a fluoride salt.

In one embodiment, the cell comprises:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter, wherein the fluorinase gene encodes the fluorinase of Streptomyces sp. MA37 as set forth in SEQ ID NO: 1 (FlA1), or a functional variant thereof having at least 80% homology thereto, and wherein the first promoter is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter and wherein the transcription of the fluorinase gene can be induced by an inducer;
b) a second nucleic acid comprising a riboswitch,
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter and a gene encoding a phosphorylase (EC 2.4.2.28) such as FlB1 or DeoD, said phosphorylase being capable of catalysing the phosphorylation of a fluorinated, chlorinated and/or brominated substrate such as a fluorinated, chlorinated and/or brominated adenosine, said phosphorylase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
   wherein the cell is capable of expressing the activator of transcription and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as FlB1 or DeoD, said phosphorylase being capable of catalysing the phosphorylation of a fluorinated, chlorinated and/or brominated adenosine, said fluorinase gene and phosphorylase gene being under control of the first promoter;
b) a second nucleic acid comprising a riboswitch;
   wherein the cell is capable of expressing the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a phosphorylase (EC 2.4.2.28) such as FlB1 or DeoD, said phosphorylase being capable of catalysing the phosphorylation of a fluorinated, chlorinated and/or brominated adenosine, said fluorinase gene and phosphorylase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
   wherein the cell is capable of expressing the activator of transcription, the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said nucleosidase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
   wherein the cell is capable of expressing the activator of transcription and the nucleosidase at least in the presence of inducer. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said nucleosidase, said fluorinase gene and nucleosidase gene being under control of the first promoter;
b) a second nucleic acid comprising a riboswitch;
   wherein the cell is capable of expressing the fluorinase and the nucleosidase at least in the presence of inducer. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, the conversion of 5′-chloro-5′-deoxyadenosine to 5′-chlorodeoxyribose and/or the conversion of 5′-bromo-5′-deoxyadenosine to 5′-bromodeoxyribose, said nucleosidase, said fluorinase gene and nucleosidase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
   wherein the cell is capable of expressing the activator of transcription, the fluorinase and the nucleosidase at least in the presence of inducer. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In another embodiment, the method is a method for in vivo chlorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—Cl bond, said fluorinase gene being under the control of the first promoter;
   b) a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a chloride salt,
   whereby transcription of the gene encoding an activator of transcription is induced,
   thereby inducing transcription of the fluorinase gene,
      thereby inducing chlorination of the substrate to yield a chlorinated product.

In one embodiment, the method is a method for in vivo chlorination of a substrate, comprising the steps of:
i) propagating a cell in a medium, said cell comprising:
   a) a first nucleic acid comprising, a first promoter and a fluorinase gene encoding a fluorinase (EC 2.5.1.63) capable of catalysing the formation of a C—Cl bond, said fluorinase gene being under the control of the first promoter and wherein the transcription of the fluorinase gene can be induced by an inducer;
   b) a second nucleic acid comprising a riboswitch,
i) adding the inducer to the medium and incubating the cell in the presence of inducer and a chloride salt,
   whereby transcription of the fluorinase gene is induced,
   thereby inducing chlorination of the substrate to yield a chlorinated product.

In one embodiment, the method is a method for in vivo phosphorylation of a chlorinated substrate, comprising the steps of:
i) propagating a cell in a medium, said cell comprising:
   a) a first nucleic acid comprising a first promoter and a gene encoding a phosphorylase (EC 2.4.2.28) such as FlB1 or DeoD, said phosphorylase being capable of catalysing the phosphorylation of the chlorinated substrate, said phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter
ii) adding the inducer to the medium and incubating the cell in the presence of inducer,
   whereby transcription of the gene encoding an activator of transcription is induced,
   thereby inducing transcription of the phosphorylase gene,
      thereby inducing phosphorylation of the chlorinated substrate to yield a chlorinated and phosphorylated product.

In one embodiment, the method is a method for in vivo chlorination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—Cl bond, and a gene encoding a phosphorylase (EC 2.4.2.28) such as FlB1 or DeoD, said phosphorylase being capable of catalysing the phosphorylation of a fluorinated substrate, said fluorinase gene and phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
i) adding the inducer to the medium and incubating the cell in the presence of inducer and a chloride salt,
   whereby transcription of the gene encoding an activator of transcription is induced,
   thereby inducing transcription of the fluorinase gene and the phosphorylase gene,
      thereby inducing chlorination and phosphorylation of the chlorinated substrate to yield a chlorinated and phosphorylated product.

In one embodiment, the method is a method for in vivo chlorination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—Cl bond, and a gene encoding a phosphorylase (EC 2.4.2.28) such as FlB1 or DeoD, said phosphorylase being capable of catalysing the phosphorylation of a fluorinated substrate, said fluorinase gene and phosphorylase gene being under control of the first promoter;
   b) a second nucleic acid comprising a riboswitch;
ii) adding the inducer to the medium and incubating the cell in the presence of inducer and a chloride salt,
   thereby inducing transcription of the fluorinase gene and the phosphorylase gene,
      thereby inducing chlorination and phosphorylation of the chlorinated substrate to yield a chlorinated and phosphorylated product.

In some embodiments, the inducer is a chloride salt. In other embodiments, the inducer is a fluoride salt.

In another embodiment, the method is a method for in vivo bromination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—Br bond, said fluorinase gene being under the control of the first promoter;
   b) a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a bromide salt,
   whereby transcription of the gene encoding an activator of transcription is induced,
   thereby inducing transcription of the fluorinase gene,
      thereby inducing bromination of the substrate to yield a brominated product.

In one embodiment, the method is a method for in vivo bromination of a substrate, comprising the steps of:
i) propagating a cell in a medium, said cell comprising:
   a) a first nucleic acid comprising first promoter and a fluorinase gene encoding a fluorinase (EC 2.5.1.63) capable of catalysing the formation of a C—Br bond, said fluorinase gene being under the control of the first promoter and wherein the transcription of the fluorinase gene can be induced by an inducer;
   b) a second nucleic acid comprising a riboswitch,
ii) adding the inducer to the medium and incubating the cell in the presence of inducer and a bromide salt,
   whereby transcription of the fluorinase gene is induced,
   thereby inducing bromination of the substrate to yield a brominated product.

In one embodiment, the method is a method for in vivo phosphorylation of a brominated substrate, comprising the steps of:
i) propagating a cell in a medium, said cell comprising:
   a) a first nucleic acid comprising a first promoter and a gene encoding a phosphorylase (EC 2.4.2.28) such as FlB1 or DeoD, said phosphorylase being capable of catalysing the phosphorylation of the brominated substrate, said phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter
ii) adding the inducer to the medium and incubating the cell in the presence of inducer,
   whereby transcription of the gene encoding an activator of transcription is induced,
   thereby inducing transcription of the phosphorylase gene,
      thereby inducing phosphorylation of the brominated substrate to yield a brominated and phosphorylated product.

In one embodiment, the method is a method for in vivo bromination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—Br bond, and and a gene encoding a phosphorylase (EC 2.4.2.28) such as FlB1 or DeoD, said phosphorylase being capable of catalysing the phosphorylation of a brominated substrate, said fluorinase gene and phosphorylase gene being under control of the first promoter;
   b) a second nucleic acid comprising a riboswitch;
ii) adding the inducer to the medium and incubating the cell in the presence of inducer and a bromide salt,
   thereby inducing transcription of the fluorinase gene and the phosphorylase gene,
      thereby inducing bromination and phosphorylation of the brominated substrate to yield a brominated and phosphorylated product.

In one embodiment, the method is a method for in vivo bromination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—Br bond, and a gene encoding a phosphorylase (EC 2.4.2.28) such as FlB1 or DeoD, said phosphorylase being capable of catalysing the phosphorylation of a brominated substrate, said fluorinase gene and phosphorylase gene being under control of the first promoter;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a bromide salt,
   whereby transcription of the gene encoding an activator of transcription is induced,
   thereby inducing transcription of the fluorinase gene and the phosphorylase gene,
      thereby inducing bromination and phosphorylation of the brominated substrate to yield a brominated and phosphorylated product.

In some embodiments, the inducer is a bromide salt. In other embodiments, the inducer is a fluoride salt.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the fluorinase gene encoding a fluorinase, the second nucleic acid, the riboswitch, the second promoter and the gene encoding an activator of transcription, may be as described herein.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the fluorinase gene encoding a fluorinase, and the riboswitch, may be as described herein.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the gene encoding a phosphorylase, the second nucleic acid, the riboswitch, the second promoter and the gene encoding an activator of transcription, may be as described herein.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the fluorinase gene encoding a fluorinase, the gene encoding a phosphorylase, the second nucleic acid, the riboswitch, the second promoter and the gene encoding an activator of transcription, may be as described herein.

The substrate and co-substrate may be as described herein.

The method may be performed by using an expression system suitable for expression of the first nucleic acid and of the second nucleic acid in a cell, as described herein. The method may be performed by using an expression system suitable for expression of the first nucleic acid in a cell, as described herein.

The method may be performed by using an expression system suitable for expression of the first nucleic acid and of the second nucleic acid in a cell, as described herein.

The method may be performed by using an expression system suitable for expression of the first nucleic acid and of the second nucleic acid in a cell, as described herein.

The type of medium used for propagating the cell will depend on the type of cell used for the method. The medium used is as known in the art.

### Inducer

The in vivo fluorination, chlorination or bromination of a substrate as described herein may be achieved by adding an inducer, which activates transcription of the gene encoding the activator of transcription. This in turn induces transcription of the fluorinase gene, as explained in detail herein above. As a result, the fluorinase is expressed, and can fluorinate, chlorinate or brominate the substrate in the presence of the appropriate co-substrate. In some embodiments, the co-substrate and the inducer are identical.

The inducer may be provided in the form of salts. Preferred salts are soluble salts. In some embodiments, the reaction is a fluorination reaction and the inducer is a fluoride salt. Preferred fluoride salts are NaF or KF; less soluble salts such as CaF₂ may also be used. In other embodiments, the reaction is a chlorination reaction and the inducer is a chloride salt. Preferred chloride salts are NaCl or KCl; less soluble salts such as CaCl₂) may also be used. In other embodiments, the reaction is a bromination reaction and the inducer is a bromide salt. Preferred bromide salts are NaBr or KBr; less soluble salts such as CaBr₂ may also be used.

The inducer may be added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

The time of adding the inducer may be optimised as is known in the art, and may depend on the choice of cell and/or on the choice of the fluorinase gene. In some embodiments, the inducer is added at culture onset or in mid-exponential phase. It may occur that the presence of inducer will inhibit or slow cell growth, why in some embodiments it may be advantageous to propagate the cells until mid-exponential phase prior to adding the inducer.

### Co-Substrate

The choice of co-substrate will typically be dictated by the type of in vivo reaction which is to be performed. In embodiments where in vivo fluorination of a substrate is desired, the co-substrate is fluoride. In embodiments where in vivo chlorination of a substrate is desired, the co-substrate is chloride. In embodiments where in vivo bromination of a substrate is desired, the co-substrate is bromide.

The co-substrates may be provided in the form of salts. Preferred salts are soluble salts. In some embodiments, the reaction is a fluorination reaction and the co-substrate is a fluoride salt. Preferred fluoride salts are NaF or KF; less soluble salts such as CaF₂ may also be used. In other embodiments, the reaction is a chlorination reaction and the co-substrate is a chloride salt. Preferred chloride salts are NaCl or KCl; less soluble salts such as CaCl₂) may also be used. In other embodiments, the reaction is a bromination reaction and the co-substrate is a bromide salt. Preferred bromide salts are NaBr or KBr; less soluble salts such as CaBr₂ may also be used.

The co-substrate may be added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

The time of adding the co-substrate may be optimised as is known in the art, and may depend on the choice of cell and/or on the choice of the fluorinase gene. In some embodiments, the co-substrate is added at culture onset or in mid-exponential phase. It may occur that the presence of co-substrate will inhibit or slow cell growth, why in some embodiments it may be advantageous to propagate the cells until mid-exponential phase prior to adding the co-substrate. In other embodiments, the co-substrate is present in the medium used for propagation from the beginning of the culture.

In some embodiments, the inducer is the co-substrate.

### Duration of Incubation

Once the inducer and/or co-substrate has been added to the medium, the cell is incubated for a duration such that fluorination, chlorination or bromination of the substrate is achieved to obtain the desired fluorinated, chlorinated or brominated product, respectively.

The duration of the incubation may depend on the choice of cell and of the type of reaction. In some embodiments, the cell is incubated in the presence of inducer for at least 4 hours, such as at least 8 hours, such as at least 12 hours, such as at least 16 hours, such as at least 20 hours, such as at least 24 hours, such as at least 36 hours, such as at least 48 hours, such as at least 72 hours, or more. Such incubation times, which are relatively short, are particularly relevant for embodiments where the cell is a bacterial cell, such as a P. putida cell, or a eukaryotic cell such as a yeast cell. In embodiments where the cell is a eukaryotic cell such as an insect cell, a mammalian cell or a plant cell, longer incubation times may be required. The skilled person knows how to determine which incubation times are suitable.

### Product Recovery

In some embodiments, the method further comprises a step of recovering the product, i.e. the fluorinated, chlorinated or brominated product.

The product will typically be present intracellularly and can be recovered as is known in the art, for example by solvent extraction from the cells (e.g. with an ethanol solution or a methanol solution). Preparative liquid chromatography can also be used to recover the product.

### Products and Compositions

Also provided herein is a fluorinated, a chlorinated or a brominated product obtainable by the methods described herein, and compositions comprising a fluorinated, a chlorinated or a brominated product obtainable by the methods described herein.

### Methods for Manufacturing a Fluorinated, a Chlorinated or a Brominated Compound of Interest

Also provided herein is a method for manufacturing a fluorinated, a chlorinated or a brominated compound of interest, said method comprising the steps of:
i) providing a fluorinated, a chlorinated or a brominated product by the methods described herein; and
ii) optionally converting said fluorinated, chlorinated or brominated product to the fluorinated, chlorinated or brominated compound of interest.

In some embodiments, the fluorinated, chlorinated or brominated product obtained by the methods described herein can be used directly, i.e. without further modification.

In other embodiments, however, the fluorinated, chlorinated or brominated compound is further converted into a fluorinated, chlorinated or brominated compound of interest. Indeed, the fluorinated, chlorinated or brominated product obtained by the present method may be used as a drug precursor. The methods of manufacturing a fluorinated, a chlorinated or a brominated compound of interest may thus comprise a step of converting the product obtained by the present methods into a compound of interest, such as a drug or a therapeutic compound. The compound of interest may be a fluorinated nucleoside, a deoxy-fluoronucleoside, a chlorinated nucleoside, a deoxy-chloronucleoside, a brominated nucleoside, a fluoro-ribose phosphate or a deoxy-bromonucleoside.

Also provided herein is the use of a fluorinated, a chlorinated or a brominated product obtainable by the present methods or of a composition comprising such product as a medicament.

### EXAMPLES

### Example 1—Materials and Methods

### Bacterial Strains and Growth Conditions

P. putida KT2440 was grown at 30° C. and routinely cultured in LB medium or on LB agar plates according to standard protocols unless otherwise stated. Modified M9 minimal medium containing 5 g L⁻¹ of glucose as carbon source was used for all the expression and production assays. E. coli DH5a was used for cloning and plasmid maintenance and was grown in LB at 37° C.

Antibiotics and other supplements were used at the following concentrations: chloramphenicol (Cm) 30 μg·mL⁻¹, kanamycin (Km) 50 μg·mL⁻¹, gentamicin (Gm) 10 μg·mL⁻¹, and streptomycin (St) 100 μg·mL⁻¹. 300 mM sucrose was used for preparing electrocompetent P. putida cells. Overnight pre-cultures were grown in modified M9 supplemented with 0.02% yeast extract. 3-methylbenzoate (3-MB) was used for induction of XylS/Pm promoter. Sodium fluoride (NaF) was purchased from Sigma-Aldrich (201154) and used as inducer, co-substrate and stressor.

Plasmids and strains construction pFB-PmflA1 was constructed by cloning the synthetic codon optimized gene flA1 from Streptomyces sp. MA37 into pPS23 vector, containing the expression system XylS/Pm, using the restriction sites AvrII and EcoRI. The expression vector pFB-PT7flA1 was constructed by cloning the gene flA1 into vector pSEVA231-T7pr, containing the expression system PT7, using the same restriction sites. For the construction of plasmid pFB-PT7flB1(NHis) the synthetic codon optimized gene flB1 from Streptomyces sp. MA37 (SEQ ID NO: 13) was amplified using a forward primer containing a 6× histidine sequence. The amplified gene was cloned into the plasmid pSEVA231-T7pr using AvrII and EcoRI restriction sites, as well. The construction of plasmid pFB-PT7flA1B1 was performed by USER cloning. For this purpose, plasmid pFB-PT7flA1 was amplified using the USER primers flA1_RSv1_U_rv and pSEVA_USER_flB, and gene flB1 was amplified. The amplified fragments were digested with DpnI and 100 ng of the corresponding DNA fragments were mixed with the USER enzyme in a final volume of 12 μl, and incubated at 37° C. for 25 minutes and 25° C. for 25 minutes. 5 μl of this mix was subsequently transformed by heat-shock into DH5α chemically competent cells and plated into LB agar plates with the corresponding antibiotics and incubated at 37° C. overnight. The correct constructions were checked by colony PCR and sequencing.

The four versions of the P. syringae riboswitch, FRSv1-4 (SEQ ID NO: 5, 6, 7 and 8, respectively), were synthesized by IDTDNA and cloned into plasmid pSEVA441, together with the msfGFP. The amplified fragments were digested with DpnI and 100 ng of the corresponding DNA fragments were mixed with the USER enzyme in a final volume of 12 μl, and incubated at 37° C. for 25 minutes and 25° C. for 25 minutes. 5 μl of this mix was subsequently transformed by heat-shock into DH5α chemically competent cells and plated into LB agar plates with the corresponding antibiotics and incubated at 37° C. overnight. The correct constructions were checked by colony PCR and sequencing. These clonings yielded plasmids pSEVA441-FRSv1, pSEVA441-FRSv2, pSEVA441-FRSv3, and pSEVA441-FRSv4.

Plasmid pFB-FRSflA1 was constructed by cloning the gene flA1, amplified with USER primers compatible with the riboswitch USER fragments, into plasmid pSEVA231, amplified with compatible USER primers as well. The fragments were treated with DpnI and 100 ng of the flA1, riboswitch FRSv1, and pSEVA231 fragments were mixed with the USER enzyme in a final volume of 12 ml, and incubated at 37° C. for 25 minutes and 25° C. for 25 minutes. 5 μl of this mix was subsequently transformed by heat-shock into DH5α chemically competent cells and plated into LB agar plates with the corresponding antibiotics and incubated at 37° C. overnight. The correct constructions were checked by colony PCR and sequencing.

The E. coli purine nucleotide phosphorylase, coded by the gene deoD (SEQ ID NO: 14), as well as the nucleosidase pfs (SEQ ID NO: 16), were amplified by PCR from the genome of the strain MG1655 using reverse primers containing the sequence to introduce a 6×His tag, in order to allow purification if needed. These fragments were respectively mixed with a PCR fragment amplified with USER primers of the pPS23 vector, containing the XylS/Pm expression system. The cloning was performed by USER cloning as described above.

The plasmid for integration of the T7 RNA polymerase under the regulation of the fluoride responsive riboswitch, pTn7::FRS-T7RNAP, was constructed by cloning the synthetic fragment FRSv1, containing the riboswitch and the original promoter from P. syringae, as well as the first amino acid of the gene eriC^{F}, to yield a translational fusion to the T7 RNA polymerase. The synthetic gene was amplified and cloned by USER cloning into the backbone pTn7::11_T7RNAP. Both DNA fragments were cloned by USER cloning as explained above.

For the deletion of crcB gene using CRISPR/Cas9, plasmid pSEVA231-CRISPRcrcB was constructed. Primers were phosphorylated by incubating 1 μl both primers at 100 μM with the enzyme T4 PNK and annealed simultaneously by incubating 30 minutes at 37° C., 4 minutes at 95° C., and decreasing the temperature 5° C. per minute until 25° C. were reached. The annealed primers were then ligated with plasmid pSEVA231-CRISPR, previously digested with the enzyme BsaI, using a T4 ligase enzyme. The ligation was then transformed by heat-shock into DH5α chemically competent cells and plated into LB agar plates with the corresponding antibiotics and incubated at 37° C. overnight. The correct constructions were checked by colony PCR.

To delete the crcB gene, an overnight culture of P. putida KT2440 containing the plasmids pSEVA658::SSR and pSEVA421::Cas9-tracrRNA was diluted to a A₆₀₀ of 0.1, and grown until the A₆₀₀ reached 0.4-0.5. Cells were subsequently induced to express the SSR recombinase with 1 mM of 3-MB for 3 hours at 30° C. After the induction, electrocompetent cells of the P. putida KT2440 induced strains were propagated using 300 mM sucrose, and were electroporated using 100 ng of pSEVA231-CRISPRcrcB plasmid and 1 μl of 100 M of crcBdeI-Rec, a recombineering oligo containing 45 bp of the upstream and downstream region including the start and stop codons of the gene crcB for its deletion. Cells were recovered in LB at 30° C. for 2 hours, plated in LB with the antibiotics needed, and incubated overnight at 30° C. The successful recombineering was checked by colony PCR and sequencing.

The integration of the T7 RNA polymerase regulated by the fluoride responsive riboswitch was achieved by electroporating the plasmid pTn7::FRS-T7RNAP together with the helper plasmid pTNS2, into freshly prepared P. putida KT2440 and P. putida KT2440ΔcrcB electrocompetent cells. The integration of the mini Tn7 module with the polymerase into the genome was checked by colony PCR.

### Expression Assays

Overnight cultures of P. putida KT2440 with plasmids pSEVA441-FRSv1, pSEVA441-FRSv2, pSEVA441-FRSv3, pSEVA441-FRSv4, or pSEVA441 as autofluorescence control, were diluted 40 times in 10 ml of minimal medium. Cells were grown in 96-well microtiter plates (flat bottom, Greiner Bio-one) for five hours at 30° C. and 300 rpm, after which the cultures were induced using the following concentrations: 0, 1, 5 and 15 mM of NaF. Subsequently, kinetics of the growth and fluorescence, using wavelengths of excitation and emission of 485 nm and 528 nm, respectively, were performed using a SynergyMX (BioTek) microtiter plate reader for 20 hours. Measurements for kinetics were carried out every ten minutes and three independent biological replicates were carried out. The same procedure was carried out with P. putida KT2440::FRS-T7RNAP and P. putida KT2440ΔcrcB::FRS-T7RNAP strains containing the plasmid pSEVA231-T7pr::msfGFP as well as with the plasmid pSEVA231-T7pr, used as an autofluorescence control. In this case 0, 1, and 15 mM of NaF was used for induction.

### In Vivo Production Assays

In vivo biofluorination in P. putida KT2440 harboring the plasmids pFB-PmflA1 was performed by diluting overnight cultures to an A₆₀₀ of 0.1 in fresh minimal medium, containing NaF 10 mM, in 50 ml in 250-ml shake flasks. Cells were grown for five to six hours at 30° C. with shaking of 200 rpm, when they reached an A₆₀₀ between 0.4-0.6, after which they were induced with 3-MB 1 mM. After 24 hours of incubation, samples were retrieved for supernatant analysis. The same strain with empty vector pPS23 was used as negative control. Two independent biological replicates were carried out for each condition.

In vivo biofluorination in P. putida KT2440ΔcrcB harboring the plasmids pFB-PmflA1 was performed by diluting overnight cultures to an A₆₀₀ of 0.1 in fresh minimal medium in 50 ml in 250-ml shake flasks. Cells were grown for one two hours at 30° C. with shaking of 200 rpm, when they reached an A₆₀₀ 0.2, after which they were induced with 3-MB 1 mM. After 6 hours of incubation, when cultures reached an A₆₀₀ 0.6, 0.5 nM of NaF was added. After 24 hours and 48 hours of incubation, samples were retrieved for supernatant analysis. The same strain with empty vector pPS23 was used as negative control. Two independent biological replicates were carried out for each condition.

In vivo biofluorination in P. putida KT2440 harboring the plasmids pFB-PT7flA1, pFB-FRSflA1, or pFB-FRSflA1B1 was performed by diluting overnight cultures to an A₆₀₀ of 0.1 in fresh minimal medium in 50 ml in 250-ml shake flasks. Cells were grown for four to five hours at 30° C. with shaking of 200 rpm, when they reached an A₆₀₀ between 0.4-0.6 or adding NaF 15 mM. After 24 hours of incubation, samples were retrieved for supernatant analysis and metabolite extractions. The same strain with empty vector pSEVA231-T7Pr was used as negative control. Two independent biological replicates were carried out for each condition.

For sample processing, cells were centrifuged at 5000 rpm for 10 minutes, and the supernatants and pellets were separated. For supernatant analysis, a second centrifugation was performed, and 1 ml of supernatant was used for the analysis in a LC-MS system. For the metabolite extraction from the pellets, cells were washed with a phosphate buffer, and incubated with 0.5 ml of extraction solution (ethanol 60% (v/v), ammonium acetate 10 mM, pH 7.2) at 78° C. for 1 minute. Cells were subsequently centrifuged at maximum speed for 1 minutes and the supernatant was placed in a new Eppendorf tube. This extraction was repeated three times for each sample and then evaporated in a Speed-vac for at least 6 hours. Pellets were eluted in 100 μl of water and analysed in a LC-MS system.

### In Vitro Production Assays

In vitro biofluorination was performed with the strain P. putida KT2440 harboring the plasmids pFB-PmflA1, pFB-PT7flB1(NHis), and pFB-PT7flA1, and pPS23 or pSEVA231-PT7pr as negative controls, respectively. Overnight cultures of the strains were diluted to an OD₆₀₀ of 0.1 in fresh minimal medium, in 250-ml shake flasks. Cells were grown for six hours at 30° C. with shaking of 200 rpm, when they reached an OD₆₀₀ between 0.4-0.6, after which they were induced with 3-MB 1 mM for the XylS/Pm system or 15 mM of NaF for the FRS-PT7. After 20 hours of incubation, all the volume of culture was centrifuged at 5000 rpm for 10 minutes at 4° C., and the supernatant was discarded. Cellular pellets were washed with Tris-HCl 50 mM pH7.8 and resuspended in a final volume of 3 ml. Cells were disrupted using glass beads in a bead beater, using a program of 6000 rpm for 20 seconds. The mix was centrifuged at 17000 g for 2 minutes at 4° C., and the supernatant was transferred to a new tube. Protein concentrations were determined using a Bradford assay, yielding a concentration of at least 1 mg/ml.

For the fluorinase activity assays, 1 ml of the protein fraction was then mixed with 0.2 mM of SAM and 5 mM of NaF, in Tris-HCl 50 mM pH7.8. This reaction was incubated for 20 hours at 30° C., and then inactivated by boiling the samples at 95° C. for 5 minutes and centrifuging at 17000 g for 10 minutes. The supernatant was taken and analysed by LC-MS.

Similarly, for phosphorylase activity assays, 1 ml of the protein fraction from cultures expressing the phosphorylase was then mixed with 0.2 mM of 5′-FDA, in Tris-HCl 50 mM pH7.8. 0.5 ml of the initial volume was inactivated by boiling the samples at 95° C. for 5 minutes and centrifuging at 17000 g for 10 minutes, yielding the initial concentration of 5′-FDA in the reaction (0 hours). After 20 hours of incubation at 30° C., the rest of the reaction was inactivated as described before and the supernatant was taken and analyzed by HPLC.

### Toxicity Assays

Overnight cultures of P. putida KT2440 and P. putida KT2440ΔcrcB were diluted 20 times in 10 mL of modified M9 minimal medium containing different concentrations of NaF. Cells were grown in 96-well microtiter plates (flat bottom, Greiner Bio-one) at 30° C. and 200 rpm in an ELx808 microtiter plate reader (BioTek). Cells exposed to NaF at various concentrations were incubated in a microtiter plate reader for 24 hours, and growth was followed by measuring OD₆₃₀ every 30 minutes. The concentrations used for each strain were 0, 25, 50, 75, 125, 200 and 250 mM for P. putida KT2440, and 0, 0.1, 0.5, 1 and 5 mM for P. putida KT2440ΔcrcB.

Growth arrest by NaF in P. putida KT2440ΔcrcB was performed by diluting 20 times the overnight cultures in 10 mL of modified M9 minimal medium. Cells were grown in 96-well microtiter plates (flat bottom, Greiner Bio-one) at 30° C. and 200 rpm in an ELx808 microtiter plate reader (BioTek). After six hours of incubation, the cultures had reached an OD₆₃₀ of 0.5, and the NaF was added at different concentrations. Cells exposed to the stressor were incubated in a microtiter plate reader for 24 hours, and growth was followed by measuring OD₆₃₀ every 30 minutes. The concentrations of NaF used were 0, 0.05, 0.1, 0.5, 0.75, 1, 2 and 5 mM.

### LC-MS Analysis

Detection of 5′-FDA were carried out by LC-MS measurements on a Dionex UltiMate 3000 UHPLC (Fisher Scientific, San Jose, CA) connected to an Orbitrap Fusion Mass Spectrometer (Thermo Fisher Scientific, San Jose, CA). The system used a Cortecs T3, 2.1×150 mm, 1.6 μm column kept at 30° C. The flow rate was 0.3 mL/min with a mobile phase of 100% formic acid (0.1%) for 4.2 min followed by a linear gradient of to 25% formic acid (0.1%)/75% acetonitrile (0.1% formic acid) over 1.3 min. This gradient was held for 1 min after which it was changed immediately to 100% formic acid (0.1%) and 0% acetonitrile (0.1% formic acid) and held for 6.5 min. The sample was passed on to the MS equipped with a heated electrospray ionization source (HESI) in positive-ion mode with nitrogen as nebulizer gas (40 a.u.). The cone and probe temperature were 317° C. and 335° C., respectively. Probe gas flow was 12 a.u. and spray voltage was 3500 V. Scan range was 50 to 400 Da and time between scans was 100 ms. All reagents used was of analytical grade. The detection of 5′-FDA was tested against an in-house synthesized standard.

Example 2—Fluorinase selection for its expression in P. putida KT2440 The canonical fluorination pathway, described for the first time in S. cattleya, utilizes SAM and NaF as substrates, which are converted by the fluorinase FlA into 5′-FDA. 5′-FDA is afterwards phosphorylated by a purine nucleotide phosphorylase (flB), and finally converted to fluoroacetaldehyde by an isomerase and an aldolase.

Fluoroacetaldehyde is subsequently transformed into fluoacetate or 4-fluorothreonine as the final products (Murphy 2003). The key step of the pathway, the formation of a C—F bond, is performed by the fluorinase in a very inefficient process due to the F chemistry. A comparison of several fluorinases has shown the inefficiency of such enzymes, which present turnovers lower than 0.3 min⁻¹. The fluorinase encoded by gene flA1 from Streptomyces sp. MA37 was selected for expression in P. putida for biofluorination. Due to the high G+C content of Streptomyces genes, a codon optimization was synthesized and, as a first approach to check the expression of the gene, was cloned under the regulation of the XylS/Pm system, a well-known high expression system in P. putida KT2440 (Calero 2016), yielding the plasmid pFB-PmflA1.

### Example 3—In Vitro and In Vivo Biofluorination in P. putida KT2440 Using a Xyls/Pm Expression System

The performance of the plasmid pFB-PmflA1 in P. putida KT2440 was tested in minimal medium supplemented with glucose and inducing the cells in exponential phase. After 24 hours of induction, we prepared a protein extract from the cultures and performed in vitro assays in which the extracts of P. putida KT2440 containing the fluorinase FlA1 were incubated with high concentrations of SAM and NaF. After 20 hours of incubation, a peak corresponding with the mass of 5′-FDA was found by LC-MS. The peak detected in these samples was then compared to a chemically synthesized 5′-FDA standard (data not shown). Protein extracts of the empty plasmid pPS23, containing only the XylS/Pm system, were incubated with SAM and NaF as well, but no 5′-FDA peak was found in those conditions. These results show that the fluorinase is being produced in P. putida KT2440 and that it is active enough for detection of the product of the enzyme.

To test the activity of the fluorinase in vivo, P. putida KT2440 cells harboring plasmid pFB-PmflA1 were grown in the presence of NaF 10 mM as substrate of the reaction. In this case, the same peak of the product of the fluorination reaction, 5′-FDA, that was found in the in vitro assays, was found in in the supernatants of P. putida KT2440 cultures. This peak presented the same mass as the custom chemically synthesized 5′-FDA standard peak as well. No trace of 5′-FDA was found in the supernatant of the cultures of P. putida KT2440 with plasmid pFB-PmflA1 that were not induced or when the empty plasmid pPS23 was used instead (data not shown). These results showed that the fluorinase FlA1 was being expressed and is was functional in vivo as well, although at much lower levels (data not shown). No difference in the growth was found between the strain harboring the pFB-PmflA1 when they were induced or not, although they were slower that the strains with the empty plasmid pPS23, showing a burden of the fluorinase plasmid even when this is not being expressed (data not shown).

### Example 4—Construction of a F⁻ Responsive Expression System Based on a Fluoride Responsive Riboswitch (FRS)

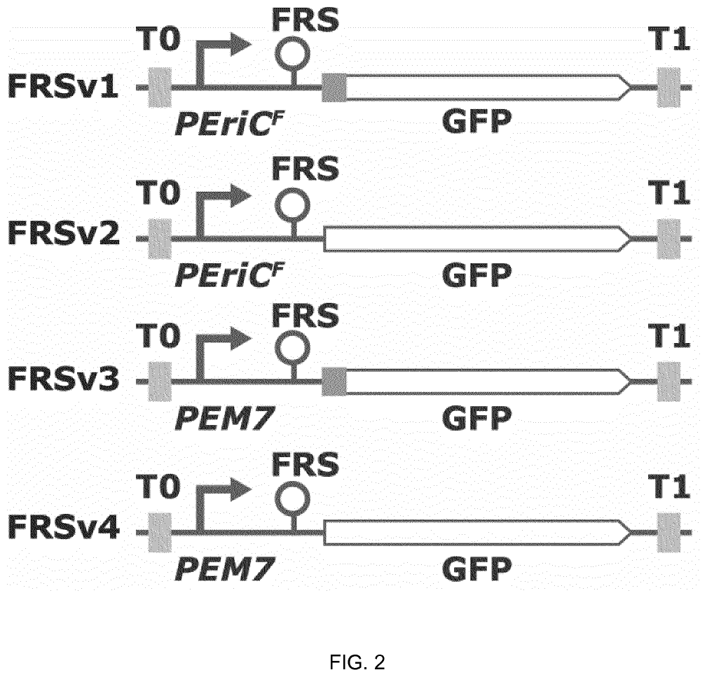

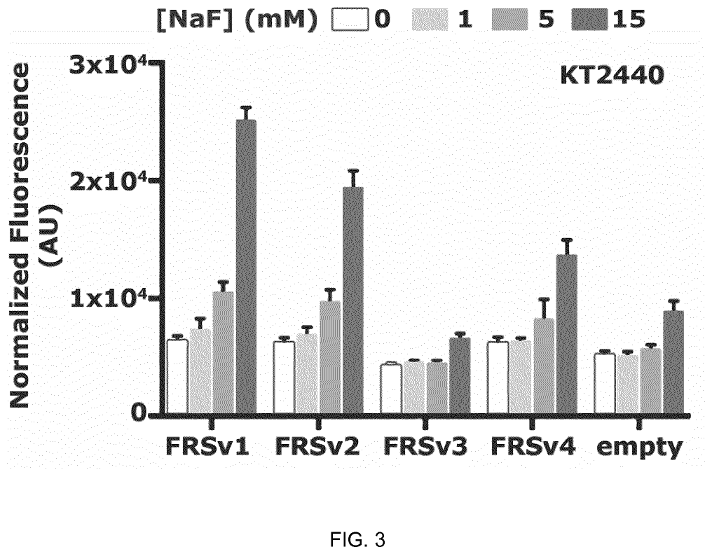

We explored the option of having an expression system that could respond to the addition of substrate, removing as many components of the system as possible. For this, we used the F⁻ responsive riboswitch (FRS) from P. syringae, which in its natural context drives the expression of the gene coding for the F⁻ transporter EriC^{F} in the presence of F⁻. To check the performance of the riboswitch in P. putida KT2440 we cloned four different versions of the riboswitch before the fluorescent reporter protein msfGFP. In the constructions FRSv1 and FRSv2 we kept the original promoter of gene eriC^{F}, whereas in FRSv3 and FRSv4 we changed the promoter to a constitutive heterologous promoter, PEM7. In the constructions FRSv1 and FRSv3 we conserved the first 8 amino acids of the original eriC^{F} gene as well, whereas FRSv2 and FRSv4 started with the coding sequence of the msfGFP (FIG. 2). The four constructions were tested in P. putida KT2440 after the addition of different concentrations of NaF, showing an increase of the fluorescence when higher concentrations of NaF were used (FIG. 3). This indicates that the riboswitch responds to NaF. Among the four constructions tested, the FRSv1 construction, with the original eriC^{F} promoter and the 8 first amino acids of the gene resulted to have the best response.

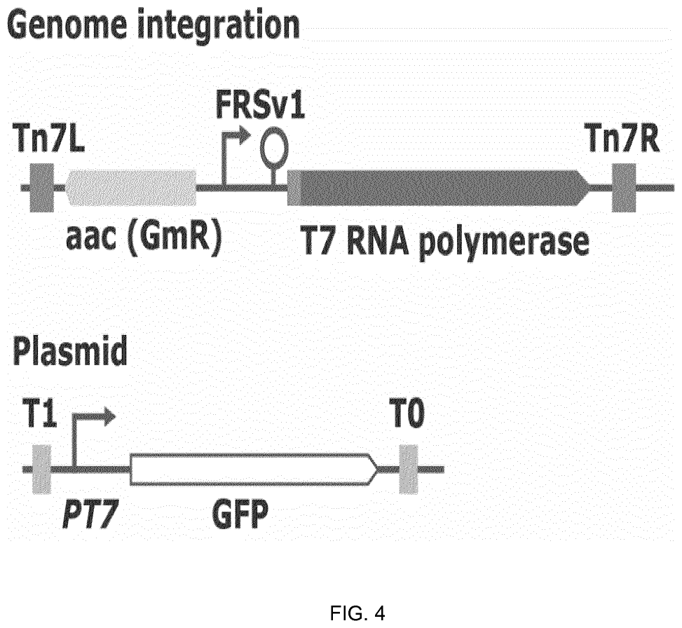

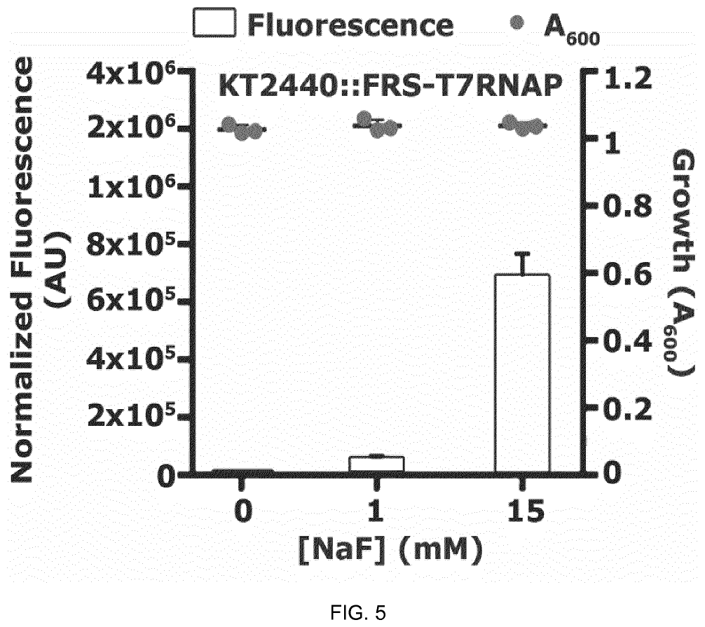

It has been shown previously that the genetic context of riboswitches affects critically the performance of such RNA regulatory devices (Kent 2018). Therefore, in order to create a system based on the FRS that is independent of the gene of interest, we cloned the promoter region of eriC^{F} gene that showed the best performance, FRSv1, controlling the expression of the T7 RNA polymerase. This construction allows the expression of the polymerase while keeping This FRS-PT7 system was therefore integrated it in single copy into the genome of P. putida KT2440, in the neutral region downstream glmS gene, using a mini-Tn7 delivery vector for this purpose. The resulting strain, P. putida KT2440::FRS-T7RNAP, was used as hosts for introduction of plasmids containing the PT7 promoter controlling any gene for their expression. To test its performance in P. putida, we cloned the gene coding for the fluorescent reporter protein msfGFP under the regulation of the PT7 promoter (FIG. 4). Cells harbouring this system were induced with different concentrations of NaF, which led to an increase of the cells fluorescence. The fluorescence when no inducer was added was very low, which indicated that the system is tight with low basal levels of expression. Moreover, the final growth of the cultures was not affected with the concentrations of NaF used (FIG. 5). This indicates that the system can achieve high levels of expression responding to the addition of NaF concentrations. However, the concentrations of NaF needed to activate the FRS-PT7 system were very high when compared to other expression systems available in P. putida. For example, the XylS/Pm system has been shown to need inducer concentrations as low as 0.5 mM of 3-MB to achieve full expression (Calero 2016).

### Example 5—Assessment of Intracellular NaF in P. putida KT2440 Using the FRS-PT7 System as a Biosensor and a Mutant in the NaF Transporter CrcB

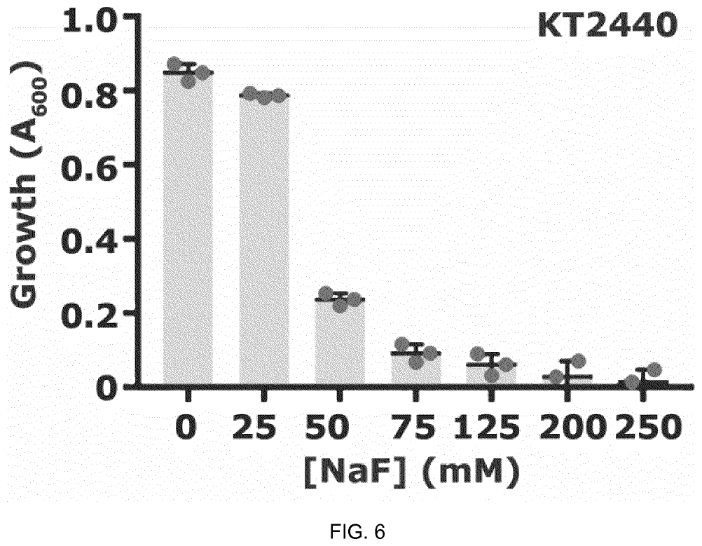

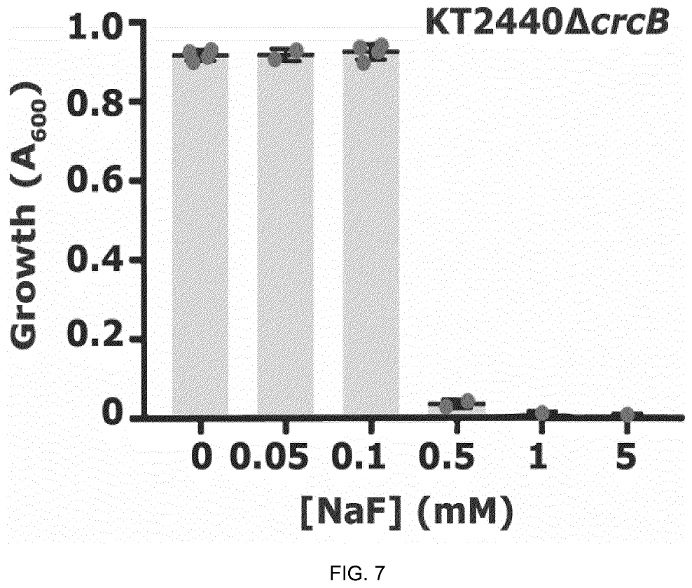

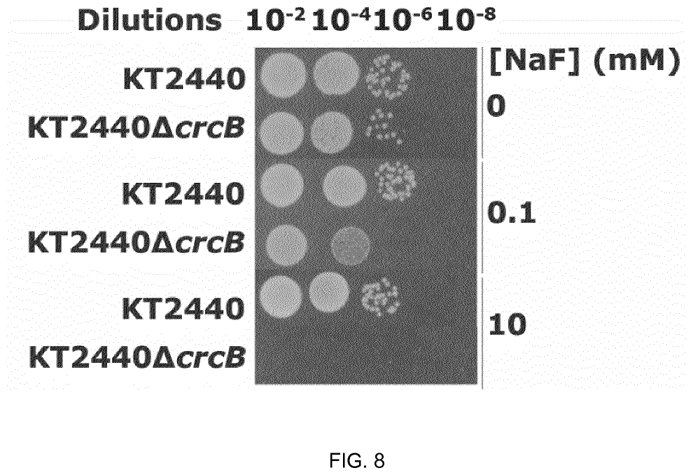

The intracellular availability of NaF in P. putida KT2440 cells was hypothesised to play an important role on the biofluorination. A gene annotated as a F⁻ transporter was found in the genome of P. putida KT2440. This gene, crcB, has also been shown to detoxify F⁻ in E. coli by extruding the ion out of the cells, keeping its intracellular concentration low (Baker 2012). In order to demonstrate the extruding action of CrcB in P. putida KT2440, an in phase scarless deletion of the gene crcB was constructed. The effect of the deletion of crcB on the growth in the presence of high concentrations of NaF in P. putida KT2440 was assessed by calculating the minimal inhibitory concentrations (MICs) of NaF needed to suppress the growth in both a wild-type and a ΔcrcB mutant. It was found that, whereas P. putida KT2440 was able to thrive in minimal medium supplemented with 75 mM of NaF (FIG. 6), the ΔcrcB mutant was not able to grow when 0.5 mM of NaF was added to the medium (FIG. 7). This effect was also seen in LB plates containing 0.5 and 1 mM of NaF (FIG. 8). These assays were performed as well in the presence of NaCl, and no such effect was seen (data not shown), showing that CrcB is a F-transporter that plays a crucial role in the survival of P. putida KT2440 in high concentrations of F⁻.

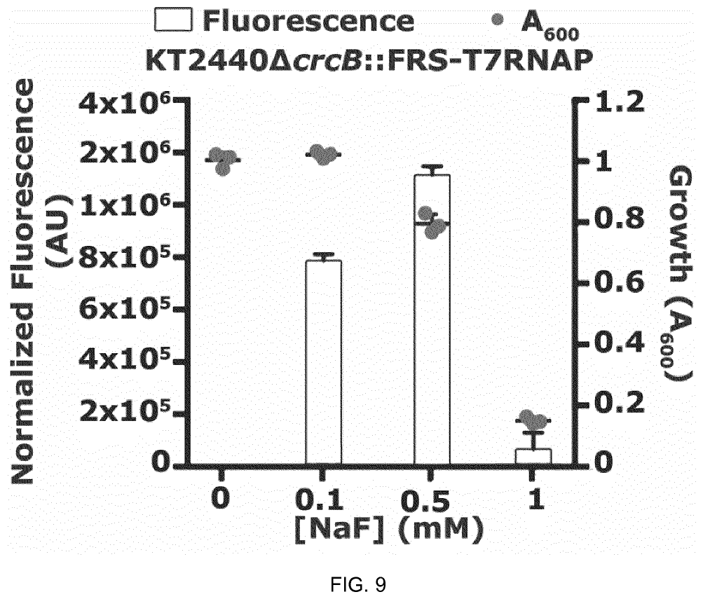

The transporter CrcB in P. putida KT2440 seems to keep low levels of intracellular concentrations of F⁻, as shown above. This could have an effect on the high concentrations of NaF needed to activate the FRS-T7. To test this hypothesis, we used the FRS-PT7 system as a biosensor for the intracellular concentration of NaF. Therefore, we constructed the strain P. putida KT2440ΔcrcB::FRS-T7RNAP, and introduced the PT7-msfgfp vector, to test the induction levels and concentrations of NaF needed. The concentrations of inducer used for this purpose were lower than in the P. putida KT2440 background, due to the toxic effect that is produced by the internal accumulation of F⁻ in the cells when the transporter is not present. However, even though the concentrations were considerably lower, the expression levels achieved in this background were almost two times higher than in the wild-type background (FIG. 9). These results indicate that the actual intracellular concentration of F⁻ was close to 0.1 mM in P. putida KT2440 even when 15 mM of NaF was added externally. It also shows that although lower concentrations of F⁻ can be used in the P. putida KT2440ΔcrcB mutant, the actual intracellular concentration in this background is higher, so it could be a most suitable host for biofluorinations.

### Example 6—In Vivo Growth-Decoupled Biofluorination in P. putida KT2440ΔcrcB

To explore the possibility of having a growth-decoupled platform for biofluorination, we tested the effect of NaF in the growth when added in mid-exponential phase. In this context, cells were expected to tolerate higher concentrations than when the stressor was added from the beginning during lag phase. Effectively, in this case when 0.5 mM of NaF was added in mid-exponential phase, a decrease in the growth phase was found, although a not so low final growth was seen. That was not the case when 1 mM of NaF was added, a condition that stopped cell growth almost completely (data not shown). In addition to the harsh effect on growth that F⁻ has in cell growth when the homeostasis is disrupted, these results also show that by changing the addition of NaF, the growth arrest can be modulated during the production process.

We therefore tested the production of 5′-FDA in a P. putida KT2440ΔcrcB background using the pFB-Pmfla1 plasmid. In contrast with the biofluorination assays using the wild-type P. putida KT2440 when the NaF was present in the medium from the beginning, in the biofluorination assays using P. putida KT2440ΔcrcB the NaF was added when a A₆₀₀ of approximately 0.5 was reached. We also observed that the simultaneous addition of the inducer 3 MB and the substrate NaF affected negatively the growth as well as the production, and no biofluorination was detected (data not shown). However, when the inducer 3 MB was added early (A₆₀₀ of about 0.2) and then the NaF was added in mid exponential phase, an effect on the growth was seen but production of 5′-FDA was achieved after 24 hours (data not shown). After 48 hours of production, cells are able to grow up to A₆₀₀ of 2.5 (data not shown).

### Example 7—In Vivo Biofluorination Using the Substrate NaF as Inducer

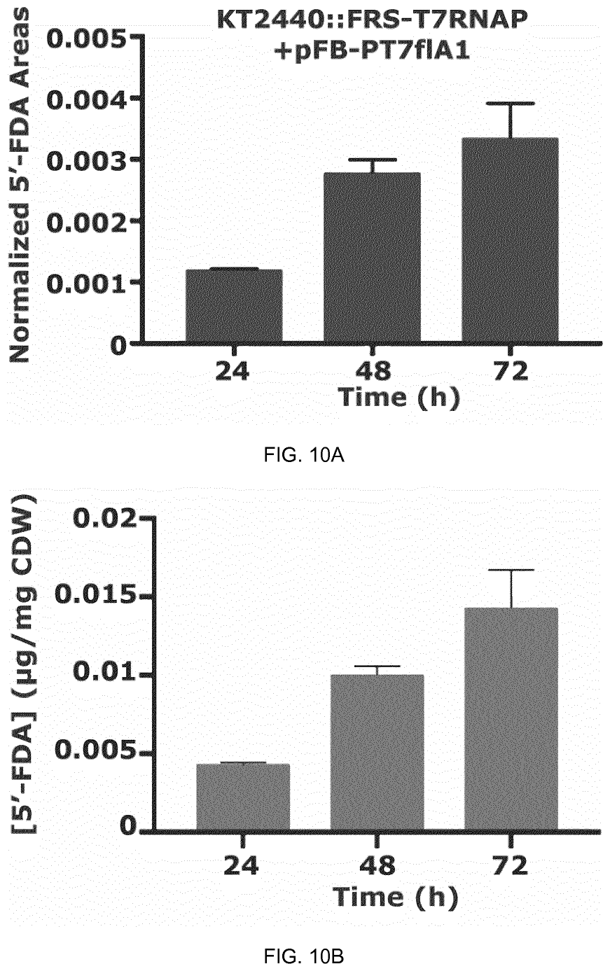

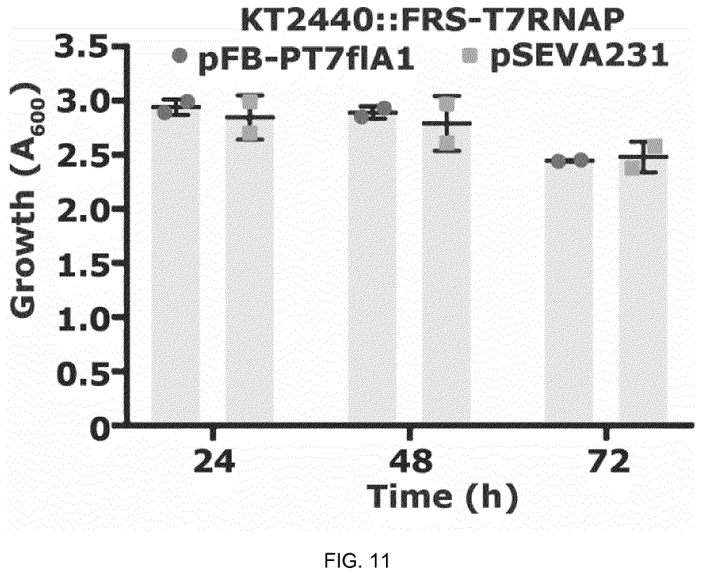

The FRS-T7 system was shown to work in P. putida KT2440 in presence of the inducer NaF. In order to give a further application to the system than a biosensor of the substrate NaF, we used it as an expression system using NaF, an inorganic salt, as the inducer. For this purpose, we cloned the fluorinase gene flA1 under the regulation of promoter PT7 and transformed it into the strains harbouring the FRS-T7 RNA polymerase system (FIG. 1). We tested the in vivo biofluorination inducing the strain P. putida KT2440::FRS-T7RNAP with the plasmid pFB-PT7flA1 with 15 mM of NaF. The supernatants of the cultures were measured in the LC-MS after 24 hours, but no production of 5′-FDA was found. However, when we extracted the metabolites of the cultures, the peak of 5′-FDA was detected. We extended the production times to 48 hours and 72 hours, in order to see when the 5′-FDA could be found in the supernatant, and after 72 hours the 5′-FDA was detected in the supernatant in the LC-MS. After 48 hours and 72 hours, increasing concentrations of 5′-FDA could be found in the metabolite extractions from the cellular pellets (FIG. 10). This indicates that the production of 5′-FDA is stable during at least 72 hours in in vivo assays in shake flasks. The absence of 5′-FDA in the supernatant before 72 hours also indicates that it is due to cell lysis, pointing to the fact that the production from the FRS-T7 system keep the cells in a more fit state than the XylS/Pm system. This is also supported by the fact that the final growth after 24 hours is higher in the P. putida KT2440::FRS-T7RNAP with the plasmid pFB-PT7flA1 than in the P. putida KT2440 with the plasmid pFB-PmflA1 (FIG. 11).

We tested the possibility of using the F⁻ responsive riboswitch as an expression system for the in vivo fluorination, which would additionally use the NaF as inducer. For this purpose, we constructed plasmid pFB-FRSflA1 (FIG. 12), cloning the flA1 gene under the regulation of the promoter region of eriC^{F} gene that showed the best performance, FRSv1. This vector was subsequently transformed into P. putida KT2440 and in vivo fluorination assays were performed using the resulting strain, inducing the expression of the fluorinase using 15 mM of NaF. After 20 and 48 hours, we extracted the metabolites from the cells and analysed the extractions in the LC-MS, detecting peaks of 5′-FDA of up to 0.006 μg per mg of cell dry weight (FIG. 13). This experiment thus shows that the system allows production of fluorinated compounds.

### Example 8—In Vivo Production of 5′-Fluorodeoxyribose Phosphate

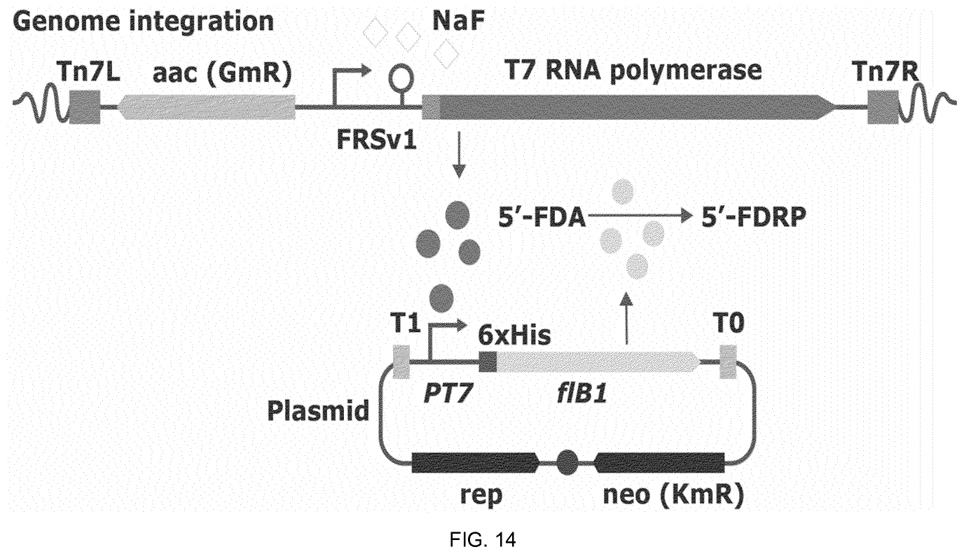

The organofluorine 5′-FDA can be further converted into the fluorinated sugar 5′-fluorodeoxyribose phosphate (5′-FDRP) by the action of a purine nucleotide phosphorylase (PNP) from S. cattleya, coded by gene flB. In order to assess the ability of P. putida KT2440 to express this gene, the codon optimized gene flB1 from Streptomyces sp. MA37 (SEQ ID NO: 13) was cloned under the regulation of the T7 promoter, to be used in the F⁻ inducible expression system based on the T7 RNA polymerase that we described before for the fluorinase expression (FIG. 14).

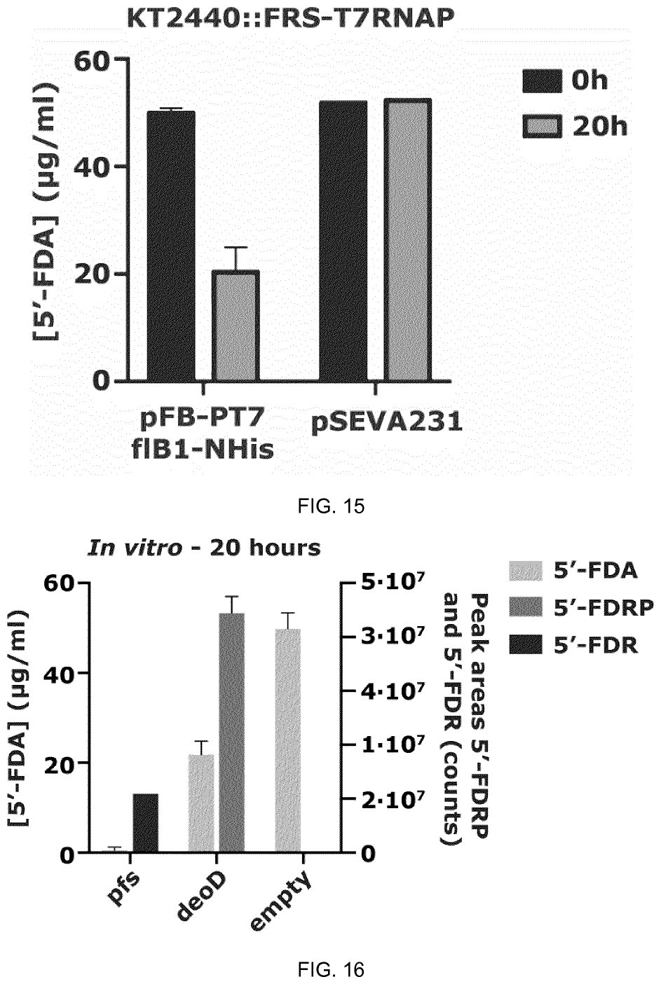

The expression of flB1 (SEQ ID NO: 11) in P. putida KT2440::FRS-T7RNAP was tested in minimal medium supplemented with glucose and inducing the cells in exponential phase. After 24 hours of induction, we prepared a protein extract from the cultures and performed in vitro assays in which the extracts of P. putida KT2440::FRS-T7RNAP containing the PNP FlB1 were incubated with high concentrations of 5′-FDA. After 20 hours of incubation, a decrease in the concentration of 5′-FDA compared to the initial concentration added to the reactions could be seen in the cell extracts from cultures with plasmid pFB-T7FlB1(NHis). No decrease in the 5′-FDA concentration could be detected when cell extracts of the empty plasmid pSEVA231 were used (FIG. 15). The appearance of a 5′-FDRP in these samples was also detected using LC-MS, supporting the fact that the PNP is active. This shows that the cells were able to phosphorylate 5′-FDA when expressing a phosphorylase.

We furthermore explored the possibility that 5′-FDA could be utilized for other enzymes different to the endogenous of the fluorination pathway from Streptomyces. For this purpose, we used another PNP and a nucleosidase of E. coli. The E. coli PNP, coded by the gene deoD (SEQ ID NO: 14), is able to perform the same reaction as FlB1, and produce 5′-FDRP, whereas the nucleosidase, coded by the gene pfs (SEQ ID NO: 16), removes the adenosine of 5′-FDA, yielding the sugar 5′-fluorodeoxyribose. These three enzymes share a similarity of 26% in their amino acidic sequence, and contain a common domain, the PNP_UDP_1 domain (PF0148), belonging to the Phosphorylase superfamily. To test the activity of these two enzymes, they were overexpressed in P. putida and the cell extracts were incubated with the substrate 5′-FDA. After 20 hours, the substrate of the reaction was reduced or depleted completely, and the products could be detected by LC-MS (FIG. 16), as compared to cell extracts with the empty plasmid, where no conversion was observed. This shows that cells expressing the E. coli PNP and cells expressing the Pfs nucleosidase are able to use 5′-FDA as a substrate to produce 5′-FDRP and 5′-fluorodeoxyribose, respectively.

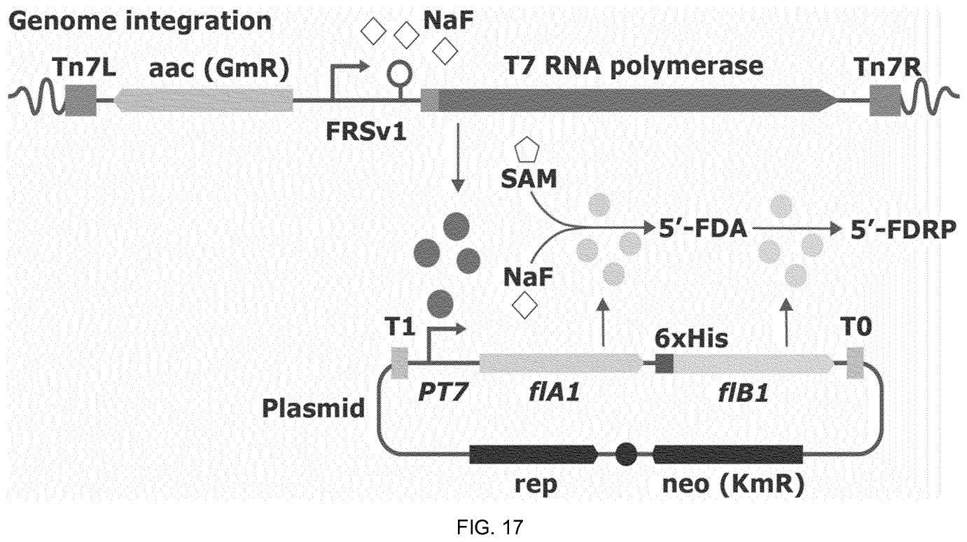

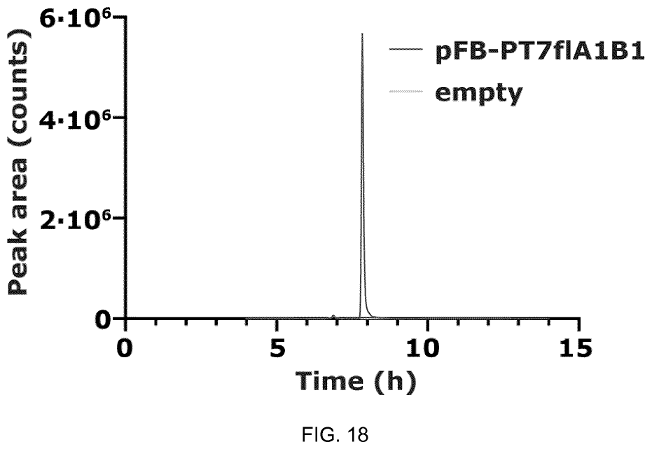

We finally tested the ability of the cells to synthesize in vivo 5′-FDRP using F⁻ as the inducer and substrate of the reaction. For this purpose, we constructed the plasmid pFB-PT7flA1B1, which contains genes flA1 and flB1, both under the regulation of the promoter PT7. This plasmid was transformed into the strain that contains the T7 RNA polymerase under the regulation of the riboswitch FRS, so the whole expression system responds to F⁻ (FIG. 17). We induced the KT2440::FRS-T7RNAP cultures with plasmid pFB-PT7flA1B1 with 15 mM of NaF, and incubated the cultures for 24 hours after the induction. After this time, 5′-FDRP could be detected using LC-MS in both the supernatant and the cell cytosol, whereas no peak could be seen in the cultures of P. putida KT2440::FRS-T7RNAP harbouring the empty plasmid (FIG. 18). This experiment shows that cells engineered to express a fluorinase and a phosphorylase can produce phosphorylated and fluorinated compounds such as 5′-FDRP.

### REFERENCES

Baker, Sudarsan, Weinberg, Roth, Stockbridge, Breaker (2012). Widespread Genetic Switches and Toxicity Resistance Proteins for Fluoride. Science. 335(6065): 233-235 Calero, Jensen, Nielsen (2016). Broad-Host-Range ProUSER Vectors Enable Fast Characterization of Inducible Promoters and Optimization of p-Coumaric Acid Production in Pseudomonas putida KT2440. ACS Synth Biol. 2016, 5, 741-753
Calero and Nikel (2018) Chasing bacterial chassis for metabolic engineering: a perspective review from classical to non-traditional microorganisms. Microb Biotechnol. 2018 Jun. 21.
Clayton Speed, Burkhart, Picking, Santangelo (2018). An Archaeal Fluoride-Responsive Riboswitch Provides an Inducible Expression System for Hyperthermophiles. Appl Environ Microbiol. 2018 Mar. 19; 84(7).
Danchin (2012) Scaling up synthetic biology: Do not forget the chassis. FEBS Letters 586 (2012) 2129-2137
Deng, O'Hagan, Schaffrath (2004) Fluorometabolite biosynthesis and the fluorinase from Streptomyces cattleya. Nat. Prod. Rep. 21, 773-784
Deng, Ma, Bandaranayaka, Qin, Mann, Kyeremeh, Yu, Shepherd, Naismith, O'Hagan (2014) Identification of Fluorinases from Streptomyces sp MA37, Norcardia brasiliensis, and Actinoplanes sp N902-109 by Genome Mining. ChemBioChem 2014, 15, 364-368
Eustaquio, O'Hagan, Moore (2010) Engineering Fluorometabolite Production: Fluorinase Expression in Salinispora tropica Yields Fluorosalinosporamide. J. Nat. Prod. 2010, 73, 378-382
Kent, Halliwell, Young, Swainston, Dixon (2018) Rationalizing Context-Dependent Performance of Dynamic RNA Regulatory Devices. ACS Synth Biol. 2018 Jul. 20; 7(7): 1660-1668
Liu, Evans, Zhang (2015) Applications and advances of metabolite biosensors for metabolic engineering. Metabolic Engineering 31(2015) 35-43
Ma, Li, Meng, Deng, Li, Zhang, Diao (2016) Biological fluorination from the sea: discovery of a SAM-dependent nucleophilic fluorinating enzyme from the marine-derived bacterium Streptomyces xinghaiensis NRRL B24674. RSC Adv., 6, 27047
Morra, Shankar, Robinson, Halliwell, Butler, Upton, Hay, Micklefield, Dixon (2015) Dual transcriptional-translational cascade permits cellular level tuneable expression control. Nucleic Acids Research, 2016, Vol. 44, No. 3
Murphy, Schaffrath, O'Hagan (2003). Fluorinated natural products: the biosynthesis of fluoroacetate and 4-fluorothreonine in Streptomyces cattleya. Chemosphere 52, 455-461.
O'Hagan, Schaffrath, Cobb, Hamilton, Murphy (2002) Biosynthesis of an organofluorine molecule. Nature vol. 416: 279-
O'Hagan (2008). Understanding organofluorine chemistry. An introduction to the C—F bond. Chem. Soc. Rev., 2008, 37, 308-319
Stockbridge, Lim, Otten, Williams, Shane, Weinberg, Miller (2012) Fluoride resistance and transport by riboswitch-controlled CLC antiporters. PNAS vol. 109 no. 38: 15289-15294
Sun, Yeo, Lim, Chew, Smith, Xue, Chan, Robinson, Robins, Zaho, Ang (2016). Directed Evolution of a Fluorinase for Improved Fluorination Efficiency with a Non-native Substrate. Angew Chem Int Ed Engl. 2016 Nov. 7; 55(46): 14277-14280 Thomsen, Vogensen, Buchardt, Burkart, Clausen (2013) Org. Biomol. Chem. 11:7606-7610.
Yeo, Chew, Smith, Chan, Sun, Zhao, Lim, Ang (2017) Probing the molecular determinants of fluorinase specificity. Chem. Commun., 2017, 53, 2559-2562
Zhang, Li, Jiang (2012). Fluorine is Fluorishing in Pharmaceuticals. J Biomol Res Ther, 1:2.
Zhou, Wang, Gu, Wang, Zhu, Aceña, Soloshonok, Izawa, Liu (2016) Next Generation of Fluorine-Containing Pharmaceuticals, Compounds Currently in Phase II-III Clinical Trials of Major Pharmaceutical Companies: New Structural Trends and Therapeutic Areas. Chem. Rev. 2016, 116, 422-518
Zhu, Robinson, McEwan, O'Hagan, Naismith (2007) The mechanism of the enzymatic fluorination in Streptomyces cattleya. J Am Chem Soc. 2007 Nov. 28; 129(47): 14597-14604

### Sequence Overview

| Sequence | | |
|---|---|---|
| ID NO: | | |
| SEQ ID | FIA1 from | MAANGSQRPIIAFMSDLGTTDDSVAQ |
| NO: 1 | Streptomyces | CKGLMHSICPGVTVVDVCHSMTPWDV |
| | sp. MA37, | EEGARYIVDLPRFFPEGTVFATTTYP |
| | protein | ATGTTTRSVAVRIRQAAKGGARGQWA |
| | sequence | GSGDGFERADGSYIYIAPNNGLLTTV |
| | | LEEHGYIEAYEVTSTKVIPANPEPTF |
| | | YSREMVAIPSAHLAAGFPLAEVGRRL |
| | | DDSEIVRFHRPAVEISGEALSGVVTA |
| | | IDHPFGNIWTNIHRTDLEKAGIGQGK |
| | | HLKIILDDVLPFEAPLTPTFADAGAI |
| | | GNIAFYLNSRGYLSLARNAASLAYPY |
| | | NLKAGLKVRVEAR |
| SEQ ID | fIA1 from | TGGTGGCTGCAAACGGCTCTCAGCGC |
| NO: 2 | Streptomyces | CCCATCATCGCCTTCATGAGCGACCT |
| | sp. MA37, | CGGCACCACGGACGACTCGGTGGCCC |
| | native | AGTGCAAGGGCCTCATGCACAGCATC |
| | | TGCCCCGGCGTCACGGTGGTGGACGT |
| | | CTGCCACTCCATGACGCCGTGGGACG |
| | | TCGAGGAGGGTGCGCGCTACATCGTC |
| | | GACCTCCCCCGCTTCTTCCCCGAGGG |
| | | CACGGTGTTCGCCACCACCACCTATC |
| | | CCGCGACCGGCACCACGACCCGCTCC |
| | | GTGGCCGTGCGGATCCGGCAGGCGGC |
| | | CAAGGGCGGCGCCCGCGGCCAGTGGG |
| | | CCGGCTCCGGCGACGGCTTCGAGCGG |
| | | GCCGACGGCTCGTACATCTACATCGC |
| | | GCCCAACAACGGCCTGCTGACCACCG |
| | | TGCTCGAGGAGCACGGATACATCGAG |
| | | GCCTACGAGGTCACGTCCACCAAGGT |
| | | CATCCCCGCGAACCCCGAACCGACCT |
| | | TCTACAGCCGCGAGATGGTGGCCATC |
| | | CCCTCCGCCCACCTCGCCGCCGGCTT |
| | | CCCGCTGGCCGAGGTCGGCCGCCGGC |
| | | TCGACGACAGCGAGATCGTCCGCTTC |
| | | CACCGGCCCGCGGTCGAGATCTCCGG |
| | | GGAGGCGCTCTCCGGCGTCGTCACCG |
| | | CGATCGACCACCCCTTCGGCAACATC |
| | | TGGACCAACATCCACCGCACGGACCT |
| | | GGAGAAGGCCGGCATCGGCCAGGGGA |
| | | AGCACCTGAAGATCATCCTCGACGAC |
| | | GTCCTGCCGTTCGAGGCCCCGCTCAC |
| | | CCCGACCTTCGCGGACGCCGGGGCAA |
| | | TCGGCAACATCGCCTTCTACCTCAAC |
| | | AGCCGCGGCTACCTGTCCCTCGCCCG |
| | | CAACGCCGCCAGCCTCGCCTACCCCT |
| | | ACAACCTCAAGGCCGGTCTGAAGGTC |
| | | CGCGTGGAAGCGCGCTGA |
| SEQ ID | fIA1 from | ATGGCCGCCAACGGCAGCCAGCGCCC |
| NO: 3 | Streptomyces | GATCATCGCGTTCATGTCCGACCTGG |
| | sp. MA37, | GCACCACCGACGACAGCGTGGCGCAG |
| | codon | TGCAAGGGCCTGATGCACAGCATCTG |
| | optimized | CCCCGGTGTGACCGTGGTGGATGTGT |
| | | GCCACAGCATGACCCCGTGGGACGTC |
| | | GAGGAGGGCGCCCGTTACATCGTGGA |
| | | CCTGCCGCGTTTCTTCCCGGAGGGCA |
| | | CCGTCTTCGCCACCACCACCTATCCC |
| | | GCCACCGGCACCACCACCCGCAGCGT |
| | | GGCCGTGCGTATCCGCCAGGCCGCCA |
| | | AAGGCGGCGCCCGTGGCCAGTGGGCC |
| | | GGCAGCGGCGACGGCTTCGAACGTGC |
| | | CGACGGCAGCTACATCTACATCGCCC |
| | | CGAACAACGGCCTGCTGACCACCGTG |
| | | CTGGAGGAACACGGCTATATCGAGGC |
| | | CTACGAGGTGACCAGCACCAAGGTGA |
| | | TCCCGGCGAACCCGGAGCCGACCTTC |
| | | TACAGCCGCGAAATGGTGGCCATCCC |
| | | GTCCGCCCACCTGGCCGCCGGCTTCC |
| | | CGCTGGCCGAGGTGGGCCGTCGTCTG |
| | | GATGACAGCGAGATCGTCCGTTTCCA |
| | | CCGCCCCGCCGTCGAGATCTCCGGCG |
| | | AAGCCCTGAGCGGCGTGGTGACCGCC |
| | | ATCGACCACCCGTTCGGCAACATCTG |
| | | GACCAACATCCACCGTACCGACCTGG |
| | | AAAAGGCCGGCATCGGCCAGGGCAAA |
| | | CACCTGAAGATCATCCTGGACGACGT |
| | | GCTGCCGTTCGAAGCCCCGCTGACCC |
| | | CCACCTTCGCCGACGCCGGCGCCATC |
| | | GGCAACATCGCCTTCTACCTGAACAG |
| | | CCGCGGCTATCTGAGCCTGGCCCGCA |
| | | ACGCCGCCAGCCTGGCCTATCCGTAC |
| | | AACCTGAAAGCCGGCCTGAAGGTGCG |
| | | CGTGGAGGCCCGTTGA |
| SEQ ID | FRS: | TTTGGCCCTCTTTCGTAAGCGGTGAC |
| NO: 4 | Fluoride | TCGAAGACGCCTTCGCTTGCCTCGTA |
| | responsive | CTCCGAAACACGATTTTACATTTTGG |
| | riboswitch | ACAGACCTAGCTAAGATCGGCGCATT |
| | from | GGAGATGGCATTCCTCCATTAACAAA |
| | Pseudomonas | CCGCTGCGCCCGTAGCAGCTGATGAT |
| | syringae | GCCTACAGAAACCTGATCAAACCAGG |
| | | TCTGTAGGCGTTCGCGCTTAGAATCC |
| | | CTTCTTTGGTCAGGCCCACTTATTTT |
| | | TTGTGGCTGGCCAA |
| SEQ ID | FRSv1: | ATCCTCTTTTGGCCCTCTTTCGTAAG |
| NO: 5 | Pseudomonas | CGGTGACTCGAAGACGCCTTCGCTTG |
| | syringae FRS | CCTCGTACTCCGAAACACGATTTTAC |
| | and first | ATTTTGGACAGACCTAGCTAAGATCG |
| | amino acids | GCGCATTGGAGATGGCATTCCTCCAT |
| | of EriCF | TAACAAACCGCTGCGCCCGTAGCAGC |
| | | TGATGATGCCTACAGAAACCTGATCA |
| | | AACCAGGTCTGTAGGCGTTCGCGCTT |
| | | AGAATCCCTTCTTTGGTCAGGCCCAC |
| | | TTATTTTTTGTGGCTGGCCAAATGTC |
| | | TAAATTTCGACGACCT |
| SEQ ID | FRSv2: | ATCCTCTTTTGGCCCTCTTTCGTAAG |
| NO: 6 | Pseudomonas | CGGTGACTCGAAGACGCCTTCGCTTG |
| | syringae FRS | CCTCGTACTCCGAAACACGATTTTAC |
| | | ATTTTGGACAGACCTAGCTAAGATCG |
| | | GCGCATTGGAGATGGCATTCCTCCAT |
| | | TAACAAACCGCTGCGCCCGTAGCAGC |
| | | TGATGATGCCTACAGAAACCTGATCA |
| | | AACCAGGTCTGTAGGCGTTCGCGCTT |
| | | AGAATCCCTTCTTTGGTCAGGCCCAC |
| | | TTATTTTTTGTGGCTGGCCAA |
| SEQ ID | FRSv3: PEM7 | ATCCTCTTTGTTGACAATTAATCATC |
| NO: 7 | promoter, | GGCATAGTATATCGGCATAGTATAAT |
| | Pseudomonas | ACGACAAGGTGAGGAACTAAACCAGA |
| | syringae FRS | CCTAGCTAAGATCGGCGCATTGGAGA |
| | and first | TGGCATTCCTCCATTAACAAACCGCT |
| | codons of | GCGCCCGTAGCAGCTGATGATGCCTA |
| | EriC^{F} | CAGAAACCTGATCAAACCAGGTCTGT |
| | | AGGCGTTCGCGCTTAGAATCCCTTCT |
| | | TTGGTCAGGCCCACTTATTTTTTGTG |
| | | GCTGGCCAAATGTCTAAATTTCGACG |
| | | ACCT |
| SEQ ID | FRSv4: PEM7 | ATCCTCTTTGTTGACAATTAATCATC |
| NO: 8 | promoter, | GGCATAGTATATCGGCATAGTATAAT |
| | Pseudomonas | ACGACAAGGTGAGGAACTAAACCAGA |
| | syringae FRS | CCTAGCTAAGATCGGCGCATTGGAGA |
| | | TGGCATTCCTCCATTAACAAACCGCT |
| | | GCGCCCGTAGCAGCTGATGATGCCTA |
| | | CAGAAACCTGATCAAACCAGGTCTGT |
| | | AGGCGTTCGCGCTTAGAATCCCTTCT |
| | | TTGGTCAGGCCCACTTATTTTTTGTG |
| | | GCTGGCCAA |
| SEQ ID | crcB from | ATGATTGCACTCATCGCCGCGGTCAG |
| NO: 9 | Pseudomonas | CGCCGGTGGTATTGCTGGTACCTTGT |
| | putida | TGCGCTTTGCTACCACCAACTGGGTG |
| | (PP_4001) | GCGGCTCACTGGCCACGGCACTTCTA |
| | | TGCCGGTACGCTGGCGGTCAACCTGG |
| | | TTGGCTGCCTGCTGATCGGCTTGCTC |
| | | TATGGCTTGTTCTTGCACAAGCCGCT |
| | | GGCGCCGGTCGAATTGCGCGCCGGCC |
| | | TGATTGTCGGCTTTCTCGGTGGCCTG |
| | | ACGACCTTTTCCTCCTTCTCGCTCGA |
| | | TACCGTGCGCCTGATGGAAAGCGGGC |
| | | AAGTGCCCCTTGCCTTGGGCTATACC |
| | | AGTATCAGCGTGGTGGGCGGGCTGCT |
| | | CGCAACCTGGGCTGGCCTGTCCCTGA |
| | | CCCGATTCTGA |
| SEQ ID | CrcB from | MIALIAAVSAGGIAGTLLRFATTNWV |
| NO: 10 | Pseudomonas | AAHWPRHFYAGTLAVNLVGCLLIGLL |
| | putida | YGLFLHKPLAPVELRAGLIVGFLGGL |
| | (PP_4001), | TTFSSFSLDTVRLMESGQVPLALGYT |
| | protein | SISVVGGLLATWAGLSLTRF* |
| | sequence | |
| SEQ ID | FIB1 from | MRESGGAAFYSADLGIIGGSGLYAFD |
| NO: 11 | Streptomyces | GLQDVTEAELRTPYGDPSGPLVLGTA |
| | sp. MA37, | GNRRIAFLPRHGRGHRIAPSQVPARA |
| | protein | NVYALKSLGVREVISVSAVGSLRGDY |
| | sequence | APGDMVVLDQLFDRTAGLRPASFFGD |
| | | GMVAHVSMADPYCGRLRPFLEQVAAE |
| | | EHPTVHGSGTYCCIEGPQFSTRAESE |
| | | VYRSWGMDVIGMTALPEAKLAREAEL |
| | | CYTGLALVTDYDCWHPGHASVDAQGV |
| | | AEIMEANVAAARRVVMRFAESSKPAP |
| | | CVCQRALDKAIMTDSQIIQGGPGNTV |
| | | PTLLRDRIAPVV* |
| SEQ ID | fIB1 from | ATGCGAGAATCGGGCGGCGCGGCCTT |
| NO: 12 | Streptomyces | CTACTCGGCCGACCTCGGAATCATCG |
| | sp. MA37, | GCGGCAGCGGGCTCTACGCCTTCGAC |
| | native | GGCCTGCAGGACGTGACGGAGGCCGA |
| | | ACTGCGGACCCCGTACGGCGACCCCA |
| | | GCGGGCCGCTGGTCCTGGGCACGGCC |
| | | GGGAACCGCCGCATCGCCTTCCTGCC |
| | | GCGCCACGGGCGCGGCCACCGCATAG |
| | | CCCCGTCGCAGGTCCCGGCGCGGGCC |
| | | AACGTCTACGCGCTGAAGTCCCTCGG |
| | | CGTCCGCGAAGTGATCAGCGTCAGCG |
| | | CCGTGGGCAGCCTGCGCGGCGACTAT |
| | | GCCCCGGGCGACATGGTCGTCCTCGA |
| | | CCAGCTCTTCGACCGCACCGCCGGGC |
| | | TGCGGCCCGCCTCGTTCTTCGGGGAC |
| | | GGGATGGTGGCCCACGTGTCGATGGC |
| | | GGACCCGTACTGCGGCCGGCTGCGCC |
| | | CCTTCCTCGAACAGGTGGCGGCCGAG |
| | | GAGCATCCCACGGTCCACGGGTCCGG |
| | | GACGTACTGCTGCATCGAGGGGCCCC |
| | | AGTTCTCCACCCGCGCCGAGTCCGAG |
| | | GTGTACCGCTCATGGGGGATGGACGT |
| | | CATCGGCATGACGGCGCTGCCGGAGG |
| | | CGAAACTCGCCCGTGAGGCTGAACTC |
| | | TGCTACACCGGGCTCGCCCTCGTGAC |
| | | GGACTACGACTGCTGGCACCCGGGCC |
| | | ACGCGTCGGTGGACGCCCAGGGGGTC |
| | | GCGGAGATCATGGAGGCCAACGTGGC |
| | | CGCCGCCCGGAGGGTCGTCATGCGGT |
| | | TCGCCGAGTCGTCGAAGCCCGCGCCC |
| | | TGCGTGTGTCAGCGGGCGCTGGACAA |
| | | AGCCATTATGACGGACAGTCAGATCA |
| | | TTCAGGGAGGTCCGGGGAACACGGTG |
| | | CCCACGCTCCTGCGCGACCGGATCGC |
| | | GCCGGTGGTGTGA |
| SEQ ID | fIB1 from | ATGCGCGAGAGCGGCGGCGCCGCCTT |
| NO: 13 | Streptomyces | CTACAGCGCCGATCTGGGCATCATCG |
| | sp. MA37, | GCGGCAGCGGCCTGTACGCCTTCGAC |
| | codon | GGCCTGCAAGACGTGACCGAAGCCGA |
| | optimized | ACTGCGCACCCCGTACGGCGACCCGA |
| | | GCGGCCCGCTGGTGCTGGGCACCGCC |
| | | GGCAACCGTCGTATCGCGTTCCTGCC |
| | | GCGCCATGGCCGCGGCCACCGTATCG |
| | | CCCCGAGCCAGGTGCCGGCCCGTGCC |
| | | AACGTGTATGCCCTGAAGAGCCTGGG |
| | | CGTGCGTGAGGTGATCAGCGTGTCCG |
| | | CCGTGGGCTCCCTGCGCGGCGACTAT |
| | | GCCCCCGGTGACATGGTGGTGCTGGA |
| | | TCAGCTGTTCGACCGCACCGCGGGCC |
| | | TGCGTCCGGCCAGCTTCTTCGGCGAT |
| | | GGCATGGTGGCCCATGTGTCGATGGC |
| | | CGACCCGTATTGCGGCCGTCTGCGCC |
| | | CGTTCCTGGAACAGGTCGCCGCCGAG |
| | | GAACACCCGACCGTGCATGGCAGCGG |
| | | CACCTACTGCTGCATCGAGGGCCCGC |
| | | AGTTCAGCACCCGCGCCGAAAGCGAG |
| | | GTGTACCGCAGCTGGGGCATGGACGT |
| | | CATCGGCATGACCGCCCTGCCCGAGG |
| | | CCAAACTGGCGCGTGAAGCCGAGCTG |
| | | TGCTATACCGGCCTGGCCCTGGTGAC |
| | | CGATTACGACTGCTGGCACCCCGGCC |
| | | ACGCCAGCGTGGATGCCCAGGGCGTG |
| | | GCCGAAATCATGGAGGCGAACGTGGC |
| | | CGCCGCCCGCCGTGTGGTGATGCGTT |
| | | TCGCGGAAAGCAGCAAGCCCGCCCCG |
| | | TGCGTCTGCCAGCGTGCCCTGGATAA |
| | | GGCCATCATGACCGATAGCCAGATCA |
| | | TCCAGGGCGGCCCCGGTAACACCGTG |
| | | CCGACCCTGCTGCGCGATCGCATCGC |
| | | CCCGGTCGTGTGA |
| SEQ ID | deoD from | ATGGCTACCCCACACATTAATGCAGA |
| NO: 14 | E. coli | AATGGGCGATTTCGCTGACGTAGTTT |
| | MG1655 | TGATGCCAGGCGACCCGCTGCGTGCG |
| | | AAGTATATTGCTGAAACTTTCCTTGA |
| | | AGATGCCCGTGAAGTGAACAACGTTC |
| | | GCGGTATGCTGGGCTTCACCGGTACT |
| | | TACAAAGGCCGCAAAATTTCCGTAAT |
| | | GGGTCACGGTATGGGTATCCCGTCCT |
| | | GCTCCATCTACACCAAAGAACTGATC |
| | | ACCGATTTCGGCGTGAAGAAAATTAT |
| | | CCGCGTGGGTTCCTGTGGCGCAGTTC |
| | | TGCCGCACGTAAAACTGCGCGACGTC |
| | | GTTATCGGTATGGGTGCCTGCACCGA |
| | | TTCCAAAGTTAACCGCATCCGTTTTA |
| | | AAGACCATGACTTTGCCGCTATCGCT |
| | | GACTTCGACATGGTGCGTAACGCAGT |
| | | AGATGCAGCTAAAGCACTGGGTATTG |
| | | ATGCTCGCGTGGGTAACCTGTTCTCC |
| | | GCTGACCTGTTCTACTCTCCGGACGG |
| | | CGAAATGTTCGACGTGATGGAAAAAT |
| | | ACGGCATTCTCGGCGTGGAAATGGAA |
| | | GCGGCTGGTATCTACGGCGTCGCTGC |
| | | AGAATTTGGCGCGAAAGCCCTGACCA |
| | | TCTGCACCGTATCTGACCACATCCGC |
| | | ACTCACGAGCAGACCACTGCCGCTGA |
| | | GCGTCAGACTACCTTCAACGACATGA |
| | | TCAAAATCGCACTGGAATCCGTTCTG |
| | | CTGGGCGATAAAGAGCATCATCACCA |
| | | CCACCATTGA |
| SEQ ID | DeoD from | MATPHINAEMGDFADVVLMPGDPLRA |
| NO: 15 | E. coli | KYIAETFLEDAREVNNVRGMLGFTGT |
| | MG1655 | YKGRKISVMGHGMGIPSCSIYTKELI |
| | | TDFGVKKIIRVGSCGAVLPHVKLRDV |
| | | VIGMGACTDSKVNRIRFKDHDFAAIA |
| | | DFDMVRNAVDAAKALGIDARVGNLFS |
| | | ADLFYSPDGEMFDVMEKYGILGVEME |
| | | AAGIYGVAAEFGAKALTICTVSDHIR |
| | | THEQTTAAERQTTFNDMIKIALESVL |
| | | LGDKE* |
| SEQ ID | pfs from | ATGAAAATCGGCATCATTGGTGCAAT |
| NO: 16 | E. coli | GGAAGAAGAAGTTACGCTGCTGCGTG |
| | MG1655 | ACAAAATCGAAAACCGTCAAACTATC |
| | | AGTCTCGGCGGTTGCGAAATCTATAC |
| | | CGGCCAACTGAATGGAACCGAGGTTG |
| | | CGCTTCTGAAATCGGGCATCGGTAAA |
| | | GTCGCTGCGGCGCTGGGTGCCACTTT |
| | | GCTGTTGGAACACTGCAAGCCAGATG |
| | | TGATTATTAACACCGGTTCTGCCGGT |
| | | GGCCTGGCACCAACGTTGAAAGTGGG |
| | | CGATATCGTTGTCTCGGACGAAGCAC |
| | | GTTATCACGACGCGGATGTCACGGCA |
| | | TTTGGTTATGAATACGGTCAGTTACC |
| | | AGGCTGTCCGGCAGGCTTTAAAGCTG |
| | | ACGATAAACTGATCGCTGCCGCTGAG |
| | | GCCTGCATTGCCGAACTGAATCTTAA |
| | | CGCTGTACGTGGCCTGATTGTTAGCG |
| | | GCGACGCTTTCATCAACGGTTCTGTT |
| | | GGTCTGGCGAAAATCCGCCACAACTT |
| | | CCCACAGGCCATTGCTGTAGAGATGG |
| | | AAGCGACGGCAATCGCCCATGTCTGC |
| | | CACAATTTCAACGTCCCGTTTGTTGT |
| | | CGTACGCGCCATCTCCGACGTGGCCG |
| | | ATCAACAGTCTCATCTTAGCTTCGAT |
| | | GAGTTCCTGGCTGTTGCCGCTAAACA |
| | | GTCCAGCCTGATGGTTGAGTCACTGG |
| | | TGCAGAAACTTGCACATGGCCATCAT |
| | | CACCACCACCATTGA |
| SEQ ID | Pfs from | MKIGIIGAMEEEVTLLRDKIENRQTI |
| NO: 17 | E. coli | SLGGCEIYTGQLNGTEVALLKSGIGK |
| | MG1655 | VAAALGATLLLEHCKPDVIINTGSAG |
| | | GLAPTLKVGDIVVSDEARYHDADVTA |
| | | FGYEYGQLPGCPAGFKADDKLIAAAE |
| | | ACIAELNLNAVRGLIVSGDAFINGSV |
| | | GLAKIRHNFPQAIAVEMEATAIAHVC |
| | | HNFNVPFVVVRAISDVADQQSHLSFD |
| | | EFLAVAAKQSSLMVESLVQKLAHG* |

### Items

1. A cell capable of producing a fluorinated, a chlorinated or a brominated compound from a substrate in the presence of fluoride, chloride or bromide, respectively, said cell comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
b) a second nucleic acid comprising a riboswitch, wherein transcription of the fluorinase gene from the first promoter is induced in the presence of an inducer, wherein said riboswitch is responsive to said inducer;
wherein the cell is capable of expressing the activator of transcription and the fluorinase at least in the presence of said inducer.

2. The cell according to item 1, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

3. The cell according to any one of the preceding items, wherein the first nucleic acid and the second nucleic acid are the same nucleic acid.

4. The cell according to any one of the preceding items, wherein the first nucleic acid further comprises a phosphorylase gene such as flB1 or deoD, said gene encoding a phosphorylase (EC 2.4.2.28) capable of catalysing the phosphorylation of a fluorinated or a chlorinated substrate such as a fluorinated or chlorinated adenosine, said phosphorylase gene being under control of the first promoter.

5. The cell according to any one of the preceding items, wherein the first nucleic acid further comprises a gene encoding a nucleosidase (EC 3.2.2.9) such as Pfs, said nucleosidase being capable of catalysing the conversion of 5′-fluoro-5′-deoxyadenosine to 5′-fluorodeoxyribose, said nucleosidase gene being under control of the first promoter.

6. The cell according to any one of the preceding items, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

7. A cell capable of producing a fluorinated and phosphorylated compound, a chlorinated and phosphorylated compound, or a brominated and phosphorylated compound from a substrate, said cell comprising:
a) a first nucleic acid comprising a first promoter and a phosphorylase gene such as flB1 or deoD, said gene encoding a phosphorylase (EC 2.4.2.28) capable of catalysing the phosphorylation of a fluorinated, chlorinated and/or brominated adenosine, said phosphorylase gene being under control of the first promoter;
b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
wherein the cell is capable of expressing the phosphorylase at least in the presence of said inducer.

8. A cell capable of converting 5′-fluoro-5′-deoxyadenosine to 5′-fluorodeoxyribose in the presence of fluoride, said cell comprising:
a) a first nucleic acid comprising a first promoter and a gene encoding a nucleosidase (EC 3.2.2.9) such as Pfs, said nucleosidase being capable of catalysing the conversion of 5′-fluoro-5′-deoxyadenosine to 5′-fluorodeoxyribose, said nucleosidase gene being under control of the first promoter, wherein the first promoter preferably is a T7 promoter;
b) a second nucleic acid comprising a fluoride-responsive riboswitch such as the FRS riboswitch from P. syringae (SEQ ID NO: 4), a second promoter such as the native promoter of the eriC^{F} gene of P. syringae or the PEM7 promoter, and a gene encoding an activator of transcription, said gene being under the control of the second promoter,
wherein transcription of the activator of transcription can be induced by an inducer, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
wherein the cell is capable of expressing the activator of transcription and the nucleosidase gene at least in the presence of fluoride.

9. The cell according to any one of the preceding items, wherein the cell can tolerate toxic compounds, such as fluorinated compounds.

10. The cell according to any one of the preceding items, wherein the cell is a non-pathogenic organism.

11. The cell according to any one of the preceding items, wherein the cell is a mammalian cell, a plant cell, an insect cell, a yeast cell or a bacterial cell.

12. The cell according to any one of the preceding items, wherein the cell is a bacterial cell of the Pseudomonas genus, the Bacillus genus, the Vibrio genus or the Escherichia genus or a yeast cell of the genus Saccharomyces, Pichia, Yarrowia, Kluyveromyces, Candida, Rhodotorula, Rhodosporidium, Cryptococcus, Trichosporon and Lipomyces.

13. The cell according to any one of the preceding items, wherein the cell is a bacterial cell selected from Pseudomonas putida, Pseudomonas fluorescens, Pseudomonas taiwanesis Pseudomonas syringae, Pseudomonas stutzeri, Pseudomonas oleovorans, Pseudomonas mendocina, Bacillus subtilis, Bacillus cereus, Bacillus megaterium, Vibrio natriegens and Escherichia coli, or a yeast cell selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica.

14. The cell according to any one of the preceding items, wherein the cell is Pseudomonas putida KT2440.

15. The cell according to any one of items 1 to 14, wherein the fluorinase gene encodes a fluorinase derived from a Streptomyces such as Streptomyces sp. MA37, Streptomyces cattleya, Streptomyces xinghaiensis, Nocardia brasiliensis or Actinoplanes sp. N902-109.

16. The cell according to any one of the preceding items, wherein the fluorinase is capable of catalysing fluorination, chlorination or bromination of a substrate to obtain a fluorinated, a chlorinated or a brominated compound, such as 5′-deoxy-5′-fluoroadenosine, 5′-chloro-5′-deoxyadenosine, 5′-bromo-5′-deoxyadenosine, 5′-deoxy-5′-fluoro-D-ribose 1-phosphate, 5′-chloro-5′-deoxy-D-ribose 1-phosphate, or 5′-bromo-5′-deoxy-D-ribose 1-phosphate.

17. The cell according to any one of the preceding items, wherein the substrate is selected from the group consisting of S-adenosyl-L-methionine (SAM) or a derivative thereof, such as a methylaza derivative, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, ¹⁸F, chloride, 5′-deoxy-5′-fluoroadenosine, 5′-chloro-5′-deoxyadenosine, and 5′-bromo-5′-deoxyadenosine.

18. The cell according to any one of the preceding items, wherein the substrate is a SAM derivative of formula (I):

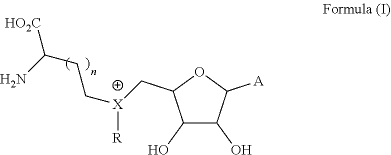

wherein:
n is 0, 1 or 2;
X is selected from the group consisting of: S, Se and NMe;
R is selected from the group consisting of: Me and propargyl;
A is a heterocycle.

19. The cell according to item 18, wherein the SAM derivative of formula (I) is of formula (II):

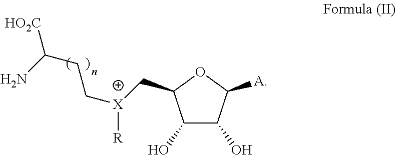

20. The cell according to any of the preceding items wherein the substrate is selected from the group consisting of

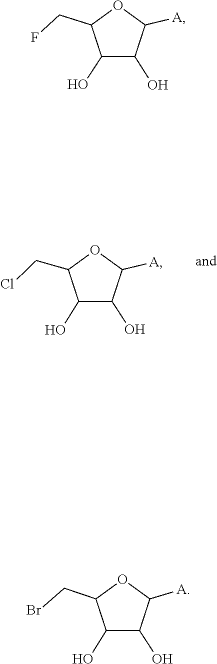

21. The cell according to any one of items 18 to 20, wherein A is selected from the group consisting of

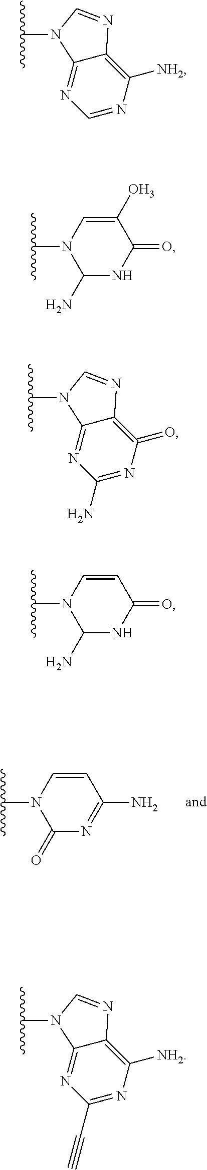

22. The cell according to any one of items 18 to 21, wherein the SAM derivative of formula (I) is of formula (III):

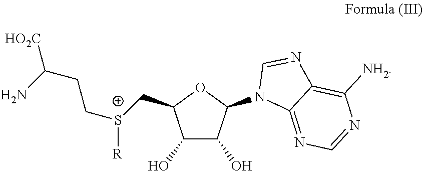

23. The cell according to any one of items 18 to 22, wherein the substrate is selected from the group consisting of

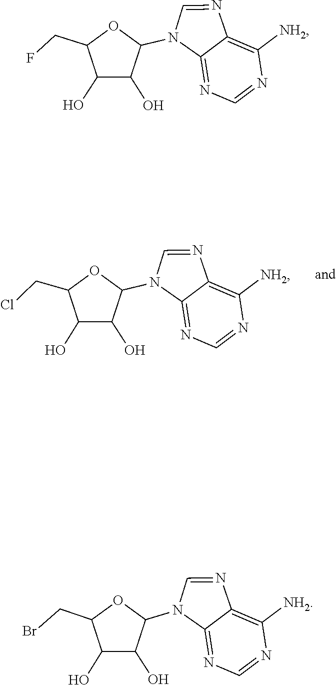 [SMILES: Nc1ncnc2c1ncn2C1OC(CF)C(O)C1O ; Nc1ncnc2c1ncn2C1OC(CCl)C(O)C1O ; Nc1ncnc2c1ncn2C1OC(CBr)C(O)C1O]

24. The cell according to any one of the preceding items, wherein the fluorinase is the fluorinase as set forth in SEQ ID NO: 1, or a functional variant thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 1.

25. The cell according to any one of the preceding items, wherein the fluorinase gene is codon-optimised.

26. The cell according to any one of the preceding items, wherein the fluorinase gene is as set forth in SEQ ID NO: 2 or SEQ ID NO: 3, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 2 or SEQ ID NO: 3.

27. The cell according to any one of the preceding items, wherein the phosphorylase is the phosphorylase as set forth in SEQ ID NO: 11 or SEQ ID NO: 15, or a functional variant thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 11 or SEQ ID NO: 15.

28. The cell according to any one of the preceding items, wherein the nucleosidase is the nucleosidase as set forth in SEQ ID NO: 17, or a functional variant thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 17.

29. The cell according to any one of the preceding items, wherein the phosphorylase gene is as set forth in SEQ ID NO: 12, SEQ ID NO: 13 or SEQ ID NO: 14, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 12, SEQ ID NO: 13 or SEQ ID NO: 14.

30. The cell according to any one of the preceding items, wherein the nucleosidase gene is as set forth in SEQ ID NO: 16, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 16.

31. The cell according to any one of the preceding items, wherein the phosphorylase gene, the nucleosidase gene and/or the fluorinase gene is codon-optimised.

32. The cell according to any one of the preceding items, wherein the activator of transcription is a polymerase such as a T7 RNA polymerase and the first promoter is the native promoter of said polymerase such as a T7 promoter.

33. The cell according to any one of the preceding items, wherein the polymerase is a T7 RNA polymerase and the first promoter is a T7 promoter.

34. The cell according to any one of the preceding items, wherein the riboswitch is the fluoride-responsive riboswitch (FRS) from Pseudomonas syringae as set forth in SEQ ID NO: 4, or a functional variant thereof having at least 90% homology to SEQ ID NO: 4, such at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 4.

35. The cell according to any one of the preceding items, wherein the second nucleic acid is as set forth in SEQ ID NO: 5, SEQ ID NO: 6, SEQ ID NO: 7 or SEQ ID NO: 8, preferably as set forth in SEQ ID NO: 5, SEQ ID NO: 6 or SEQ ID NO: 8, most preferably as set forth in SEQ ID NO: 5 or SEQ ID NO: 6; or wherein the nucleic acid comprises or consists of a sequence having at least 90% homology to SEQ ID NO: 5, SEQ ID NO: 6, SEQ ID NO: 7 or SEQ ID NO: 8, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 5, SEQ ID NO: 6, SEQ ID NO: 7 or SEQ ID NO: 8, preferably to SEQ ID NO: 5, SEQ ID NO: 6 or SEQ ID NO: 8, most preferably to SEQ ID NO: 5 or SEQ ID NO: 6.

36. The cell according to any one of the preceding items, wherein the inducer is a fluoride salt such as NaF or KF, or a chloride salt such as NaCl or KCl.

37. The cell according to any one of the preceding items, further comprising a mutation in at least one fluoride, chloride or bromide transporter gene encoding a fluoride, chloride or bromide transporter, respectively, said mutation resulting in a partial or total loss of function of said fluoride, chloride or bromide transporter, respectively.

38. The cell according to item 37, wherein the mutation is a deletion.

39. The cell according to any one of items 37 to 38, wherein the cell is a Pseudomonas putida cell and the fluoride transporter gene is the crcB gene as set forth in SEQ ID NO: 9.

40. An expression system for expression in a cell, comprising:
a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
b) a second nucleic acid comprising a riboswitch, wherein transcription of the fluorinase gene from the first promoter is induced in the presence of an inducer, wherein said riboswitch is responsive to said inducer.

41. The expression system in a cell according to item 40, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

42. The expression system in a cell according to any one of items 40 to 41, wherein the first nucleic acid and the second nucleic acid are the same nucleic acid.

43. The expression system in a cell according to any one of items 40 to 42, wherein the first nucleic acid further comprises a phosphorylase gene such as flB1 or deoD, said gene encoding a phosphorylase (EC 2.4.2.28) capable of catalysing the phosphorylation of a fluorinated or a chlorinated adenosine, said phosphorylase gene being under control of the first promoter.

44. The expression system in a cell according to any one of items 40 to 43, wherein the first nucleic acid further comprises a gene encoding a nucleosidase (EC 3.2.2.9) such as Pfs, said nucleosidase being capable of catalysing the conversion of 5′-fluoro-5′-deoxyadenosine to 5′-fluorodeoxyribose, said nucleosidase gene being under control of the first promoter.

45. The expression system according to any one of items 40 to 44, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

46. An expression system for expression in a cell, comprising:
a. a first nucleic acid comprising a first promoter and a nucleosidase gene such as Pfs, said gene encoding a nucleosidase (EC 3.2.2.9) capable of catalysing the conversion of 5′-fluoro-5′-deoxyadenosine to 5′-fluorodeoxyribose, said nucleosidase gene being under control of the first promoter;
b. a second nucleic acid comprising a riboswitch, wherein the riboswitch is fluoride-responsive and is capable of inducing transcription of the nucleosidase gene at least in the presence of fluoride.

47. An expression system for expression in a cell, comprising:
a. a first nucleic acid comprising a first promoter and a phosphorylase gene such as flB1 or deoD, said gene encoding a phosphorylase (EC 2.4.2.28) capable of catalysing the phosphorylation of a fluorinated, chlorinated and/or brominated adenosine, said phosphorylase gene being under control of the first promoter;
b. a second nucleic acid comprising a riboswitch, wherein the riboswitch is fluoride-responsive and is capable of inducing transcription of the phosphorylase gene at least in the presence of fluoride.

48. The expression system according to any one of items 46 to 47, wherein the second nucleic acid further comprises a second promoter and a gene encoding an activator of transcription, said gene being under the control of the second promoter, wherein transcription of the activator of transcription can be induced by an inducer, wherein the activator of transcription upon expression activates transcription from the first promoter.

49. The expression system of items 46 to 48, wherein the cell is as defined in any one of items 1 to 39.

50. The expression system of any one of items 46 to 49, wherein the first nucleic acid and/or the second nucleic acid are independently comprised in a vector or are integrated in the genome of the cell.

51. A method for in vivo fluorination, chlorination or bromination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, a C—Cl bond and/or a C—Br bond, said fluorinase gene being under the control of the first promoter;
   b) a second nucleic acid comprising a riboswitch, wherein transcription of the fluorinase gene from the first promoter is induced in the presence of an inducer, wherein said riboswitch is responsive to said inducer;
ii) adding the inducer to the medium and incubating the cell in the presence of inducer and a co-substrate selected from a fluoride salt, a chloride salt or a bromide salt,
   whereby transcription of the fluorinase gene is induced,
   thereby inducing fluorination, chlorination or bromination of the substrate to yield a fluorinated, a chlorinated or a brominated product.

52. A method for in vivo production of 5′-fluorodeoxyribose, 5′-chlorodeoxyribose and/or 5′-bromodeoxyribose, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, a C—Cl bond and/or a C—Br bond, and a gene encoding a nucleosidase (EC 3.2.2.9) such as Pfs, said nucleosidase being capable of catalysing the conversion of 5′-fluoro-5′-deoxyadenosine to 5′-fluorodeoxyribose, and/or conversion of 5′-chloro-5′-deoxyadenosine to 5′-chlorodeoxyribose and/or conversion of 5′-bromo-5′-deoxyadenosine to 5′-bromodeoxyribose, said fluorinase gene and said nucleosidase gene being under the control of the first promoter;
   b) a second nucleic acid comprising a riboswitch, wherein transcription of the fluorinase gene from the first promoter is induced in the presence of an inducer, wherein said riboswitch is responsive to said inducer;
ii) adding the inducer to the medium and incubating the cell in the presence of inducer and a co-substrate selected from a fluoride salt, a chloride salt or a bromide salt,
   whereby transcription of the fluorinase gene and of the nucleosidase is induced,
   thereby inducing production of 5′-fluoro-5′-deoxyadenosine and its conversion to 5′-fluorodeoxyribose, and/or production of 5′-chloro-5′-deoxyadenosine and its conversion to 5′-chlorodeoxyribose and/or production of 5′-bromo-5′-deoxyadenosine and its conversion to 5′-bromodeoxyribose.

53. The method according to any one of items 51 to 52, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene.

54. The method according to any one of items 51 to 53, wherein the first nucleic acid and the second nucleic acid are the same nucleic acid.

55. The method according to any one of items 51 to 54, wherein the first nucleic acid further comprises a phosphorylase gene such as flB1 or deoD, said gene encoding a phosphorylase (EC 2.4.2.28) capable of catalysing the phosphorylation of a fluorinated or a chlorinated substrate such as a fluorinated or chlorinated adenosine, said gene being under control of the first promoter.

56. The method according to any one of items 51 to 55, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene, the nucleosidase gene and/or the phosphorylase gene.

57. A method for in vivo phosphorylation of a fluorinated, a chlorinated, or brominated substrate, comprising the steps of:
i) propagating a cell in a medium, said cell comprising:
   a) a first nucleic acid comprising a first promoter and a phosphorylase gene such as flB1 or deoD, said gene encoding a phosphorylase (EC 2.4.2.28) capable of catalysing the phosphorylation of a fluorinated, chlorinated and/or brominated substrate such as a fluorinated, chlorinated and/or brominated adenosine, said gene being under control of the first promoter;
   b) a second nucleic acid comprising a riboswitch;
wherein the cell is capable of expressing the phosphorylase at least in the presence of said inducer.
ii) adding the inducer to the medium and incubating the cell in the presence of inducer,
   whereby transcription of the phosphorylase gene is induced,
   thereby inducing phosphorylation of the substrate to yield a fluorinated and phosphorylated product, a chlorinated and phosphorylated product, or a brominated and phosphorylated product.

58. The method according to item 57, wherein the second nucleic acid further comprises a second promoter and a gene encoding an activator of transcription, said gene being under the control of the second promoter, wherein transcription of the activator of transcription can be induced by an inducer, wherein the activator of transcription upon expression activates transcription from the first promoter.

59. The method according to items 51 to 58, wherein the cell is as defined in any one of items 1 to 39, or wherein the cell comprises an expression system as defined in any one of items 40 to 50.

60. The method according to any one of items 51 to 59, wherein the inducer is the co-substrate.

61. The method according to any one of items 51 to 60, wherein the method is for in vivo fluorination of the substrate, and the co-substrate or inducer is a fluoride salt such as NaF or KF.

62. The method according to any one of items 51 to 61, wherein NaF or KF is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

63. The method according to any one of items 51 to 62, wherein the method is for in vivo chlorination of the substrate, and the co-substrate or inducer is a chloride salt such as NaCl or KCl.

64. The method according to any one of items 51 to 63, wherein NaCl or KCl is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

65. The method according to any one of items 51 to 64, wherein the method is for in vivo bromination of the substrate, and the co-substrate or inducer is a bromide salt such as NaBr or KBr.

66. The method according to any one of items 51 to 65, wherein NaBr or KBr is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

67. The method according to any one of items 51 to 66, wherein the inducer and/or co-substrate is added at the culture onset or in mid-exponential phase.

68. The method according to any one of items 51 to 67, wherein the cell is incubated in the presence of inducer and/or co-substrate for at least 4 hours, such as at least 8 hours, such as at least 12 hours, such as at least 16 hours, such as at least 20 hours, such as at least 24 hours, such as at least 36 hours, such as at least 48 hours, or more.

69. The method according to any one of items 51 to 68, further comprising a step of recovering the fluorinated, chlorinated or brominated product.

70. A fluorinated, a chlorinated or a brominated product, or 5′-fluorodeoxyribose, 5′-chlorodeoxyribose or 5′-bromodeoxyribose obtainable by the method according to any one of items 51 to 69.

71. A composition comprising a fluorinated, a chlorinated or a brominated product according to item 70.

72. A method for manufacturing a fluorinated, a chlorinated or a brominated compound of interest, said method comprising the steps of:
i) providing a fluorinated, a chlorinated or a brominated product by the method of any one of items 51 to 69; and
i) optionally converting said fluorinated, chlorinated or brominated product to the fluorinated, chlorinated or brominated compound of interest.

73. The fluorinated, chlorinated or brominated product or the 5′-fluorodeoxyribose, 5′-chlorodeoxyribose or 5′-bromodeoxyribose according to item 70 or the composition according to item 71 for use as a medicament.

1. A cell capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch,
wherein transcription of the fluorinase gene from the first promoter is induced in the presence of an inducer, wherein said riboswitch is responsive to said inducer;
wherein the cell is capable of expressing the fluorinase at least in the presence of said inducer.

2. The cell according to claim 1, wherein the first nucleic acid and the second nucleic acid are the same nucleic acid or wherein the first nucleic acid and the second nucleic acid are different nucleic acids.

3. The cell according to any one of the preceding claims, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

4. The cell according to any one of the preceding claims, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

5. The cell according to any one of the preceding claims, wherein the cell can tolerate toxic compounds, such as fluorinated compounds.

6. The cell according to any one of the preceding claims, wherein the cell is a non-pathogenic organism.

7. The cell according to any one of the preceding claims, wherein the cell is a mammalian cell, a plant cell, an insect cell, a yeast cell or a bacterial cell.

8. The cell according to any one of the preceding claims, wherein the cell is a bacterial cell of the Pseudomonas genus, the Bacillus genus, the Vibrio genus or the Escherichia genus or a yeast cell of the genus Saccharomyces, Pichia, Yarrowia, Kluyveromyces, Candida, Rhodotorula, Rhodosporidium, Cryptococcus, Trichosporon or Lipomyces.

9. The cell according to any one of the preceding claims, wherein the cell is a bacterial cell selected from Pseudomonas putida, Pseudomonas fluorescens, Pseudomonas taiwanesis Pseudomonas syringae, Pseudomonas stutzeri, Pseudomonas oleovorans, Pseudomonas mendocina, Bacillus subtilis, Bacillus cereus, Bacillus megaterium, Vibrio natriegens and Escherichia coli, or a yeast cell selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica, preferably the cell is Pseudomonas putida KT2440.

10. The cell according to any one of the preceding claims, wherein the cell is Pseudomonas putida KT2440.

11. The cell according to any one of the preceding claims, wherein the fluorinase gene encodes a fluorinase derived from a Streptomyces such as Streptomyces sp. MA37, Streptomyces cattleya, Streptomyces xinghaiensis, Nocardia brasiliensis or Actinoplanes sp. N902-109.

12. The cell according to any one of the preceding claims, wherein the fluorinase is capable of catalysing fluorination of a substrate such as adenosine to obtain a fluorinated compound, such as 5′-deoxy-5′-fluoroadenosine.

13. The cell according to any one of the preceding claims, wherein the substrate is selected from the group consisting of S-adenosyl-L-methionine (SAM) or a derivative thereof, such as a methylaza derivative, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, ¹⁸F, and chloride.

14. The cell according to any one of the preceding claims, wherein the substrate is a SAM derivative of formula (I): 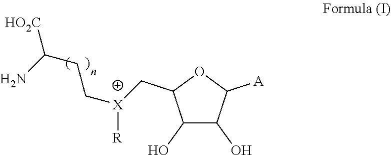
wherein:
n is 0, 1 or 2;
X is selected from the group consisting of: S, Se and NMe;
R is selected from the group consisting of: Me and propargyl;
A is a heterocycle.

15. The cell according to claim 14, wherein the SAM derivative of formula (I) is of formula (II):

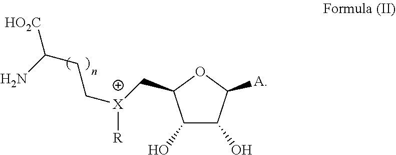

16. The cell according to any one of claims 14 to 15, wherein A is selected from the group consisting of

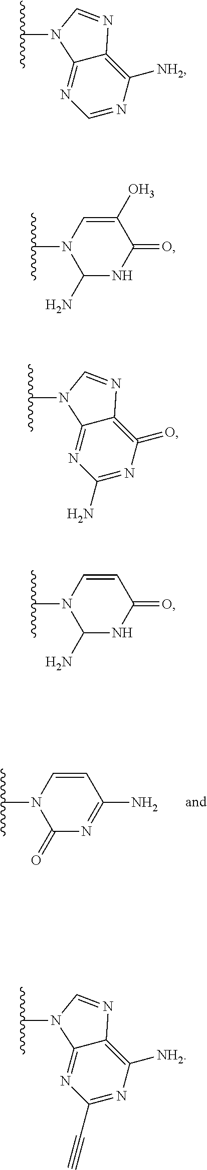

17. The cell according to any one of claims 14 to 16, wherein the SAM derivative of formula (I) is of formula (III):

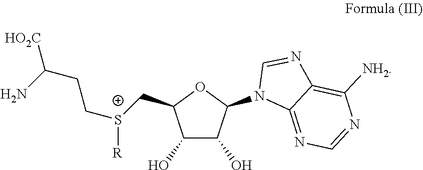

18. The cell according to any one of the preceding claims, wherein the fluorinase is the fluorinase as set forth in SEQ ID NO: 1, or a functional variant thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 1.

19. The cell according to any one of the preceding claims, wherein the fluorinase gene is codon-optimised.

20. The cell according to any one of the preceding claims, wherein the fluorinase gene is as set forth in SEQ ID NO: 2 or SEQ ID NO: 3, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 2 or SEQ ID NO: 3.

21. The cell according to any one of the preceding claims, wherein the first nucleic acid further comprises a phosphorylase gene such as flB1 or deoD, said gene encoding a phosphorylase (EC 2.4.2.28) capable of catalysing the phosphorylation of a fluorinated substrate such as a fluorinated adenosine, said gene being under control of the first promoter, whereby the cell is capable of producing a phosphorylated and fluorinated compound such as 5′-deoxy-5′-fluoro-D-ribose 1-phosphate

22. The cell according to any one of the preceding claims, wherein the phosphorylase gene is codon optimised.

23. The cell according to any one of the preceding claims, wherein the gene encoding the phosphorylase is the flB1 gene as set forth in SEQ ID NO: 12 or SEQ ID NO: 13, or the deoD gene as set forth in SEQ ID NO: 14, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 12, SEQ ID NO:
13 or SEQ ID NO: 14.

24. The cell according to any one of the preceding claims, wherein the first nucleic acid further comprises a gene encoding a nucleosidase (EC 3.2.2.9) such as Pfs, said nucleosidase being capable of catalysing the conversion of 5′-fluoro-5′-deoxyadenosine to 5′-fluorodeoxyribose, said nucleosidase gene being under control of the first promoter, optionally wherein the gene encoding the nucleosidase is codon-optimised.

25. The cell according to claim 24, wherein the nucleosidase gene is the pfs gene as set forth in SEQ ID NO: 16, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 16.

26. The cell according to any one of the preceding claims, wherein the activator of transcription is a polymerase such as a T7 RNA polymerase and the first promoter is the native promoter of said polymerase such as a T7 promoter.

27. The cell according to any one of the preceding claims, wherein the polymerase is a T7 RNA polymerase and the first promoter is a T7 promoter.

28. The cell according to any one of the preceding claims, wherein the riboswitch is the fluoride-responsive riboswitch (FRS) from Pseudomonas syringae as set forth in SEQ ID NO: 4, or a functional variant thereof having at least 90% homology to SEQ ID NO: 4, such at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 4.

29. The cell according to any one of the preceding claims, wherein the second nucleic acid is as set forth in SEQ ID NO: 5, SEQ ID NO: 6, SEQ ID NO: 7 or SEQ ID NO: 8, preferably as set forth in SEQ ID NO: 5, SEQ ID NO: 6 or SEQ ID NO: 8, most preferably as set forth in SEQ ID NO: 5 or SEQ ID NO: 6; or wherein the nucleic acid comprises or consists of a sequence having at least 90% homology to SEQ ID NO: 5, SEQ ID NO: 6, SEQ ID NO: 7 or SEQ ID NO: 8, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 5, SEQ ID NO: 6, SEQ ID NO: 7 or SEQ ID NO: 8, preferably to SEQ ID NO: 5, SEQ ID NO: 6 or SEQ ID NO: 8, most preferably to SEQ ID NO: 5 or SEQ ID NO: 6.

30. The cell according to any one of the preceding claims, wherein the inducer is a fluoride salt such as NaF or KF.

31. The cell according to any one of the preceding claims, further comprising a mutation in at least one fluoride transporter gene encoding a fluoride transporter, said mutation resulting in a partial or total loss of function of said fluoride transporter.

32. The cell according to claim 31, wherein the mutation is a deletion.

33. The cell according to any one of claims 31 to 32, wherein the cell is a Pseudomonas putida cell and the fluoride transporter gene is the crcB gene as set forth in SEQ ID NO: 9.

34. An expression system for expression in a cell, comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch,
wherein transcription of the fluorinase gene from the first promoter is induced in the presence of an inducer, wherein said riboswitch is responsive to said inducer, whereby the cell is capable of producing a fluorinated product such as 5′-fluoro-5′-deoxyadenosine or 5′-deoxy-5′-fluoro-D-ribose 1-phosphate.

35. The expression system according to claim 34, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

36. The expression system according to any one of claims 34 to 35, wherein the first nucleic acid and the second nucleic acid are the same nucleic acid.

37. The expression system according to any one of claims 34 to 36, wherein the first nucleic acid further comprises a gene encoding a phosphorylase such as flB1 or deoD, said phosphorylase (EC 2.4.2.28) being capable of catalysing the phosphorylation of a fluorinated or a chlorinated adenosine and/or a gene encoding a nucleosidase (EC 3.2.2.9) such as Pfs, said nucleosidase being capable of catalysing the conversion of 5′-fluoro-5′-deoxyadenosine to 5′-fluorodeoxyribose, said phosphorylase gene and/or said nucleosidase gene being under control of the first promoter.

38. The expression system in a cell according to any one of claims 34 to 37, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.

39. The expression system according to any one of claims 34 to 38, wherein the gene encoding the phosphorylase is the flB1 gene as set forth in SEQ ID NO: 12 or SEQ ID NO: 13, or the deoD gene as set forth in SEQ ID NO: 14, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 12, SEQ ID NO: 13 or SEQ ID NO: 14.

40. The expression system according to any one of claims 34 to 39, wherein the nucleosidase gene is as set forth in SEQ ID NO: 16, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 16.

41. The expression system of any one of claims 34 to 40, wherein the cell is as defined in any one of claims 1 to 33.

42. The expression system of any one of claims 34 to 41, wherein the first nucleic acid and/or the second nucleic acid are independently comprised in a vector or are integrated in the genome of the cell.

43. A method for in vivo fluorination of a substrate, comprising the steps of:
i) propagating a cell in a medium, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) a second nucleic acid comprising a riboswitch,
wherein transcription of the fluorinase gene from the first promoter is induced in the presence of an inducer, wherein said riboswitch is responsive to said inducer;
ii) adding the inducer to the medium and incubating the cell in the presence of inducer and a co-substrate selected from a fluoride salt, wherein optionally the inducer is the co-substrate,
whereby transcription of the fluorinase gene is induced,
thereby inducing fluorination of the substrate to yield a fluorinated product.

44. The method according to claim 43, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene.

45. The method according to any one of claims 43 to 44, wherein the first nucleic acid and the second nucleic acid are the same nucleic acid.

46. The method according to any one of claims 43 to 45, wherein the first nucleic acid further comprises a gene encoding a phosphorylase such as FlB1 or DeoD, said phosphorylase (EC 2.4.2.28) being capable of catalysing the phosphorylation of a fluorinated substrate such as a fluorinated adenosine, said gene being under control of the first promoter.

47. The method according to any one of claims 43 to 46, wherein the first nucleic acid further comprises a gene encoding a nucleosidase (EC 3.2.2.9) such as Pfs, said nucleosidase being capable of catalysing the conversion of 5′-fluoro-5′-deoxyadenosine to 5′-fluorodeoxyribose, said nucleosidase gene being under control of the first promoter.

48. The method for in vivo fluorination according to any one of claims 43 to 47, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene and the phosphorylase gene.

49. The method according to any one of claims 43 to 48, wherein the gene encoding the phosphorylase is the flB1 gene as set forth in SEQ ID NO: 12 or SEQ ID NO: 13, or the deoD gene as set forth in SEQ ID NO: 14, or a homologue thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 12, SEQ ID NO: 13 or SEQ ID NO: 14.

50. The method according to any one of claims 43 to 49, wherein the nucleosidase is the nucleosidase as set forth in SEQ ID NO: 17, or a functional variant thereof having at least 80% homology thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology to SEQ ID NO: 17.

51. The method according to any one of claims 43 to 50, wherein the cell is as defined in any one of claims 1 to 33, or wherein the cell comprises an expression system as defined in claims 34 to 42.

52. The method according to any one of claims 34 to 51, wherein the inducer is the co-substrate.

53. The method according to any one of claims 34 to 52, wherein the method is for in vivo fluorination of the substrate, and the co-substrate or inducer is a fluoride salt such as NaF or KF.

54. The method according to any one of claims 34 to 53, wherein the inducer and/or co-substrate is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

55. The method according to any one of claims 34 to 54, wherein NaF or KF is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

56. The method according to any one of claims 34 to 55, wherein the inducer and/or co-substrate is added at the culture onset or in mid-exponential phase.

57. The method according to any one of claims 34 to 56, wherein the cell is incubated in the presence of inducer and/or co-substrate for at least 4 hours, such as at least 8 hours, such as at least 12 hours, such as at least 16 hours, such as at least 20 hours, such as at least 24 hours, such as at least 36 hours, such as at least 48 hours, or more.

58. The method according to any one of claims 34 to 57, further comprising a step of recovering the product.

59. A fluorinated product obtainable by the method according to any one of claims 34 to 58.

60. A composition comprising a fluorinated product according to claim 59.

61. A method for manufacturing a fluorinated compound of interest, said method comprising the steps of:
i) providing a fluorinated product by the method of any one of claims 34 to 58; and
ii) optionally converting said fluorinated product to the fluorinated compound of interest.

62. The fluorinated product according to claim 59 or the composition according to claim 60 for use as a medicament.
